SEQUENCE LISTING (2 sequences)

SEQ ID NO: 1 - protein, 106 aa, Homo sapiens
RLINQPLPDLKNVKSKIGSTDNIKYQPKGGQVQIVTKKIDLSHVTSKCGSLKNIRHRPGGGRVKIESVKLDFKEKAQAKVGSLDNAHHVPGGGNVKIDSQKLNFRE

SEQ ID NO: 2 - protein, 136 aa, Homo sapiens
AAATTRKPESNAVTKTAGPIASAQKQPAGKVQIVSKKVSYSHIQSKCGSKDNIKHVPGGGNVQIQNKKVDISKVSSKCGSKANIKHKPGGGDVKIESQKLNFKEKAQAKVGSLDNVGHLPAGGAVKTEGGGSEAPL

(19) United States
(12) Patent Application Publication
(10) Pub. No.: US 2026/0308210 A1
(43) Pub. Date: Oct. 8, 2026

(54) HOMOLOGY-BASED CAPPERS FOR BLOCKING TAU AGGREGATION AND IDENTIFYING DISEASE-SPECIFIC TAU FILAMENTS

(71) Applicant: University of Denver, Denver, CO (US)

(72) Inventors: Martin Margittai, Englewood, CO (US); Jasdeep Singh, Denver, CO (US); Zachariah Yasir Gabani, Littleton, CO (US)

(21) Appl. No.: 19/562,584

(22) Filed: Mar. 10, 2026

Related U.S. Application Data
(60) Provisional application No. 63/769,860, filed on Mar. 11, 2025.

(51) Int. Cl. C07K 14/47; A61K 38/00; G01N 33/68

(52) U.S. Cl. CPC C07K 14/47; G01N 33/6854; A61K 38/00

(57) ABSTRACT

A polypeptide capable of inhibiting Tau aggregation includes an amino acid sequence sharing between 20% and 99% sequence similarity with a structured core region of a pathological Tau filament, where the polypeptide is capable of binding to an end of a Tau fibril and reducing Tau aggregation or Tau seeding activity. The polypeptide may include an amino acid sequence derived from a microtubule-associated protein such as MAP2 or MAP4. Methods for inhibiting Tau aggregation in a biological sample include contacting the biological sample containing Tau with the polypeptide, where the polypeptide binds to an end of a Tau fibril and reduces Tau aggregation relative to a control sample.

### CROSS-REFERENCE TO RELATED APPLICATIONS

This application claims priority to U.S. Provisional Application No. 63/769,860, titled Homology-based cappers for blocking Tau aggregation and identifying disease-specific Tau filaments, filed Mar. 11, 2025, which is hereby incorporated by reference in its entirety.

### INCORPORATION BY REFERENCE OF SEQUENCE LISTING

The Sequence Listing XML file named “2024-012-01US Sequence Listing XML v1.xml”, created on Jun. 15, 2026, and having a size of 3,055 bytes, is hereby incorporated by reference in its entirety.

### FIELD OF THE DISCLOSURE

The present disclosure relates generally to therapeutic and diagnostic compositions for neurodegenerative diseases. In particular, but not by way of limitation, the present disclosure relates to homology-based protein molecules derived from microtubule-associated proteins MAP2 and MAP4 for inhibiting Tau fibril aggregation in tauopathies including Alzheimer's disease, progressive supranuclear palsy, and Pick's disease, and methods and systems associated therewith.

### DESCRIPTION OF RELATED ART

Tauopathies include a group of neurodegenerative disorders characterized by abnormal aggregation and deposition of Tau protein inclusion bodies in various regions of the brain. Tau filaments, also referred to as fibrils, represent pathological hallmarks of Alzheimer's disease and more than twenty other neurodegenerative disorders. Tau aggregation is mediated by microtubule binding repeat regions, with different tauopathies classified according to whether they contain three-repeat (3R) Tau isoforms, four-repeat (4R) Tau isoforms, or mixtures of both. Pick's disease represents a 3R tauopathy, while progressive supranuclear palsy and corticobasal degeneration represent 4R tauopathies, and Alzheimer's disease and chronic traumatic encephalopathy involve mixtures of 3R and 4R isoforms. Cryo-electron microscopy structures of Tau amyloid filaments isolated from patients with different tauopathies have revealed structural diversity, with disease-specific fibril folds even within exclusive 3R, 4R, or mixed tauopathies. These filaments can display prion-like transmission behavior, where pre-existing filaments catalyze recruitment of Tau monomers, driving propagation and contributing to spread of pathology through homotypic interactions in which newly incorporated monomers stack in a parallel in-register beta-sheet structure.

Tau shares sequence similarity with microtubule-associated proteins MAP2 and MAP4, which regulate microtubule stability and organelle transport in neuronal and non-neuronal cells. The sequence similarity between these proteins originates from three or four microtubule binding repeat regions present in MAP2 and MAP4 respectively. MAP proteins have been observed as part of the Tau interactome in neurofibrillary tangles isolated from Alzheimer's disease brain tissue, and changes in MAP expression have been documented during disease progression.

Various approaches have been explored for modulating Tau fibrillization, including small molecules, short peptides, and antibodies. Small molecules and peptides have limited contact surfaces with fibrils and decreased conformational adaptability. Antibodies present challenges in distinguishing between different fibril conformations, have large molecular weights, and involve substantial development costs.

### BRIEF DESCRIPTION OF FIGURES

Non-limiting and non-exhaustive examples are described with reference to the following figures.

FIG. 1A illustrates an illustrative sequence alignment between MAP2 and 3R Tau proteins, in accordance with various embodiments disclosed herein.

FIG. 1B illustrates an illustrative sequence alignment between MAP2 and 4R Tau proteins, in accordance with various embodiments disclosed herein.

FIG. 2A illustrates an illustrative docking of MAP2 on disease-associated Tau fibril ends and surfaces, in accordance with various embodiments disclosed herein.

FIG. 2B illustrates an illustrative molecular dynamics simulation showing distance measurements between MAP2 and Tau fibril chains over time, in accordance with various embodiments disclosed herein.

FIG. 2C illustrates an illustrative interchain energy analysis for MAP2-Tau fibril complexes, in accordance with various embodiments disclosed herein.

FIG. 3A illustrates an illustrative Thioflavin T fluorescence data from RT-QuIC reactions of Pick's disease brain extracts with and without MAP2, in accordance with various embodiments disclosed herein.

FIG. 3B illustrates an illustrative quantitative analysis of fibril formation in Pick's disease brain extracts in the presence of MAP2, in accordance with various embodiments disclosed herein.

FIG. 3C illustrates an illustrative electron microscopy images of fibril formation from Pick's disease brain extracts, in accordance with various embodiments disclosed herein.

FIG. 4A illustrates an illustrative Thioflavin T fluorescence data from RT-QuIC reactions of Alzheimer's disease brain extracts with and without MAP2, in accordance with various embodiments disclosed herein.

FIG. 4B illustrates an illustrative quantitative analysis of fibril formation in Alzheimer's disease brain extracts in the presence of MAP2, in accordance with various embodiments disclosed herein.

FIG. 4C illustrates an illustrative electron microscopy images of fibril formation from Alzheimer's disease brain extracts, in accordance with various embodiments disclosed herein.

FIG. 5A illustrates an illustrative Thioflavin T fluorescence data from RT-QuIC reactions of progressive supranuclear palsy brain extracts with and without MAP2, in accordance with various embodiments disclosed herein.

FIG. 5B illustrates an illustrative quantitative analysis of fibril formation in progressive supranuclear palsy brain extracts in the presence of MAP2, in accordance with various embodiments disclosed herein.

FIG. 5C illustrates an illustrative electron microscopy images of fibril formation from progressive supranuclear palsy brain extracts, in accordance with various embodiments disclosed herein.

FIG. 6A illustrates an illustrative sequence alignment between MAP4 and 3R/4R Tau proteins, in accordance with various embodiments disclosed herein.

FIG. 6B illustrates an illustrative homology-based modeling of MAP4 into disease-associated Tau folds, in accordance with various embodiments disclosed herein.

FIG. 7 illustrates an illustrative saturation mutagenesis map of MAP2 bound to a Tau fibril having an Alzheimer's disease fold, in accordance with various embodiments disclosed herein.

FIG. 8A illustrates an illustrative computational workflow for analyzing inhibition of Tau fibril aggregation by modified proteins, in accordance with various embodiments disclosed herein.

FIG. 8B illustrates an illustrative experimental workflow for analyzing inhibition of Tau fibril aggregation by modified proteins, in accordance with various embodiments disclosed herein.

FIG. 9A illustrates a sequence alignment of repeat regions of 3R/4R Tau with MAP2 and MAP4, in accordance with various embodiments disclosed herein.

FIG. 9B illustrates IUPred disorder prediction analysis for MAP2 and MAP4, in accordance with various embodiments disclosed herein.

FIG. 9C illustrates ab-initio modeling of MAP proteins showing disordered structural nature, in accordance with various embodiments disclosed herein.

FIG. 9D illustrates homology-based modeling of MAP2 into disease-associated Tau folds, in accordance with various embodiments disclosed herein.

FIG. 9E illustrates homology-based modeling of MAP4 into disease-associated Tau folds, in accordance with various embodiments disclosed herein.

FIG. 10 illustrates an illustrative Ramachandran plots and structural analysis parameters for homology modeling of MAP2 and MAP4, in accordance with various embodiments disclosed herein.

FIG. 11A illustrates structural representations of MAP2 and MAP4 docked onto disease-associated Tau fibril folds, in accordance with various embodiments disclosed herein.

FIG. 11B illustrates Ca-RMSD traces showing temporal stability of MAP2 and MAP4 on Tau fibrils, in accordance with various embodiments disclosed herein.

FIG. 11C illustrates binding energy analyses for Tau-Tau, MAP2-Tau, and MAP4-Tau interactions, in accordance with various embodiments disclosed herein.

FIG. 12A illustrates a Ca-RMSD graph for molecular dynamics simulations in an Alzheimer's disease fibril fold, in accordance with various embodiments disclosed herein.

FIG. 12B illustrates structural snapshots from simulation trajectories in an Alzheimer's disease fibril fold, in accordance with various embodiments disclosed herein.

FIG. 13A illustrates a Ca-RMSD graph for molecular dynamics simulations in a Pick's disease fibril fold, in accordance with various embodiments disclosed herein.

FIG. 13B illustrates structural snapshots from simulation trajectories in a Pick's disease fibril fold, in accordance with various embodiments disclosed herein.

FIG. 14A illustrates a Ca-RMSD graph for molecular dynamics simulations in a progressive supranuclear palsy fibril fold, in accordance with various embodiments disclosed herein.

FIG. 14B illustrates structural snapshots from simulation trajectories in a progressive supranuclear palsy fibril fold, in accordance with various embodiments disclosed herein.

FIG. 15A illustrates Thioflavin T fluorescence data for Alzheimer's disease brain extracts with 4R Tau, in accordance with various embodiments disclosed herein.

FIG. 15B illustrates sedimentation analysis for Alzheimer's disease brain extracts with 4R Tau, in accordance with various embodiments disclosed herein.

FIG. 15C illustrates electron microscopy images for Alzheimer's disease brain extracts with 4R Tau, in accordance with various embodiments disclosed herein.

FIG. 16 illustrates an illustrative SDS-PAGE gel images showing sedimentation analysis with Alzheimer's disease seeds and 4R Tau substrate, in accordance with various embodiments disclosed herein.

FIG. 17A illustrates Thioflavin T fluorescence data for Alzheimer's disease brain extracts with 3R Tau, in accordance with various embodiments disclosed herein.

FIG. 17B illustrates sedimentation analysis for Alzheimer's disease brain extracts with 3R Tau, in accordance with various embodiments disclosed herein.

FIG. 17C illustrates electron microscopy images for Alzheimer's disease brain extracts with 3R Tau, in accordance with various embodiments disclosed herein.

FIG. 18 illustrates an illustrative SDS-PAGE gel images showing sedimentation analysis with Alzheimer's disease seeds and 3R Tau substrate, in accordance with various embodiments disclosed herein.

FIG. 19A illustrates Thioflavin T fluorescence data for Pick's disease brain extracts with MAP2 and MAP4, in accordance with various embodiments disclosed herein.

FIG. 19B illustrates sedimentation analysis for Pick's disease brain extracts with MAP2 and MAP4, in accordance with various embodiments disclosed herein.

FIG. 19C illustrates electron microscopy images for Pick's disease brain extracts, in accordance with various embodiments disclosed herein.

FIG. 20 illustrates an illustrative SDS-PAGE gel images showing sedimentation analysis with Pick's disease seeds and 3R Tau substrate, in accordance with various embodiments disclosed herein.

FIG. 21 illustrates an illustrative sedimentation analysis validating a seeding barrier between Pick's disease seeds and 4R Tau protein, in accordance with various embodiments disclosed herein.

FIG. 22A illustrates Thioflavin T fluorescence data for progressive supranuclear palsy brain extracts with MAP2 and MAP4, in accordance with various embodiments disclosed herein.

FIG. 22B illustrates sedimentation analysis for progressive supranuclear palsy brain extracts with MAP2 and MAP4, in accordance with various embodiments disclosed herein.

FIG. 22C illustrates electron microscopy images for progressive supranuclear palsy brain extracts, in accordance with various embodiments disclosed herein.

FIG. 23 illustrates an illustrative SDS-PAGE gel images showing sedimentation analysis with progressive supranuclear palsy seeds and 4R Tau substrate, in accordance with various embodiments disclosed herein.

FIG. 24 illustrates an illustrative SDS-PAGE gel images showing sedimentation analysis validating a seeding barrier between progressive supranuclear palsy seeds and 3R Tau, in accordance with various embodiments disclosed herein.

FIG. 25A illustrates Thioflavin T fluorescence data for control brain samples with 3R Tau, in accordance with various embodiments disclosed herein.

FIG. 25B illustrates sedimentation analysis for control brain samples with 3R Tau, in accordance with various embodiments disclosed herein.

FIG. 26A illustrates Thioflavin T fluorescence data for control brain samples with 4R Tau, in accordance with various embodiments disclosed herein.

FIG. 26B illustrates sedimentation analysis for control brain samples with 4R Tau, in accordance with various embodiments disclosed herein.

FIG. 27 illustrates an illustrative spontaneous aggregation assays for 3R Tau, 4R Tau, MAP2, and MAP4 alone, in accordance with various embodiments disclosed herein.

FIG. 28A illustrates isothermal titration calorimetry kinetics showing heat signatures of Tau seeding, in accordance with various embodiments disclosed herein.

FIG. 28B illustrates total heat analysis bar graphs for Tau seeding reactions, in accordance with various embodiments disclosed herein.

FIG. 29 illustrates an illustrative Thioflavin T fluorescence measurements from isothermal titration calorimetry endpoint analysis, in accordance with various embodiments disclosed herein.

FIG. 30 illustrates an illustrative isothermal titration calorimetry data for MAP2 and MAP4 interactions with Tau monomers, in accordance with various embodiments disclosed herein.

FIG. 31A illustrates fluorescent microscopy images of cells transfected with seeds, in accordance with various embodiments disclosed herein.

FIG. 31B illustrates quantitative analysis of puncta formation, in accordance with various embodiments disclosed herein.

FIG. 32 illustrates an illustrative fluorescent microscopy images of cells transfected with a second set of disease-derived seeds, in accordance with various embodiments disclosed herein.

FIG. 33 illustrates an illustrative fluorescent microscopy images of cells transfected with a third set of disease-derived seeds, in accordance with various embodiments disclosed herein.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

According to an aspect of the present disclosure, a polypeptide is provided. The polypeptide may include a polypeptide including the amino acid sequence set forth as SEQ ID NO: 1 corresponding to MAP2, a polypeptide including the amino acid sequence set forth as SEQ ID NO: 2 corresponding to MAP4, or a sequence variant of the foregoing sharing 20-99% sequence similarity with the structured core regions of pathological Tau filaments and retaining functional activity in reducing Tau aggregation or Tau seeding activity. The polypeptide is capable of binding to an end of a Tau fibril and forming a terminal layer cap that decreases Tau aggregation and/or Tau seeding activity, where the polypeptide does not self-propagate or template further growth of Tau or MAP monomers, and in some aspects may include a Tau protein variant differing from native Tau by one or more amino acids (for example, one or two residues) that shares sufficient sequence similarity to engage the fibril end and acts as a terminal cap without supporting further layer stacking or elongation, thereby blocking access for incoming Tau monomers and preventing fibril elongation.

According to other aspects of the present disclosure, the polypeptide may include one or more of the following features. The variant may exhibit between about 20 percent and about 99 percent sequence identity to SEQ ID NO:1 or SEQ ID NO:2 and may maintain the ability to inhibit Tau aggregation. Inhibition of Tau aggregation may be at least about 20 percent, 50 percent, or 70 percent as determined in a Thioflavin T assay, Tau biosensor seeding assay, electron microscopy particle quantification, atomic force microscopy analysis, or in vivo model. The polypeptide may further include a fusion partner, tag, half-life extending element, or brain delivery module such as an Fc region, albumin binding domain, blood-brain barrier shuttle peptide, transferrin receptor targeting domain, apolipoprotein mimetic sequence, FcRn enhancing mutation, or cell penetrating peptide. The polypeptide may be modified by phosphorylation, acetylation, PEG conjugation, glycoengineering, cyclization, stapling, or lipid attachment while retaining Tau inhibitory function. The polypeptide may include a contiguous fragment of at least 2 amino acids, such as at least 5, 10, 15, 20, 25, or 30 amino acids, or any integer therebetween, derived from SEQ ID NO:1 or SEQ ID NO:2, provided that the fragment retains measurable Tau inhibitory activity. If such a fragment binds to the fibril end without supporting elongation, the fragment may serve as a cap that blocks access for incoming Tau monomers. The inhibitory activity of such fragments may be tested using Thioflavin T fluorescence assays, sedimentation assays, electron microscopy imaging, or cell-based puncta assays as described herein. The polypeptide may be engineered as a consensus sequence, chimeric construct, humanized form, or de-immunized variant reducing predicted human T cell epitopes but still active against Tau aggregation. The polypeptide may associate with an aggregation prone region of Tau and may interfere with nucleation and/or elongation steps of fibril formation. The polypeptide may decrease prion-like cell-to-cell propagation of Tau seeds. The polypeptide may be configured as a dimeric, multimeric, scaffold displayed, or Fc fused construct so as to increase binding affinity towards monomeric Tau. The polypeptide may be fused to an antibody or antibody fragment that binds a receptor mediating blood brain barrier transport to facilitate central nervous system uptake.

According to another aspect of the present disclosure, a pharmaceutical composition is provided. The pharmaceutical composition includes the polypeptide described above in combination with a pharmaceutically acceptable carrier, excipient, or diluent suitable for administration.

According to other aspects of the present disclosure, the pharmaceutical composition may include one or more of the following features. The pharmaceutical composition may be formulated for administration by intravenous, subcutaneous, intrathecal, intracerebroventricular, intranasal, or convection enhanced delivery routes. The pharmaceutical composition may further include a nanoparticle, liposomal carrier, exosome-based system, polymeric depot, hydrogel formulation, or implantable device for sustained release. The pharmaceutical composition may be provided in a dosage form appropriate for weekly, biweekly, monthly, or quarterly dosing intervals. The polypeptide may include SEQ ID NO:1 or SEQ ID NO:2 and the carrier may include a buffered solution containing a stabilizer, surfactant, tonicity adjusting agent, or combination thereof suitable for parenteral delivery.

According to another aspect of the present disclosure, a method for treating a tauopathy in a subject is provided. The method includes administering to the subject an effective amount of the polypeptide described above sufficient to reduce Tau associated pathology.

According to other aspects of the present disclosure, the method may include one or more of the following features. The method may further include monitoring therapeutic response by measuring a reduction in phosphorylated Tau levels, a reduction in Tau seeding activity, or an improvement in cognitive or functional assessment relative to baseline. The method may further include administering cognitive rehabilitation or neuromodulation therapy in conjunction with the polypeptide treatment. The tauopathy may be any tauopathy including, but not limited to, Alzheimer's disease, progressive supranuclear palsy, corticobasal degeneration, Pick's disease, argyrophilic grain disease, globular glial tauopathy, frontotemporal lobar degeneration with Tau, chronic traumatic encephalopathy, MAPT mutation associated frontotemporal dementia, or mixed pathologies involving Tau. The subject may be identified as having elevated genetic risk or biomarker-based risk for a tauopathy. Dosing may be adjusted according to pharmacokinetic parameters, anti-drug antibody titers, or biomarker response in the subject. Treatment may produce clinically meaningful improvement in cognitive or functional outcome relative to baseline measurement. Administration may be intravenous, subcutaneous, intrathecal, intracerebroventricular, intranasal, or via convection enhanced delivery. The subject may exhibit a reduction in Tau PET imaging signal, a reduction in cerebrospinal fluid pTau181 or pTau217 levels, or a decrease in Tau seeding activity following treatment.

According to another aspect of the present disclosure, an isolated nucleic acid molecule encoding the polypeptide described above is provided. An expression vector including said nucleic acid is also provided. A host cell containing said vector and capable of expressing the encoded polypeptide is also provided.

According to other aspects of the present disclosure, the expression vector may include one or more of the following features. The coding sequence may be operably linked to a secretion signal sequence or a blood brain barrier shuttle sequence enabling central nervous system delivery.

According to another aspect of the present disclosure, a method for reducing Tau aggregation, growth, or seeding in a biological or biochemical sample containing Tau is provided. The method includes contacting the sample with the polypeptide described above under conditions in which Tau aggregation is measurably reduced relative to a control sample.

According to another aspect of the present disclosure, a method for preparing a medicament for prevention or treatment of a tauopathy is provided. The method includes admixing the polypeptide described above with a pharmaceutically acceptable carrier to obtain a formulated therapeutic composition.

According to other aspects of the present disclosure, the method may include one or more of the following features. The medicament may be filled into a vial, prefilled syringe, or autoinjector device and labeled for use in treatment of a tauopathy.

According to another aspect of the present disclosure, a method for treating a tauopathy is provided. The method includes administering to a subject the expression vector described above such that the polypeptide is expressed within the central nervous system.

According to another aspect of the present disclosure, a method for producing the polypeptide described above is provided. The method includes expressing the polypeptide in a prokaryotic or eukaryotic host cell and purifying the expressed product using affinity chromatography, ion exchange chromatography, size exclusion chromatography, or combinations thereof to obtain therapeutic grade purity.

According to another aspect of the present disclosure, a kit is provided. The kit includes the polypeptide described above or the expression vector described above together with instructions for use in treating a tauopathy. The kit may optionally include a diagnostic assay for identifying suitable patients.

According to other aspects of the present disclosure, the polypeptide may include one or more of the following features. The selected variant may demonstrate improved stability, extended serum half-life, enhanced blood brain barrier penetration, or reduced immunogenic potential relative to SEQ ID NO:1 or SEQ ID NO:2.

According to another aspect of the present disclosure, a method for detecting aggregated or seeding competent Tau species in a biological sample is provided. The method includes contacting the sample with the polypeptide described above immobilized on a solid support, allowing binding of Tau species present in the sample, and detecting bound Tau. The immobilized polypeptide functions as a capture reagent that binds to aggregated Tau species present in the biological sample due to the affinity between the polypeptide and fibrillar Tau ends, thereby enabling pullout and enrichment of aggregated Tau from complex biological matrices. The immobilized polypeptide functions analogously to an antibody in that it may capture, detect, and inhibit target molecules, but with the advantage that the intrinsically disordered nature of the polypeptide enables easier manipulation through site-directed mutagenesis in the core region, allowing rapid and cost-efficient tailoring of the polypeptide to enhance target range, selectivity, and interactive surface area. The level of bound Tau is indicative of the presence or extent of a tauopathy.

According to other aspects of the present disclosure, the method may include one or more of the following features. The biological sample may include cerebrospinal fluid, plasma, serum, whole blood, saliva, urine, or a brain derived extracellular vesicle preparation. The immobilized polypeptide may selectively capture oligomeric or fibrillar Tau relative to monomeric Tau.

According to another aspect of the present disclosure, a diagnostic assay is provided. The diagnostic assay includes the polypeptide described above configured as a capture reagent in a sandwich format. The diagnostic assay further includes a detection antibody or labeled binding partner recognizing Tau. The assay quantifies Tau aggregates or Tau seeding activity in a biofluid sample obtained from a subject.

According to another aspect of the present disclosure, a method of stratifying a subject for risk or progression of a tauopathy is provided. The method includes incubating a biofluid sample from the subject with the polypeptide described above under conditions sufficient to bind aggregation prone Tau species, measuring the extent of binding or capture, and correlating increased binding relative to a reference control with elevated disease burden or risk.

According to another aspect of the present disclosure, a diagnostic kit is provided. The diagnostic kit includes the polypeptide described above immobilized on a plate, bead, membrane, or microfluidic surface. The diagnostic kit may optionally include reagents for detection of captured Tau including labeled antibodies, fluorescent probes, enzymatic substrates, or amplification reagents. The diagnostic kit includes instructions for use in analyzing Tau species from cerebrospinal fluid or peripheral biofluid samples.

According to some embodiments of the present disclosure, a polypeptide capable of inhibiting Tau fibril elongation is provided where the polypeptide includes an amino acid sequence exhibiting between 20% and 99% sequence identity to SEQ ID NO: 1 corresponding to MAP2 or SEQ ID NO:2 corresponding to MAP4, and where the polypeptide is capable of binding to an end of a Tau fibril and forming a terminal layer cap that reduces Tau aggregation or Tau seeding activity, and the polypeptide does not self-propagate or template further growth of Tau monomers or MAP monomers onto the fibril.

In various embodiments, the amino acid sequence includes one or more amino acid substitutions relative to SEQ ID NO:1 or SEQ ID NO:2, and the polypeptide retains functional activity in reducing Tau aggregation or Tau seeding activity. In some embodiments, the polypeptide includes a contiguous fragment of at least 2 amino acids derived from SEQ ID NO: 1 or SEQ ID NO:2 that retains measurable Tau inhibitory activity. In some embodiments, the polypeptide decreases prion-like cell-to-cell propagation of Tau seeds. In some embodiments, the polypeptide is capable of adopting a disease-associated Tau fold conformation upon binding to the end of the Tau fibril. In some embodiments, the polypeptide inhibits aggregation of both three-repeat (3R) Tau isoforms and four-repeat (4R) Tau isoforms, including wild-type Tau and pathogenic Tau variants characterized by single amino acid substitutions or deletions associated with frontotemporal dementia and parkinsonism linked to chromosome 17 (FTDP-17). In some embodiments, the polypeptide further includes a fusion partner selected from the group consisting of an Fc region, an albumin binding domain, a blood-brain barrier shuttle peptide, a transferrin receptor targeting domain, and a cell penetrating peptide. In some embodiments, the polypeptide is formulated as a pharmaceutical composition with a pharmaceutically acceptable carrier. In some embodiments, the pharmaceutical composition is formulated for at least one of intravenous, subcutaneous, intrathecal, intracerebroventricular, intranasal, or convection-enhanced delivery.

According to various embodiments of the present disclosure, methods for treating a tauopathy in a subject are provided, where the methods include administering to the subject a therapeutically effective amount of a polypeptide including an amino acid sequence exhibiting between 20% and 99% sequence identity to SEQ ID NO: 1 corresponding to MAP2 or SEQ ID NO: 2 corresponding to MAP4, where the polypeptide binds to ends of Tau fibrils in the subject and reduces Tau-associated pathology.

In various embodiments, the tauopathy includes at least one of Alzheimer's disease, progressive supranuclear palsy, corticobasal degeneration, Pick's disease, argyrophilic grain disease, globular glial tauopathy, frontotemporal lobar degeneration with Tau, chronic traumatic encephalopathy, or frontotemporal dementia associated with MAPT mutation. In some embodiments, the polypeptide is modified by at least one of polyethylene glycol (PEG) conjugation, glycoengineering, cyclization, stapling, or lipid attachment, and the polypeptide retains Tau inhibitory function following the modification. In some embodiments, the method can further include monitoring therapeutic response by measuring at least one of a reduction in phosphorylated Tau levels, a reduction in Tau seeding activity, a reduction in Tau positron emission tomography (PET) imaging signal, or an improvement in a cognitive or functional assessment relative to baseline. In some embodiments, the polypeptide is fused to an antibody or antibody fragment that binds a receptor mediating blood-brain barrier transport, facilitating central nervous system uptake. In some embodiments, the polypeptide is engineered as a consensus sequence, chimeric construct, humanized form, or de-immunized variant that reduces predicted human T cell epitopes while retaining activity against Tau aggregation.

According to various aspects of the present disclosure, methods for detecting aggregated Tau species in a biological sample are provided. The methods include contacting the biological sample with a polypeptide immobilized on a solid support, where the polypeptide includes an amino acid sequence exhibiting between 20% and 99% sequence identity to SEQ ID NO: 1 corresponding to MAP2 or SEQ ID NO:2 corresponding to MAP4, binding aggregated Tau species present in the biological sample to the immobilized polypeptide, and detecting bound Tau species, where a level of bound Tau species indicates the presence or extent of a tauopathy in a subject from which the biological sample was obtained.

In various embodiments, the biological sample includes at least one of cerebrospinal fluid, plasma, serum, whole blood, saliva, urine, or a brain-derived extracellular vesicle preparation. In some embodiments, the immobilized polypeptide selectively captures oligomeric or fibrillar Tau relative to monomeric Tau. In some embodiments, the method further includes a detection antibody or labeled binding partner recognizing Tau, where the immobilized polypeptide and the detection antibody or labeled binding partner are configured in a sandwich format that quantifies Tau aggregates or Tau seeding activity in the biological sample. In some embodiments, the method further includes contacting the biological sample with a plurality of polypeptide variants, each variant including an amino acid sequence exhibiting between 20% and 99% sequence identity to SEQ ID NO:1 or SEQ ID NO:2, where the plurality of polypeptide variants exhibit differential binding to Tau fibril conformations associated with different tauopathies, and where a pattern of binding across the plurality of polypeptide variants is used to distinguish between tauopathies in the subject from which the biological sample was obtained.

As used herein, the terms “sequence similarity” and “sequence identity” may be used interchangeably to refer to the degree of relatedness between amino acid sequences, such as between the polypeptide constructs described herein and the structured core regions of pathological Tau filament [Inventors please update]s.

As used herein, the term “Tau fibril elongation” is also referred to as “Tau aggregation” or “Tau fibril aggregation.” It represents the growth of Tau monomers onto an existing seed. Spontaneous aggregation involves both nucleation and elongation. When a seed or nucleus is already present, aggregation is dominated by elongation. At very high protein concentrations, nucleation may dominate the process, which may be analogous to protein crystallography where many small crystals may form due to parallel nucleation events occurring simultaneously.

As used herein, the terms “three-repeat” or “3R” when referring to Tau isoforms indicate Tau protein variants containing three microtubule binding repeats. The terms “four-repeat” or “4R” when referring to Tau isoforms indicate Tau protein variants containing four microtubule binding repeats. The abbreviations “3R” and “4R” may be used interchangeably with “three-repeat” and “four-repeat” respectively throughout this disclosure.

As used herein, the term “pathogenic Tau variant” refers to Tau proteins bearing one or more amino acid substitutions or deletions relative to wild-type Tau that are associated with hereditary tauopathies, including variants associated with frontotemporal dementia and parkinsonism linked to chromosome 17 (FTDP-17). Examples of such variants include, but are not limited to, P301S, P301L, V337M, R406W, G272V, N279K, and other MAPT gene mutations that cause or increase susceptibility to tauopathy.

As used herein, the term “glycoengineering” refers to the addition or redesign of carbohydrate moieties on MAP2 or MAP4 derived proteins or peptides. Glycoengineering may provide beneficial pharmacological or pharmacokinetic effects. In some cases, glycan binding may reduce non-specific hydrophobic collapse of MAP proteins. In some cases, glycation may protect proteins or peptides from proteolysis and may improve half-life and distribution. In some aspects, glycation at certain positions may help prevent derived peptides from self-aggregation.

As used herein, the term “positron emission tomography” or “PET” refers to a neuroimaging technique that may be used to visualize and measure Tau pathology in the brain. PET imaging using Tau-specific radiotracers enables detection and quantification of Tau deposits in living subjects. The terms “positron emission tomography” and “PET” may be used interchangeably throughout this disclosure, and “Tau PET imaging” refers to PET imaging performed with radiotracers that bind to aggregated Tau species.

Tau filaments represent pathological hallmarks of Alzheimer's disease and more than twenty other fatal neurodegenerative disorders collectively referred to as tauopathies. Tauopathies include Alzheimer's disease, progressive supranuclear palsy, corticobasal degeneration, Pick's disease, argyrophilic grain disease, globular glial tauopathy, frontotemporal lobar degeneration with Tau, chronic traumatic encephalopathy, and frontotemporal dementia associated with MAPT mutation. In Alzheimer's disease, the presence of amyloid-beta plaques and Tau neurofibrillary tangles characterizes the condition. Understanding mechanisms behind pathological aggregation of Tau protein may provide approaches for diagnosing and treating tauopathy-based neurodegenerative disorders.

Tau filaments grow in a template-assisted manner, whereby Tau monomers stack on top of other Tau monomers in the fibril in parallel and in-register fashion. In this process, incoming Tau monomers may be molded into the shape of the template. The elongation of Tau filaments, mediated by a central core region in the protein, facilitates the propagation and spread of Tau pathology throughout the human brain to result in various types of neurodegenerative conditions. Stopping this propagation process may hold approaches to combating Tau pathology.

Currently known inhibitors of Tau fibrillization include small molecules, short peptides, and antibodies. Small molecules and peptides present limitations including limited contact surfaces with the fibril, decreased conformational adaptability, and increased probability for off-target interactions. These limitations reduce the functionality of small molecules and peptides as Tau fibrillization inhibitors. While antibodies may target fibrillization, distinguishing between different fibril conformations presents challenges in antibody-based approaches. Most antibodies detect small sequence-dependent epitopes, and antibodies that detect conformations remain scarce. Antibodies also have large molecular weights and are expensive to generate. Developing a diverse set of antibody molecules to achieve high diagnostic accuracy remains difficult and costly.

Prior techniques using inhibitors such as MAP2 to cap synthetic Tau fibrils employed heparin as a cofactor. Fibrils assembled using heparin as a cofactor have a different structure from fibrils isolated from disease tissue obtained from clinical patients. In prior techniques with cofactors, inhibitor proteins appeared in the pellet and formed amorphous aggregates in the presence of heparin, complicating interpretation of results. The inhibition of Tau aggregation may be performed in the absence of cofactors such as heparin or dextran sulfate, thereby preserving the native structure of disease-associated fibrils and avoiding amorphous aggregation of inhibitor proteins.

In contrast to small molecule and antibody approaches, homology-based protein molecules may engage with the growing fibril end via homology-based interactions spanning the entire cross-section of the fibril tip. The resulting interactive surface extends across the entire cross-section of the fibril tip, providing extensive contact compared to the limited contact surfaces of small molecules and antibodies. Homology-based protein molecules may be manipulated by mutagenesis to enhance binding affinity. Since Tau fibrils involved in different tauopathies present structures with varying cross-sectional packing interactions due to different meanderings of the Tau backbone, homology-based protein molecules may be mutated to interact with select conformers. Such molecules may therefore serve as a basis for development of targeted therapies and diagnostics for tauopathies.

The endogenous context of MAPs in disease provides additional rationale for investigating MAP-based approaches to modulating Tau pathology. MAPs have been observed as part of the Tau interactome in neurofibrillary tangles isolated from Alzheimer's disease brain tissue in humans. Post-mortem brains from Alzheimer's disease individuals have shown significant reduction in MAP2 expression in the CA hippocampal regions and in the dentate gyrus. A significant reduction in MAP2-immunopositive neurons has also been observed in Alzheimer's disease-derived neuronal cultures compared to non-cognitively impaired-derived cultures. These observations suggest that changes in MAP expression may be associated with progression of tauopathies, and that MAPs may play a role in modulating Tau aggregation processes in vivo.

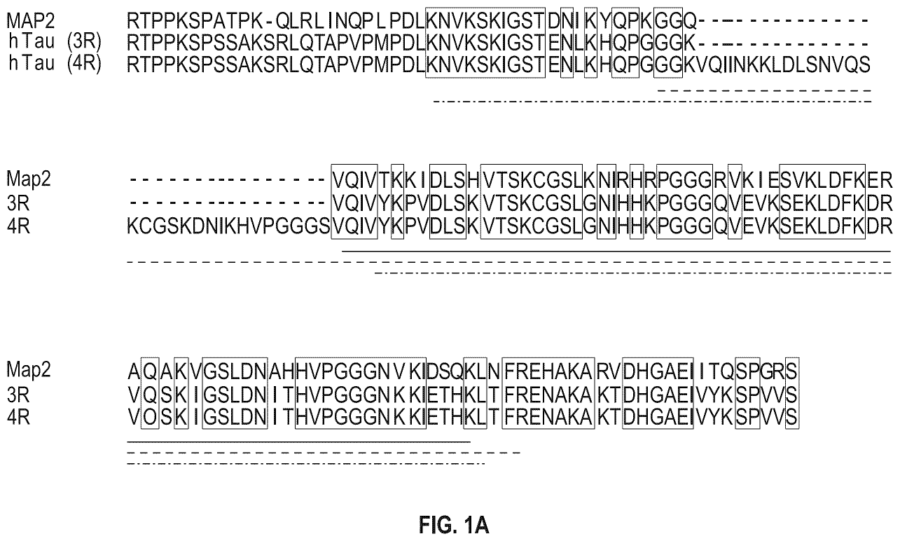

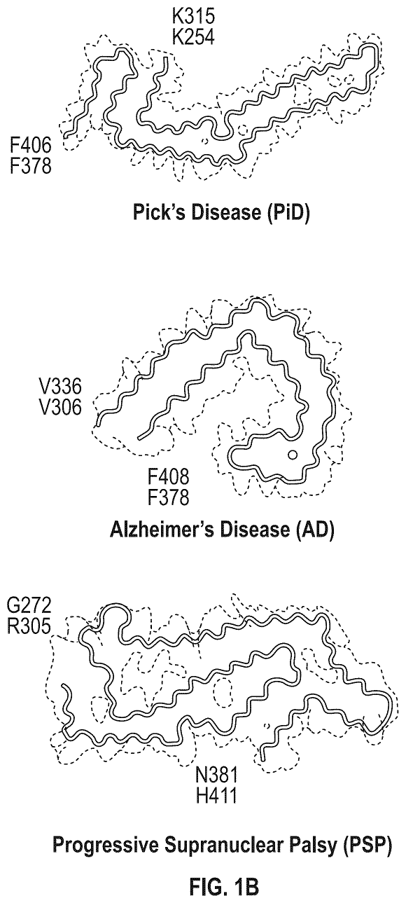

Referring to FIG. 1A, a sequence alignment illustrates regions of similarity between MAP2 and 3R/4R Tau proteins. The alignment shows three sequences arranged vertically, with MAP2 positioned at the top, human Tau 3R (hTau 3R) in the middle, and human Tau 4R (hTau 4R) at the bottom. Each sequence displays amino acid residues in single-letter code format. Shaded regions within the alignment indicate high conservation of amino acids between MAP2 and the Tau variants. The alignment demonstrates that MAP2 shares between 49% and 80% sequence similarity with the structured core region of Tau filaments, with the conserved regions corresponding to microtubule binding repeat domains that form the amyloid core in various disease-associated Tau folds.

With continued reference to FIG. 1A, colored bars positioned below the alignment indicate core regions responsible for Tau aggregation in different tauopathies. Blue bars indicate core regions responsible for Tau aggregation in Pick's disease (PiD). Red bars indicate core regions for Alzheimer's disease (AD). Green bars indicate core regions for progressive supranuclear palsy (PSP) tauopathies. Dashes in the MAP2 and 3R sequences represent gaps in the alignment where residues are absent compared to the 4R Tau sequence. The sequences of MAP2 and Tau may be retrieved from UniProt, and alignments may be performed using Clustal Omega or similar alignment tools.

Referring to FIG. 1B, homology-based modeling of MAP2 into different types of disease-associated Tau folds is illustrated. Three-dimensional alignment of modeled MAP2 chains over Tau fibrils from PiD, AD, and PSP indicates that MAP2 may acquire different types of disease-specific Tau folds. In the PiD fold representation, a MAP2 chain follows the characteristic curved conformation of the Pick's disease-associated Tau fibril structure. In the AD fold representation, a MAP2 chain adopts the meandering path characteristic of the Alzheimer's disease-associated Tau fibril. In the PSP fold representation, a MAP2 chain conforms to the more elongated structure associated with progressive supranuclear palsy Tau fibrils. The gray structures in each representation depict the underlying Tau fibril architecture while the colored chains indicate the modeled MAP2 protein adopting the respective disease-specific conformations.

The homology-based approach provides several key advantages over small molecule and antibody approaches. Because MAP2 shares substantial sequence identity with the Tau core region, MAP2-based constructs may engage with fibril ends through interactions spanning the entire cross-section of the fibril tip, providing extensive contact compared to the limited contact surfaces of small molecules. The conformational adaptability of MAP2 allows the protein to adopt different disease-specific folds, enabling broad-spectrum activity against multiple tauopathies including Alzheimer's disease, Pick's disease, and progressive supranuclear palsy. This broad-spectrum activity may enable development of therapeutic agents effective against multiple tauopathies without requiring disease-specific modifications. The sequence similarity also provides a framework for rational design of variants through site-directed mutagenesis to enhance binding affinity or selectivity for particular fibril conformers. Additionally, the reproducibility of inhibitory activity across multiple patient-derived biological samples supports the robustness of the approach for both therapeutic and diagnostic applications. Homology modeling may be performed using SwissModel workspace or similar tools, and three-dimensional alignments may be generated using PyMol or equivalent molecular graphics software.

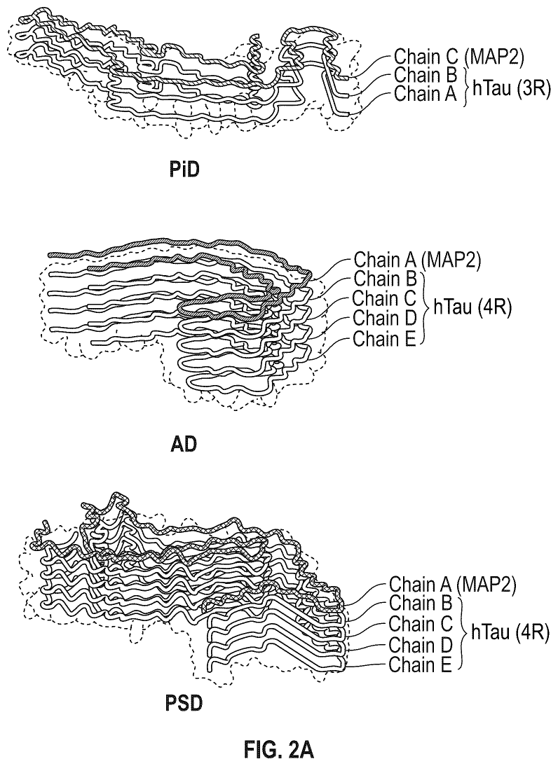

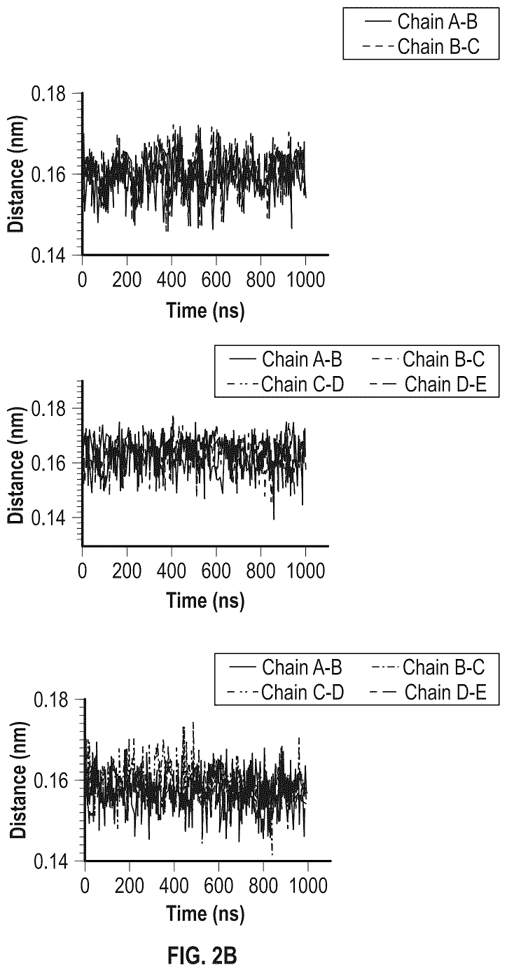

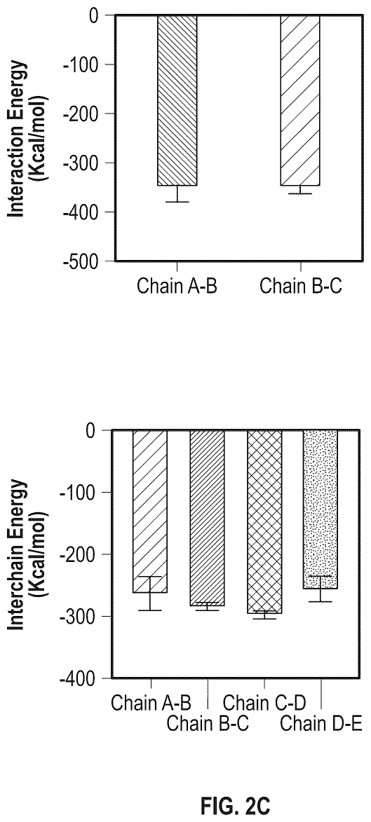

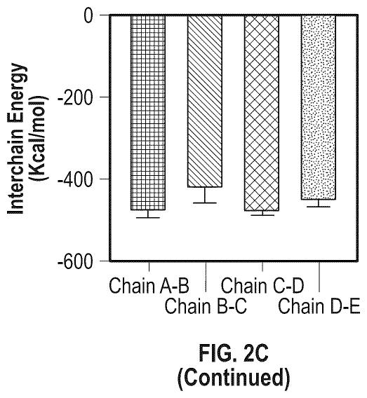

In the PiD structure shown in FIG. 2A, a MAP2 chain depicted in blue is docked onto a three-layered Tau fibril. Chain C is designated as MAP2, while Chains A and B are designated as hTau 3R. The MAP2 chain follows the meandering path characteristic of the Pick's disease-associated Tau fold, demonstrating that MAP2 may adopt a conformation complementary to the 3R Tau fibril surface.

With continued reference to FIG. 2A, the AD structure shows a MAP2 chain depicted in red docked onto a four-layered Tau fibril. Chain A is designated as MAP2, while Chains B, C, D, and E are designated as hTau 4R. The MAP2 chain traces along the curved conformation characteristic of the Alzheimer's disease-associated Tau fold, indicating that MAP2 may engage with the fibril end through complementary structural interactions.

In the PSP structure shown in FIG. 2A, a MAP2 chain depicted in green is docked onto a four-layered Tau fibril. Chain A is designated as MAP2, while Chains B, C, D, and E are designated as hTau 4R. The MAP2 chain adopts the more elongated conformation associated with progressive supranuclear palsy Tau fibrils, demonstrating the conformational adaptability of MAP2 to different disease-specific fibril architectures.

The docking analysis may be performed using protein-protein docking protocols such as ClusPro. The ClusPro protein-protein docking protocol evaluates binding poses and identifies favorable interaction configurations between MAP2 and Tau fibril structures. The polypeptide may be validated using protein-protein docking protocols such as ClusPro to assess binding potential and structural complementarity with target fibril conformations.

The structural representations in FIG. 2A demonstrate that MAP2 may engage with fibril ends from multiple tauopathies through conformational adaptation. The ability of MAP2 to dock onto fibril surfaces from PiD, AD, and PSP indicates broad-spectrum binding potential across different disease-associated Tau folds. Stable engagement with fibril surfaces may enable MAP2-based constructs to function as fibril caps that prevent recruitment of additional Tau monomers.

Referring to FIG. 2B, molecular dynamics simulations of docked MAP2-Tau fibril complexes show distance measurements between protein chains over time. FIG. 2B contains three separate graphs arranged vertically, each displaying distance in nanometers on the vertical axis ranging from approximately 0.14 to 0.18 nm, and time in nanoseconds on the horizontal axis ranging from 0 to 1000 ns. The simulations assess binding stability of MAP2 on Tau fibrils from three different tauopathies over a microsecond timescale.

The top graph in FIG. 2B shows distance measurements for a PiD fibril complex. Two traces are displayed, representing Chain A-B in black and Chain B-C in blue. The Chain B-C trace illustrates the distance between MAP2 and the adjacent Tau layer in the PiD fibril complex. Throughout the 1000 nanosecond simulation period, the distance values fluctuate around approximately 0.16 nm, indicating that MAP2 maintains stable association with the neighboring Tau monomer.

With continued reference to FIG. 2B, the middle graph displays distance measurements for an AD fibril complex. Four traces are shown, representing Chain A-B in black, Chain B-C in red, Chain C-D in black, and Chain D-E in black. The Chain B-C trace in red represents the distance between MAP2 and the adjacent Tau layer in the Alzheimer's disease fibril complex. The distance values for the MAP2-Tau interface remain comparable to the Tau-Tau distances represented by the other traces, with values fluctuating around approximately 0.16 nm throughout the simulation.

The bottom graph in FIG. 2B shows distance measurements for a PSP fibril complex. Four traces are displayed, representing Chain A-B in black, Chain B-C in green, Chain C-D in black, and Chain D-E in black. The Chain B-C trace in green illustrates the distance between MAP2 and the adjacent Tau layer in the progressive supranuclear palsy fibril complex. The distance measurements demonstrate that MAP2 maintains approximately 0.16 nm distance from the neighboring Tau monomer, comparable to the Tau-Tau monomer distances shown in the other traces.

The polypeptide may be validated using molecular dynamics simulations at microsecond scale to assess binding stability. The simulation outcomes from distance calculations indicate that MAP2 maintains approximately 0.16 nm distance from the neighboring Tau monomer throughout the entire simulation period. The Tau-Tau monomer distances in the fibril complexes also measure approximately 0.16 nm, indicating that MAP2-Tau interactions exhibit comparable spacing to native Tau-Tau interactions within the fibril structure. The polypeptide maintains approximately 0.16 nm distance from neighboring Tau monomer during molecular dynamics simulations, indicating stable binding to the fibril surface.

The stable binding demonstrated in FIG. 2B indicates that MAP2-based constructs may remain engaged with fibril ends for durations sufficient to interfere with Tau monomer recruitment. The comparable distance measurements between MAP2-Tau and Tau-Tau interfaces suggest that MAP2 integrates into the fibril structure in a manner similar to native Tau monomers, enabling effective capping of the fibril end. The molecular dynamics simulations across PiD, AD, and PSP fibril complexes demonstrate stable binding to fibril surfaces from multiple tauopathies.

Referring to FIG. 2C, three bar graphs show interchain energy analyses results for MAP2 in units of kcal/mol. The interchain energy analyses assess binding affinity between MAP2 and Tau layers compared to native Tau-Tau layer interactions within fibril complexes from three different tauopathies. The binding energy analyses were performed using MM-PBSA (Molecular Mechanics energies combined with the Poisson-Boltzmann Surface Area) based calculations to quantify interaction strengths between adjacent protein layers in the docked fibril complexes.

The top graph in FIG. 2C displays interaction energy measurements for a PiD fibril complex. Two chain pairs are shown, with Chain A-B displayed in black and Chain B-C displayed in blue. The Chain B-C measurement represents the MAP2-Tau interaction energy, while the Chain A-B measurement represents a Tau-Tau interaction energy. Both bars extend to approximately −300 to −350 kcal/mol, indicating that MAP2 binds to the adjacent Tau layer with affinity comparable to native Tau-Tau interactions within the Pick's disease fibril structure. Error bars are visible on each measurement, indicating variability in the calculated binding energies.

With continued reference to FIG. 2C, the middle graph shows interchain energy measurements for an AD fibril complex. Four chain pairs are displayed, labeled Chain A-B, Chain B-C, Chain C-D, and Chain D-E. Chain A-B is shown in red and represents the MAP2-Tau interaction, while the remaining chain pairs shown in black and gray represent Tau-Tau interactions within the Alzheimer's disease fibril structure. The binding energy values range from approximately −200 to −300 kcal/mol across the chain pairs. The MAP2-Tau interaction represented by Chain A-B exhibits binding energy comparable to or higher than the Tau-Tau layer interactions, indicating that MAP2 engages with the AD fibril surface with affinity sufficient to compete with native Tau monomer recruitment.

The bottom graph in FIG. 2C displays interchain energy measurements for a PSP fibril complex. Four chain pairs are shown, with Chain A-B displayed in green representing the MAP2-Tau interaction, and Chain B-C, Chain C-D, and Chain D-E displayed in black, gray, and light gray representing Tau-Tau interactions. The binding energy values range from approximately −300 to −500 kcal/mol, with the PSP complex exhibiting higher overall binding energies compared to the AD complex. The MAP2-Tau interaction demonstrates binding energy comparable to the Tau-Tau layer interactions within the progressive supranuclear palsy fibril structure. Error bars are present on each bar, indicating measurement variability across the calculated binding energies.

The polypeptide demonstrates binding energy comparable to or higher than Tau-Tau layer interactions in fibril complexes across all three tauopathies examined. In the AD fibril complex, the binding energy for MAP2 with Tau was higher by approximately-2.33 kcal/mol compared to the Tau-Tau layer. In the PiD complex, MAP2 displayed higher binding energy by approximately-2.6 kcal/mol compared to the Tau-Tau layer. In the PSP complex, MAP2 showed binding energy higher by approximately-0.84 kcal/mol compared to the adjacent Tau layer. The comparable or enhanced binding affinities indicate that MAP2-based constructs may effectively compete with Tau monomers for binding sites at fibril ends.

The interchain energy analyses support characterization of MAP2 as a broad-spectrum fibril capper. The comparable binding affinities between MAP2-Tau and Tau-Tau interactions across PiD, AD, and PSP fibril complexes indicate that MAP2 may engage with fibril surfaces from multiple disease-associated Tau folds with sufficient affinity to interfere with fibril elongation. The binding energy data combined with the distance measurements from molecular dynamics simulations provide complementary evidence that MAP2 maintains stable, high-affinity interactions with Tau fibril surfaces from diverse tauopathies.

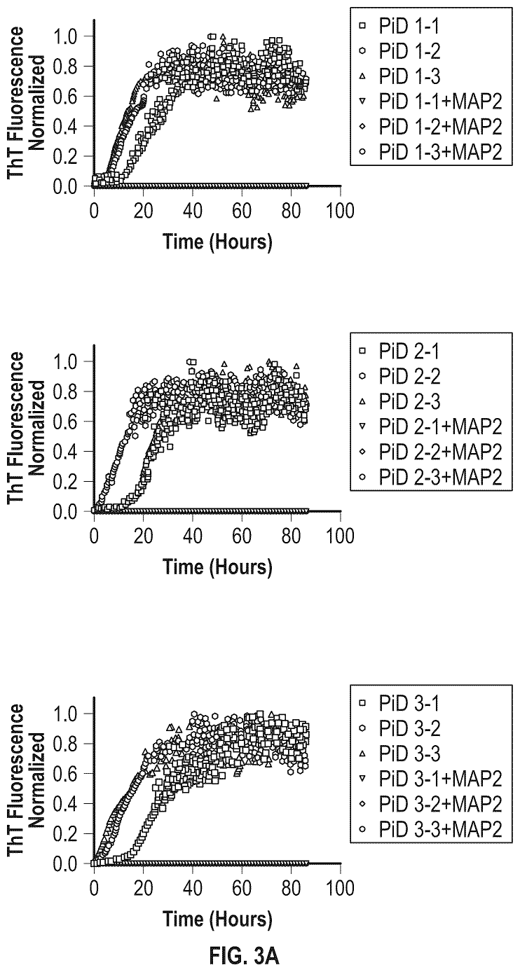

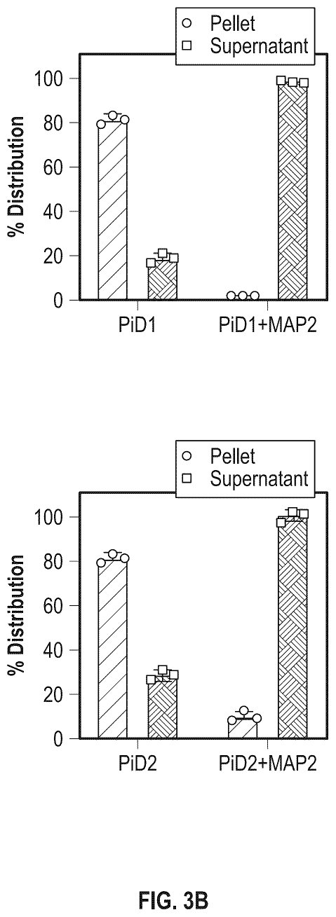

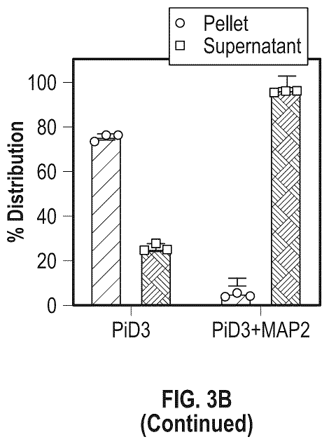

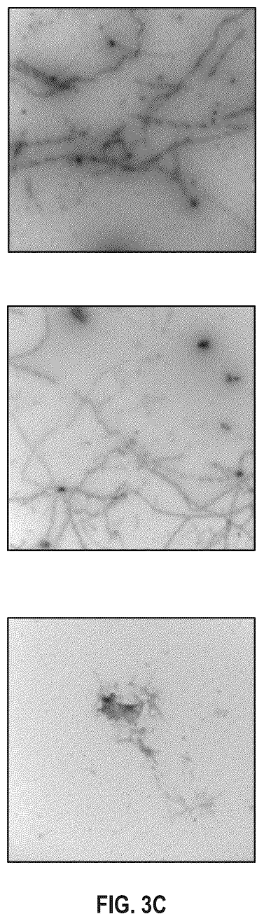

Referring to FIG. 3A, graphical representations of experimental primary Thioflavin T (ThT) fluorescence data from Real-time quaking-induced conversion (RT-QuIC) reactions of tissue extracts from Pick's disease (PiD) brains illustrate changes in behavior in the presence of MAP2. FIG. 3A contains three separate graphs arranged vertically, each corresponding to a different biological PiD sample designated as PiD 1, PiD 2, and PiD 3. Each graph plots normalized ThT fluorescence on the vertical axis against time in hours on the horizontal axis, with the time scale extending from 0 to 100 hours.

In the top graph of FIG. 3A, data for the PiD 1 sample is displayed. Gray data points represent reactions of PiD brain extracts with Tau protein alone, shown as three technical replicates labeled PiD 1-1, PiD 1-2, and PiD 1-3. Blue data points represent reactions in the presence of MAP2, labeled as PiD 1-1+MAP2, PiD 1-2+MAP2, and PiD 1-3+MAP2. The gray traces display sigmoidal growth curves characteristic of Tau aggregation, with fluorescence values rising from approximately 0.0 to between 0.6 and 1.0 over the course of the experiment. The blue traces representing reactions with MAP2 remain at baseline levels near 0.0 throughout the entire 100-hour time period, indicating inhibition of fibril formation by MAP2.

With continued reference to FIG. 3A, the middle graph shows data for the PiD 2 sample. Gray traces for PiD 2-1, PiD 2-2, and PiD 2-3 exhibit sigmoidal fluorescence curves indicative of Tau aggregation over time. Blue traces for PiD 2-1+MAP2, PiD 2-2+MAP2, and PiD 2-3+MAP2 remain at baseline fluorescence levels throughout the 100-hour measurement period. The sigmoidal growth pattern observed in the gray traces reflects the characteristic kinetics of amyloid fibril formation, with an initial lag phase followed by rapid growth and eventual plateau. The suppression of ThT fluorescence in the presence of MAP2 indicates that MAP2 prevents the formation of ThT-positive fibrillar species from PiD brain-derived seeds.

The bottom graph in FIG. 3A displays data for the PiD 3 sample. Gray traces for PiD 3-1, PiD 3-2, and PiD 3-3 show sigmoidal growth curves with fluorescence values increasing from approximately 0.0 to values between 0.6 and 1.0 over the time course. Blue traces for PiD 3-1+MAP2, PiD 3-2+MAP2, and PiD 3-3+MAP2 remain at baseline levels near 0.0 throughout the entire 100-hour time period. The consistent inhibition observed across all three biological replicates demonstrates reproducible inhibitory activity of MAP2 against Tau aggregation from PiD brain-derived seeds.

The polypeptide may be used in combination with RT-QuIC assays for amplifying and detecting Tau proteins from proteopathic seeds. RT-QuIC assays involve repeated cycles of shaking the reaction mixture followed by quiescent phases, enabling amplification of Tau proteins from minute quantities of seeds present in processed disease brain biospecimens into long fibrils. ThT dye binds to beta-sheet rich fibrillar structures and exhibits enhanced fluorescence upon binding, providing a real-time readout of fibril formation during the assay. The sigmoidal kinetic behavior observed in the gray traces reflects the nucleation-dependent polymerization mechanism of Tau fibril growth, where an initial lag phase corresponds to seed formation or activation, followed by rapid elongation and eventual plateau as monomer substrate becomes depleted.

The cofactor-free inhibition approach demonstrated in FIG. 3A preserves the native structure of disease-associated fibrils extracted from clinical patient tissue. Fibrils assembled using heparin as a cofactor have different structures from fibrils isolated from disease tissue, and the presence of heparin may cause inhibitor proteins to form amorphous aggregates that complicate interpretation of results. In the cofactor-free approach, MAP2 remains in the soluble supernatant fraction rather than forming amorphous aggregates, enabling clear assessment of inhibitory activity against disease-relevant fibril structures.

The assay conditions included incubation of brain homogenate seeds at 30 μg/ml with recombinant 3R Tau monomers at 10 μM in 10 mM sodium phosphate buffer at pH 7.4 with 10 mM dithiothreitol. Since Pick's disease is a 3R-exclusive tauopathy, 3R Tau was used as the reaction substrate. The reactions were carried out in triplicates from three different biological PiD samples, providing both technical and biological replication to assess reproducibility of the inhibitory effect. The RT-QuIC assay involved multiple cycles including a 1-minute double orbital shaking at 400 rpm followed by a 9-minute resting phase. The consistent suppression of ThT fluorescence across all nine reaction conditions containing MAP2 demonstrates robust inhibitory activity against Tau aggregation from PiD brain-derived seeds.

Referring to FIG. 3B, bar graphs show quantitative analysis of fibril formation in extracts from Pick's disease brains in the presence of MAP2. FIG. 3B presents three separate bar graph panels corresponding to three different biological samples designated PiD1, PiD2, and PiD3. Each panel displays percent distribution on the vertical axis ranging from zero to one hundred percent. Two conditions are shown for each sample: the sample alone and the sample combined with MAP2. The bars are color-coded with purple representing the pellet fraction and light blue representing the supernatant fraction.

In all three panels of FIG. 3B, the samples without MAP2 show approximately seventy-five percent of the material in the pellet fraction and approximately twenty-five percent in the supernatant fraction. The pellet fraction represents insoluble fibrillar aggregates that sediment during ultracentrifugation, while the supernatant fraction represents soluble monomeric protein that remains in solution. The distribution of approximately seventy-five percent in the pellet fraction indicates that Tau monomers have been recruited onto the PiD brain-derived seeds and converted into insoluble fibrillar species during the RT-QuIC reaction.

With continued reference to FIG. 3B, when MAP2 is present, the distribution shifts dramatically across all three biological samples. The pellet fraction decreases to less than ten percent, while the supernatant fraction increases to approximately ninety to one hundred percent. The shift from approximately seventy-five percent pellet to greater than ninety percent supernatant when MAP2 is present demonstrates that the polypeptide reduces Tau aggregation by at least 90% as measured by sedimentation assay quantification. Error bars are visible on each bar indicating standard deviation from triplicate measurements, demonstrating reproducibility of the quantitative assessment across technical replicates.

The polypeptide inhibition may be assessed using ultracentrifugation-based sedimentation assays at 260,000 g for 30 minutes. The technical replicates were combined and subjected to ultracentrifugation at 260,000 g for 30 minutes to separate insoluble fibrillar aggregates in the pellet from soluble monomeric protein in the supernatant. The supernatant and pellet fractions were analyzed by 12% sodium dodecyl sulfate-polyacrylamide gel electrophoresis (SDS-PAGE) followed by Coomassie staining. Quantification of pellet and supernatant fractions in the gel was performed by densitometric analysis using image analysis software.

The ultracentrifugation-based sedimentation assay provides a direct measurement of the physical state of Tau protein, distinguishing between soluble monomeric species and insoluble fibrillar aggregates based on sedimentation behavior under high centrifugal force. The quantitative nature of densitometric analysis enables precise determination of the percent distribution between pellet and supernatant fractions, allowing calculation of inhibition efficiency. The sedimentation assay complements the ThT fluorescence data by providing an independent measure of fibril formation that does not rely on dye binding properties.

The consistent shift to greater than ninety percent supernatant across all three biological PiD samples when MAP2 is present indicates that MAP2 maintains Tau protein in the soluble monomeric state by preventing recruitment onto fibril seeds. The retention of Tau in the supernatant fraction demonstrates that MAP2 interferes with the fibril elongation process rather than causing precipitation or non-specific aggregation of Tau protein.

Referring to FIG. 3C, three electron microscopy images arranged vertically show fibril formation by Tau 3R in the presence of tissue extracts from brains of Pick's disease patients. Each image corresponds to a different biological PiD sample, with the top image showing results from a first PiD patient sample, the middle image showing results from a second PiD patient sample, and the bottom image showing results from a third PiD patient sample. The images display grayscale fields containing dark elongated fibrillar structures and scattered dark spots against a lighter background, representing the morphology of Tau fibrils formed during the RT-QuIC reactions.

With continued reference to FIG. 3C, the dark elongated fibrillar structures visible in each image exhibit characteristic morphology of amyloid fibrils, including extended linear profiles with varying lengths. The fibrillar structures appear as electron-dense features against the lighter background of the carbon-coated copper grid support. The scattered dark spots visible in the images may represent smaller aggregates, fibril fragments, or staining artifacts. The images include scale bars and technical information in the lower portions indicating the microscopy parameters used during image acquisition.

The polypeptide inhibition may be validated using electron microscopy imaging to confirm absence of fibrillar species. Electron microscopy provides direct visualization of fibril morphology and enables assessment of whether fibrillar structures are present or absent in reaction products. The negative stain electron microscopy technique involves applying reaction end products to Formvar/carbon-coated copper grids with 200 mesh, followed by incubation for 90 seconds, removal of excess liquid, and exposure to 2% uranyl acetate filtered through a 0.2 μm syringe for another 90 seconds. After blotting with filter paper, the grids may be left to air dry for 5 minutes before imaging. Transmission electron microscopy may be performed using instruments operating at 100 kV, with images captured on CCD cameras for subsequent analysis.

Electron microscopy validation enables assessment of fibril morphology independent of dye binding properties or sedimentation behavior. The morphological information obtained from electron microscopy images may also reveal characteristics of fibril structure including length distribution, branching patterns, and association into higher-order assemblies.

The electron microscopy images in FIG. 3C complement the ThT fluorescence data and sedimentation assay results by providing visual confirmation of fibril formation from PiD brain-derived seeds. The combination of ThT fluorescence kinetics, sedimentation analysis, and electron microscopy imaging provides multiple independent lines of evidence for assessing polypeptide inhibitory activity against Tau aggregation from disease brain-derived seeds.

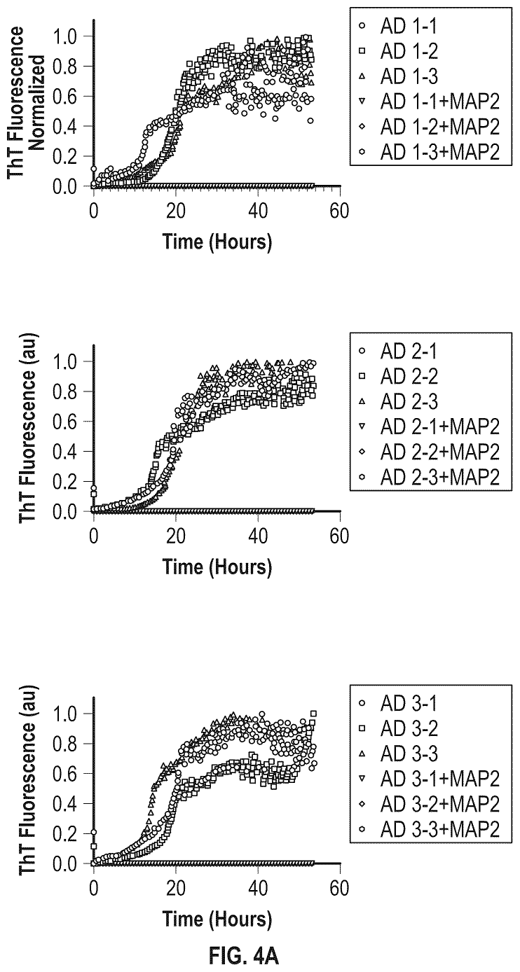

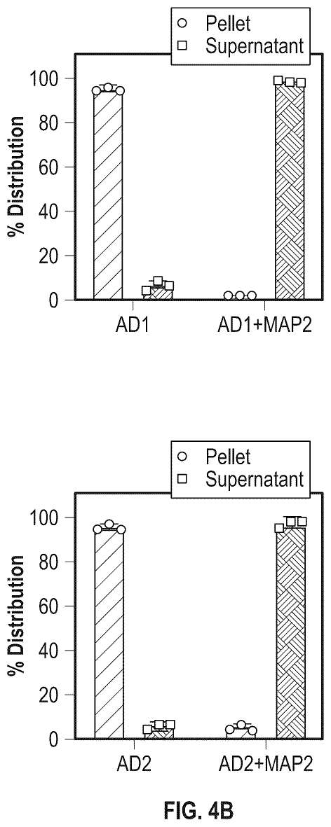

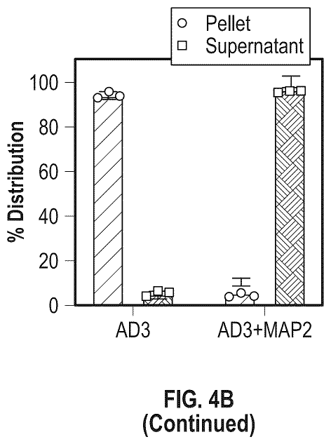

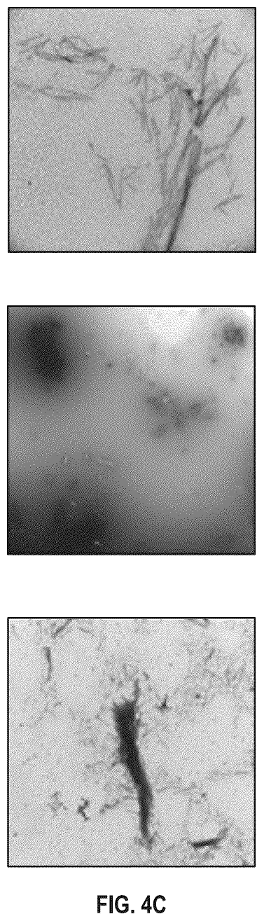

Referring to FIG. 4A, graphical representations of experimental primary Thioflavin T (ThT) fluorescence data from Real-time quaking-induced conversion (RT-QuIC) reactions of tissue extracts from Alzheimer's disease (AD) brains illustrate changes in behavior in the presence of MAP2. FIG. 4A contains three separate graphs arranged vertically, each corresponding to a different biological AD sample designated as AD 1, AD 2, and AD 3. Each graph plots ThT Fluorescence in arbitrary units on the vertical axis ranging from 0.0 to 1.0, and Time in Hours on the horizontal axis ranging from 0 to 60.

Gray traces represent reactions of AD brain extracts with Tau protein alone, shown as three technical replicates for each biological sample. Orange traces represent reactions in the presence of MAP2. Across all three biological samples, the gray traces exhibit sigmoidal growth curves characteristic of Tau aggregation, while the orange traces remain at or near baseline fluorescence levels throughout the entire 100-hour time period, indicating inhibition of fibril formation by MAP2. The consistent inhibition observed across all three biological replicates demonstrates reproducible inhibitory activity of MAP2 against Tau aggregation from AD brain-derived seeds.

The polypeptide may be used with brain tissue extracts processed by homogenization, centrifugation, and sonication. Frozen brain tissue samples from the frontal cortex of AD post-mortem patients may be homogenized in buffer containing 10 mM HEPES at pH 7.4, 5 mM EDTA, 150 mM NaCl, and 0.1% Triton X-100 with protease inhibitor. The homogenate may be subjected to centrifugation for 10 minutes at 3000 g, followed by collection of the supernatant fraction and sonication for 2 minutes to disperse aggregates. Total protein concentration in sonicated samples may be determined by bicinchoninic acid (BCA) assay, followed by aliquoting, flash-freezing, and storage at −80° C. for subsequent use in RT-QuIC assays.

The suppression of ThT fluorescence in the presence of MAP2 indicates that MAP2 prevents the formation of ThT-positive fibrillar species from AD brain-derived seeds. Since Alzheimer's disease is a mixed 3R+4R tauopathy, both 3R Tau and 4R Tau may be used as reaction substrates with AD brain-derived seeds. The assay conditions included incubation of brain homogenate seeds at 30 μg/ml with recombinant Tau monomers at 10 μM in 10 mM sodium phosphate buffer at pH 7.4 with 10 mM dithiothreitol, in the absence of cofactors such as heparin or dextran sulfate, thereby preserving the native structure of disease-associated fibrils extracted from clinical patient tissue.

Referring to FIG. 4B, bar graphs show quantitative analysis of fibril formation using ultracentrifugation-based sedimentation assays of RT-QuIC reactions for tissue extracts from Alzheimer's disease brains. FIG. 4B presents three separate bar graph panels corresponding to three different biological AD samples designated AD1, AD2, and AD3. Each panel displays percent distribution on the vertical axis ranging from zero to one hundred percent. Two conditions are shown for each sample: the AD sample alone and the AD sample combined with MAP2. Each condition includes two bars representing the pellet fraction shown in purple and the supernatant fraction shown in light blue.

For all three AD samples tested alone in FIG. 4B, the pellet fraction approaches approximately ninety to one hundred percent while the supernatant fraction remains minimal at approximately zero to ten percent. The high pellet fraction indicates that Tau monomers have been recruited onto the AD brain-derived seeds and converted into insoluble fibrillar species during the RT-QuIC reaction, correlating with the sigmoidal ThT fluorescence curves observed in FIG. 4A.

With continued reference to FIG. 4B, when MAP2 is present with each AD sample, the distribution reverses such that the supernatant fraction approaches approximately ninety to one hundred percent while the pellet fraction decreases to approximately zero to ten percent. The shift from approximately ninety to one hundred percent pellet to approximately ninety to one hundred percent supernatant when MAP2 is present indicates that the polypeptide reduces Tau aggregation by at least 90% as measured by sedimentation assay quantification. Error bars are visible on each bar indicating standard deviation from triplicate measurements.

The polypeptide remains in the supernatant fraction without forming amorphous aggregates during inhibition assays. In prior techniques using cofactors such as heparin, inhibitor proteins appeared in the pellet fraction and formed amorphous aggregates, complicating interpretation of results. In the cofactor-free approach demonstrated in FIG. 4B, MAP2 remains in the soluble supernatant fraction along with the Tau protein, indicating that MAP2 does not self-aggregate or form non-specific precipitates during the inhibition assay.

The consistent reversal of protein distribution across all three biological AD samples when MAP2 is present indicates reproducible inhibitory activity against Tau aggregation from AD brain-derived seeds obtained from multiple patients. The sedimentation assay results complement the ThT fluorescence data by providing an independent measure of fibril formation that does not rely on dye binding properties. The retention of Tau in the supernatant fraction demonstrates that MAP2 maintains Tau protein in the soluble monomeric state by preventing recruitment onto fibril seeds, consistent with a capping mechanism whereby MAP2 engages with fibril ends and blocks access for incoming Tau monomers.

Referring to FIG. 4C, three electron microscopy images arranged vertically show fibril formation by Tau 3R in the presence of tissue extracts from brains of Alzheimer's disease patients. Each image corresponds to a different biological AD sample, providing visual confirmation of fibril formation from AD brain-derived seeds across multiple patient sources. The images display grayscale fields captured using transmission electron microscopy with negative staining to enhance contrast of protein structures against the carbon-coated copper grid support.

The top image in FIG. 4C displays elongated fibrillar structures with branching patterns against a grainy background. The fibrillar structures exhibit characteristic morphology of Tau fibrils, including extended linear profiles with varying lengths and branching points where fibrils appear to diverge or associate. The branching patterns visible in the top image may represent fibril fragmentation, secondary nucleation events, or lateral association of multiple fibril strands. The presence of elongated fibrillar structures in the top image correlates with the sigmoidal ThT fluorescence curves and high pellet fractions observed in reactions without MAP2 for the corresponding AD sample.

With continued reference to FIG. 4C, the middle image shows a darker field with diffuse staining and less distinct fibrillar morphology compared to the top image. The middle image represents a different sample or condition from the same experimental set, with the variation in image appearance reflecting differences in sample preparation, staining intensity, or fibril density across the biological replicates. The diffuse staining pattern visible in the middle image may indicate the presence of smaller aggregates, fibril fragments, or amorphous protein deposits in addition to or instead of extended fibrillar structures.

The bottom image in FIG. 4C presents a clearer view of a single elongated fibrillar structure against a lighter background with surrounding granular material. The elongated fibrillar structure visible in the bottom image demonstrates the characteristic morphology of Tau fibrils amplified from Alzheimer's disease brain extracts, with an extended linear profile spanning a substantial portion of the field of view. The surrounding granular material may represent smaller aggregates, monomeric protein, or staining artifacts. Each image includes scale bar information and imaging parameters displayed at the bottom of the respective panels, enabling assessment of fibril dimensions and comparison across samples.

The polypeptide inhibition may be validated using electron microscopy imaging, as described above for FIG. 3C, to confirm presence or absence of fibrillar species in reaction products. The negative stain electron microscopy technique involves applying reaction end products to Formvar/carbon-coated copper grids with 200 mesh, followed by incubation for 90 seconds, removal of excess liquid, and exposure to 2% uranyl acetate filtered through a 0.2 μm syringe for another 90 seconds. After blotting with filter paper, the grids may be left to air dry for 5 minutes before imaging.

The morphological information obtained from electron microscopy images reveals characteristics of fibril structure including length distribution, branching patterns, and association into higher-order assemblies that may not be apparent from ThT fluorescence or sedimentation data alone. The branching patterns visible in the top image of FIG. 4C demonstrate the complex morphology that Tau fibrils may exhibit when amplified from disease brain-derived seeds, providing structural context for understanding the fibril elongation process that polypeptide constructs may inhibit.

As further shown in FIG. 4C, the electron microscopy images complement the ThT fluorescence data and sedimentation assay results by providing visual confirmation of fibril formation from AD brain-derived seeds.

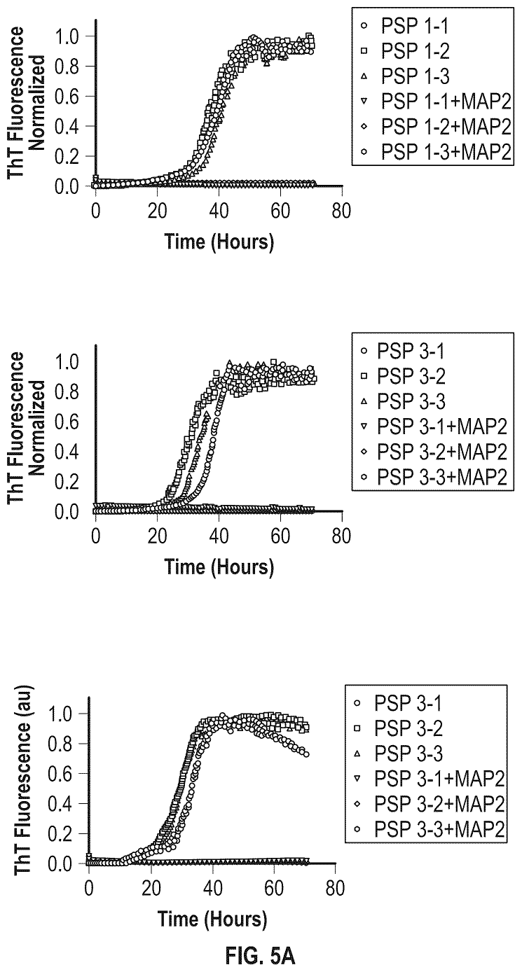

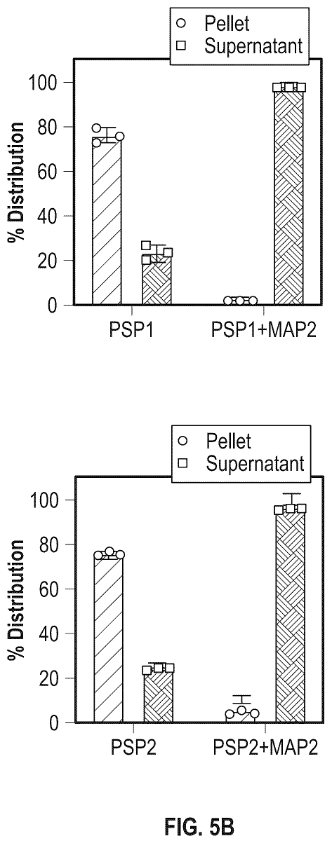

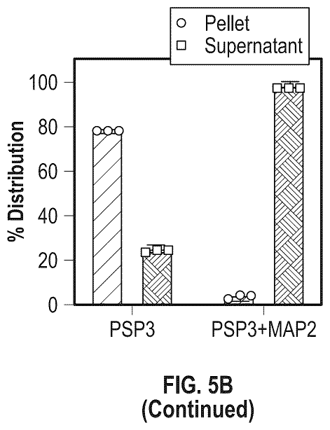

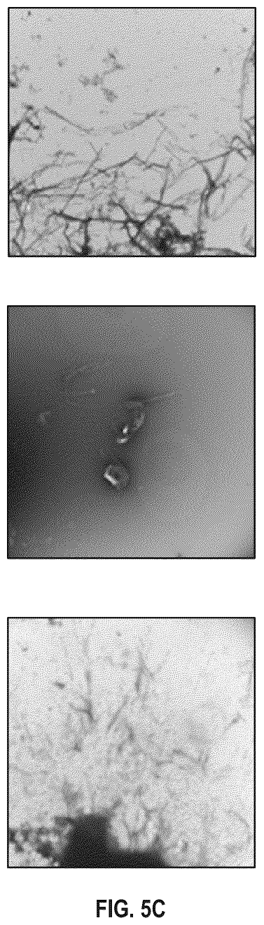

Referring to FIG. 5A, graphical representations of experimental primary Thioflavin T (ThT) fluorescence data from Real-time quaking-induced conversion (RT-QuIC) reactions of tissue extracts from progressive supranuclear palsy (PSP) brains illustrate changes in behavior in the presence of MAP2. FIG. 5A contains three separate graphs arranged vertically, each corresponding to a different biological PSP sample designated as PSP 1, PSP 2, and PSP 3. Each graph plots ThT Fluorescence Normalized on the vertical axis ranging from 0.0 to 1.0, and Time in Hours on the horizontal axis ranging from 0 to 80.

Gray traces represent reactions of PSP brain extracts with Tau protein alone, while green traces represent reactions in the presence of MAP2. Across all three biological samples, the gray traces exhibit sigmoidal growth curves with the growth phase occurring between approximately 10 and 40 hours, followed by a plateau phase. The green traces remain at or near baseline fluorescence levels throughout the entire 80-hour time period, indicating inhibition of fibril formation by MAP2. The consistent inhibition observed across all three biological replicates demonstrates reproducible inhibitory activity of MAP2 against Tau aggregation from PSP brain-derived seeds.

The polypeptide inhibition assays may be performed at 37° C. in 10 mM sodium phosphate buffer at pH 7.4 with 10 mM dithiothreitol (DTT). Since progressive supranuclear palsy is a 4R-exclusive tauopathy, 4R Tau may be used as the reaction substrate with PSP brain-derived seeds. The reactions were carried out in triplicates from three different biological PSP samples, providing both technical and biological replication to assess reproducibility of the inhibitory effect across multiple patient-derived seed sources. The cofactor-free inhibition approach preserves the native structure of disease-associated fibrils extracted from clinical patient tissue, enabling assessment of inhibitory activity against PSP-specific Tau fibril conformations.

The broad-spectrum activity of MAP2 against PSP fibrils, combined with the inhibitory activity against PiD and AD fibrils demonstrated in previous figures, indicates that a single MAP2-based construct may inhibit Tau aggregation across multiple tauopathies characterized by different fibril conformations. In some cases, the broad-spectrum capping behavior may enable development of therapeutic agents effective against multiple tauopathies without requiring disease-specific modifications to the polypeptide construct.

Referring to FIG. 5B, bar graphs show quantitative analysis of fibril formation using sedimentation assays of RT-QuIC reactions for PSP brain samples, performed as described above for FIG. 3B. FIG. 5B presents three separate graphs corresponding to three different biological PSP samples designated PSP1, PSP2, and PSP3. Each graph displays percent distribution on the vertical axis ranging from 0 to 100. The horizontal axis of each graph shows two conditions: PSP alone and PSP combined with MAP2. Two categories are represented in each graph, indicated by purple bars for pellet fraction and light blue bars for supernatant fraction.

In the PSP alone condition across all three samples shown in FIG. 5B, the pellet fraction shows approximately 75 to 80 percent distribution while the supernatant fraction shows approximately 20 to 25 percent distribution, indicating that Tau monomers have been recruited onto the PSP brain-derived seeds and converted into insoluble fibrillar species during the RT-QuIC reaction.

With continued reference to FIG. 5B, in the PSP combined with MAP2 condition across all three samples, the pellet fraction is reduced to near zero percent while the supernatant fraction increases to approximately 95 to 100 percent distribution. The shift from approximately 75 to 80 percent pellet to near zero percent pellet when MAP2 is present demonstrates that the polypeptide inhibits Tau aggregation from PSP brain-derived seeds. Error bars are visible on the data points indicating standard deviation from triplicate measurements.

The polypeptide reduces Tau aggregation by greater than 90% as measured by sedimentation assay quantification for PSP brain-derived seeds. The quantitative shift from predominantly pellet to predominantly supernatant when MAP2 is present correlates with the suppressed ThT fluorescence observed in FIG. 5A, providing independent confirmation of inhibitory activity. The consistent shift to greater than 95 percent supernatant across all three biological PSP samples indicates reproducible inhibitory activity against Tau aggregation from PSP brain-derived seeds obtained from multiple patients. The polypeptide remains in the soluble supernatant fraction without forming amorphous aggregates during inhibition assays, demonstrating that MAP2 interferes with the fibril elongation process through specific capping interactions rather than causing precipitation or non-specific aggregation of proteins.

Referring to FIG. 5C, electron microscopy images show fibril formation by Tau 3R in the presence of tissue extracts from brains of PSP patients. FIG. 5C presents three separate grayscale electron microscopy images arranged vertically, each corresponding to a different PSP brain sample. The images were captured using transmission electron microscopy with negative staining to enhance contrast of protein structures against the carbon-coated copper grid support.

The top image in FIG. 5C shows a field of view with scattered dark linear structures and branching filamentous material against a light gray background. The dark linear structures exhibit characteristic morphology of amyloid fibrils, including extended profiles with varying lengths. The branching filamentous material visible in the top image may represent fibril fragmentation events, secondary nucleation, or lateral association of multiple fibril strands. Scale information and imaging parameters are displayed at the bottom of the image, enabling assessment of fibril dimensions.

With continued reference to FIG. 5C, the middle image displays a cleaner field with fewer visible structures compared to the top image. The middle image shows what appears to be two small dark aggregates or particles with some faint linear features extending from the aggregates. The variation in image appearance across the three biological samples reflects differences in fibril density, sample preparation, or staining intensity across the PSP patient-derived seed sources. The presence of aggregates with extending linear features may represent early-stage fibril growth or fibril nucleation events captured during the RT-QuIC reaction.

The bottom image in FIG. 5C presents a more complex field containing numerous dark spots, granular material, and interconnected fibrillar structures distributed across the viewing area. The interconnected fibrillar structures visible in the bottom image demonstrate the network-like morphology that Tau fibrils may exhibit when amplified from PSP brain-derived seeds. The numerous dark spots and granular material distributed throughout the field may represent smaller aggregates, fibril fragments, monomeric protein deposits, or staining artifacts. Each image includes technical information regarding the electron microscopy acquisition parameters positioned at the lower portion of the respective panels.

The polypeptide inhibition may be validated using electron microscopy imaging, as described above for FIG. 3C, to confirm presence or absence of fibrillar species in reaction products from PSP brain-derived seeds.

The morphological information obtained from electron microscopy images reveals characteristics of fibril structure including length distribution, branching patterns, network formation, and association into higher-order assemblies. In reactions containing MAP2, the absence of fibrillar species in electron microscopy images would correlate with the suppressed ThT fluorescence shown in FIG. 5A and the shift to supernatant fractions shown in FIG. 5B.

As further shown in FIG. 5C, the electron microscopy images complement the ThT fluorescence data and sedimentation assay results by providing visual confirmation of fibril formation from PSP brain-derived seeds.

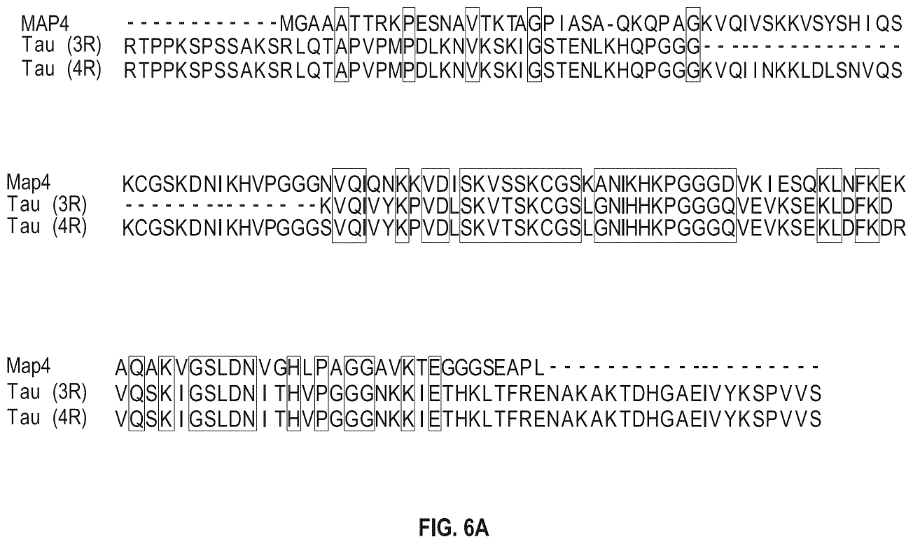

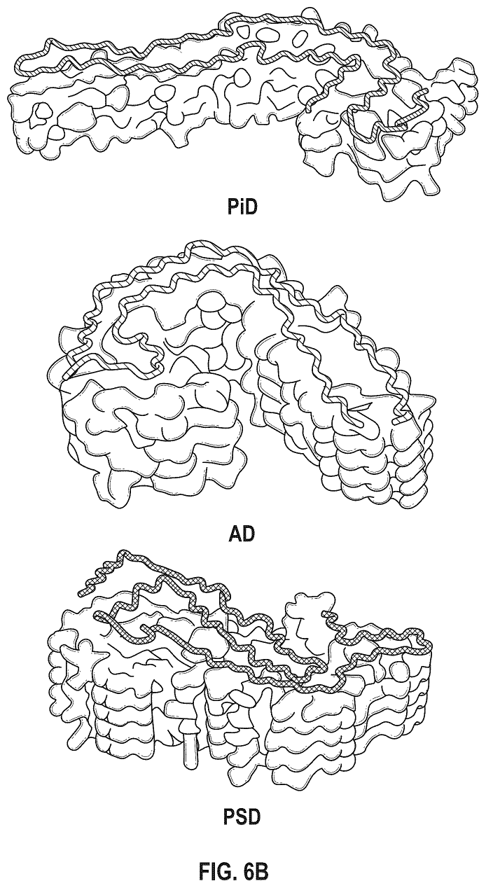

Referring to FIG. 6A, a sequence alignment graphically illustrates regions of similarity between MAP4 and 3R/4R Tau proteins. The alignment shows three protein sequences arranged vertically for comparison: MAP4 at the top, Tau (3R) in the middle, and Tau (4R) at the bottom. Each sequence is displayed as a string of single-letter amino acid codes, enabling direct comparison of residue identity and conservation across the three proteins.

Shaded regions within the sequences shown in FIG. 6A indicate high conservation of amino acids between MAP4 and the Tau variants. The shaded regions correspond to positions where MAP4 shares identical or similar residues with the 3R and 4R Tau isoforms, demonstrating the sequence homology that enables MAP4 to engage with Tau fibril surfaces through complementary interactions. The conserved regions correspond to microtubule binding repeat domains that form the structured core of pathological Tau filaments in various tauopathies. Sequence alignment analysis reveals that MAP2 (residues R305-E410 of UniProt P11137-2) shares 80.3% and 48.9% sequence similarity with the 3R and 4R Tau repeat regions respectively, while MAP4 (residues A962-L1097 of UniProt P27816-1) shares 62.3% and 67.1% sequence similarity with the 3R and 4R Tau repeat regions respectively.

With continued reference to FIG. 6A, dashes in the Tau (3R) sequence represent gaps introduced to optimize the alignment between the proteins. The gap region in the 3R sequence reflects the absence of the second repeat region characteristic of the 3R isoform compared to the 4R isoform. The 3R Tau isoform contains three microtubule binding repeats, while the 4R Tau isoform contains four microtubule binding repeats, with the additional repeat in 4R Tau corresponding to the gap region visible in the 3R sequence alignment. The alignment demonstrates that MAP4 shares sequence similarity with both the 3R and 4R isoforms of Tau protein, particularly in regions that form the structured core of pathological Tau filaments.

The sequences of MAP4 and Tau may be retrieved from UniProt, and alignments may be performed using Clustal Omega or similar alignment tools. The polypeptide constructs may use specific regions of MAP4 corresponding to residues A962-L1097 of UniProt P27816-1. The repeat region of MAP4 encompassing residues A962-L1097 contributes to the high sequence similarity with 3R or 4R Tau and encompasses the structured core that engages with disease-associated fibril ends.

As further shown in FIG. 6A, MAP4 possesses sequence motifs corresponding to aggregation-prone regions in Tau protein. MAP4 contains a motif corresponding to the 275VQIINK280 region in Tau, with the sequence VQIVSK at the corresponding position. MAP4 also contains a motif corresponding to the 306VQIVYK311 region in Tau, with the sequence VQIQNK at the corresponding position. The natural substitutions in MAP4 at these positions may allow MAP4 to bind to Tau fibril surfaces while preventing self-aggregation or cross-aggregation with Tau fibrils.

As further shown in FIG. 6A, MAP4 possesses sequence motifs corresponding to aggregation-prone regions in Tau protein. The 275VQIINK280 and 306VQIVYK311 motifs in Tau have been shown to be critical for Tau assembly into disease-associated fibrils, serving as key drivers of the fibrillation process. MAP4 contains natural substitutions at these positions: the sequence VQIVSK at the position corresponding to the 275VQIINK280 region in Tau, and the sequence VQIQNK at the position corresponding to the 306VQIVYK311 region in Tau. Substitutions at these aggregation-prone positions may affect the fibrillation properties of the motifs. For example, residues forming attractive inter-molecular contacts between Tau layers are conserved in MAP proteins, which may stabilize binding to fibril surfaces, while nonconserved regions may not stack in the original arrangement, potentially changing fibril morphology and preventing recruitment of further Tau monomers on the altered surfaces. These natural substitutions in MAP4 may allow MAP4 to bind to Tau fibril surfaces while preventing self-aggregation or cross-aggregation with Tau fibrils, providing a molecular basis for the capping behavior observed in experimental assays. Similarly, MAP2 lacks the 275VQIINK280 motif due to the absence of the second repeat region, and possesses the sequence 306VQIVTK311 at the position corresponding to Tau's VQIVYK motif. The natural sequence variations at these aggregation-prone positions in both MAP2 and MAP4 may enable these proteins to engage with Tau fibril ends through complementary interactions while lacking the self-assembly propensity that would otherwise lead to fibril propagation.

The availability of MAP4 as an additional capper molecule provides a framework for comparative analysis of structure-activity relationships. MAP4 and MAP2 share sequence homology with Tau but differ in specific residue positions, and these differences may confer different binding properties or selectivity for particular Tau fibril conformations, enabling rational design of constructs with tailored activity profiles.

Referring to FIG. 6B, homology-based modeling of MAP4 into different types of disease-associated Tau folds is illustrated. FIG. 6B shows three-dimensional structural representations of MAP4 chains modeled into Tau fibril conformations associated with three distinct tauopathies: Pick's disease (PiD), Alzheimer's disease (AD), and progressive supranuclear palsy (PSP). In each representation, a MAP4 chain is positioned along the surface of a gray Tau fibril structure, demonstrating that MAP4 may adopt conformations complementary to the respective disease-specific fibril architectures.

The top structure in FIG. 6B depicts the Pick's disease fold, where a MAP4 chain shown in blue traces along the surface of the gray Tau fibril structure. The blue MAP4 chain follows the characteristic curved conformation of the Pick's disease-associated Tau fibril, with the modeled MAP4 adopting the meandering path that defines the 3R Tau fibril core structure found in PiD. The three-dimensional alignment demonstrates that MAP4 may acquire the PiD-specific Tau fold through conformational adaptation enabled by the sequence similarity between MAP4 and the Tau core region.

With continued reference to FIG. 6B, the middle structure shows the Alzheimer's disease fold, with a MAP4 chain displayed in orange following the characteristic curved conformation of the AD-associated Tau fibril rendered in gray. The orange MAP4 chain adopts the structural topology characteristic of the Alzheimer's disease Tau fibril core, demonstrating that MAP4 may fold into the AD-specific conformation. The gray surface in the middle representation depicts the underlying Tau fibril architecture while the orange chain indicates the modeled MAP4 protein positioned along the fibril surface in a manner complementary to the AD fold.

The bottom structure in FIG. 6B presents the progressive supranuclear palsy fold, where a MAP4 chain appears in green positioned along the more elongated gray Tau fibril structure. The green MAP4 chain conforms to the structural architecture associated with PSP Tau fibrils, which exhibit a more extended conformation compared to the PiD and AD folds. The three-dimensional alignment of the modeled MAP4 chain over the PSP Tau fibril demonstrates that MAP4 may acquire the PSP-specific fold, enabling engagement with fibril surfaces from this 4R-exclusive tauopathy.

The polypeptide may be validated using homology-based modeling with SwissModel workspace. SwissModel workspace provides automated protein structure homology modeling capabilities that enable prediction of three-dimensional protein structures based on sequence alignment with template structures of known conformation. The cryo-EM structures of disease-associated Tau fibrils from PiD, AD, and PSP serve as templates for modeling MAP4 into the respective disease-specific folds. The modeling process involves alignment of the MAP4 sequence with the Tau fibril template sequence, followed by construction of a three-dimensional model based on the template structure and the aligned sequence.

As further shown in FIG. 6B, three-dimensional alignments were generated using PyMol molecular graphics software. PyMol enables visualization and structural superposition of modeled MAP4 chains over Tau fibril structures, allowing assessment of how well the modeled MAP4 conformations match the underlying disease-associated Tau folds. The structural superposition shown in FIG. 6B demonstrates that MAP4 chains modeled into PiD, AD, and PSP folds align with the respective Tau fibril surfaces without steric clashes, indicating favorable structural complementarity between MAP4 and the disease-associated fibril architectures.

Homology-based modeling enables assessment of whether a polypeptide sequence may adopt a particular disease-associated fold prior to experimental validation, informing selection of constructs for further development. The three-dimensional structural representations generated through homology modeling provide visualization of the predicted binding mode and contact surface between the polypeptide and the fibril structure.

The conformational adaptability of MAP4 demonstrated in FIG. 6B indicates that MAP4-based constructs may engage with fibril surfaces from multiple tauopathies through conformational adaptation, similar to MAP2.

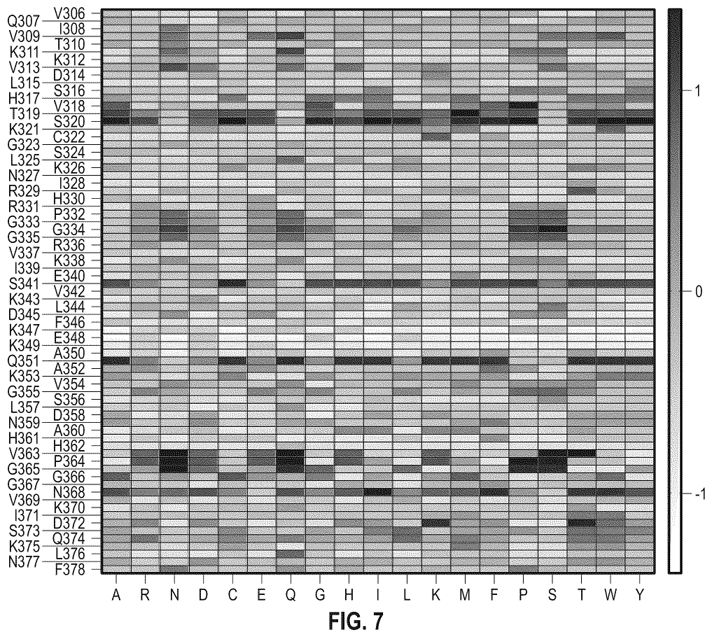

Referring to FIG. 7, a saturation mutagenesis map of MAP2 in a bound state with a Tau fibril having an Alzheimer's disease fold is presented. FIG. 7 displays a heatmap with amino acid positions of MAP2 listed along the vertical axis, ranging from V306 at the top to F378 at the bottom. The horizontal axis displays single-letter amino acid codes representing possible substitutions, including A, R, N, D, C, E, Q, G, H, I, L, K, M, F, P, S, T, W, and Y. Each cell in the heatmap represents a mutation-induced change in the binding energy of MAP2 with Tau, enabling systematic assessment of how each possible amino acid substitution at each position affects the interaction between MAP2 and the Tau fibril surface.

With continued reference to FIG. 7, a color scale bar is provided on the right side of the figure, ranging from approximately negative one to positive one. Blue coloring indicates stabilizing mutations that enhance binding affinity between MAP2 and the Tau fibril, while red coloring indicates destabilizing mutations that reduce binding affinity. White or neutral coloring represents mutations with minimal effect on binding energy. The heatmap reveals patterns of mutation tolerance across different positions, with certain residue positions showing predominantly blue cells indicating favorable substitutions that may enhance capping activity, while other positions display red cells indicating unfavorable substitutions that may reduce engagement with the fibril surface.

The saturation mutagenesis map shown in FIG. 7 was created using a FoldX mutagenesis engine followed by calculation of binding energies with a Prodigy tool. The FoldX mutagenesis engine enables systematic in silico substitution of each amino acid at each position in the MAP2 sequence, generating structural models of each mutant variant in the context of the MAP2-Tau fibril complex. The Prodigy tool calculates binding energies for each mutant complex based on protein-protein contacts, enabling quantitative assessment of how each substitution affects the interaction strength between MAP2 and the Tau fibril surface.

The polypeptide may be validated using saturation mutagenesis mapping to identify stabilizing and destabilizing mutations. The systematic nature of saturation mutagenesis enables comprehensive assessment of the mutational landscape, identifying positions that tolerate substitution and positions where specific amino acids enhance or reduce binding affinity. Stabilizing mutations identified through the saturation mutagenesis map may be incorporated into polypeptide constructs to enhance binding affinity for Tau fibril surfaces. Destabilizing mutations identified through the map may be avoided or may be selectively incorporated to reduce binding to particular fibril conformations when selectivity is desired.

As further shown in FIG. 7, the saturation mutagenesis approach may be applied to other MAP2-Tau fibril complexes from different tauopathies such as Pick's disease and progressive supranuclear palsy for design of disease-specific MAP2 cappers. The polypeptide may be mutagenized to maximize complementarity with specific fibril conformers found in different tauopathies. Since Tau fibrils from different tauopathies exhibit distinct structural conformations with varying cross-sectional packing interactions, the optimal mutations for enhancing binding affinity may differ across disease-associated fibril types. Saturation mutagenesis mapping performed on MAP2 complexed with PiD or PSP fibril structures may reveal different patterns of stabilizing and destabilizing mutations compared to the AD fold shown in FIG. 7.

Saturation mutagenesis mapping enables identification of mutations that may not be predicted through sequence analysis or structural inspection alone. The quantitative binding energy values generated through the FoldX and Prodigy workflow enable ranking of mutations by their predicted effect on binding affinity, informing prioritization of variants for experimental validation. Combinations of multiple stabilizing mutations may be incorporated into a single polypeptide construct to achieve additive or synergistic enhancement of binding affinity.

The saturation mutagenesis approach also enables development of disease-specific cappers with selective activity against particular Tau fibril conformations. Mutations that stabilize binding to one fibril conformation while destabilizing binding to another conformation may be identified by comparing saturation mutagenesis maps generated for different disease-associated fibril structures.

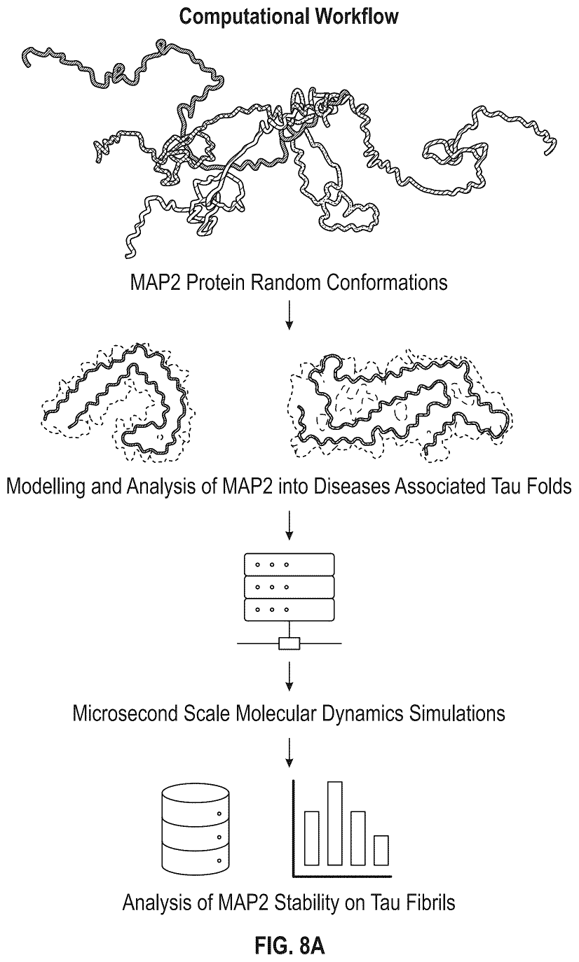

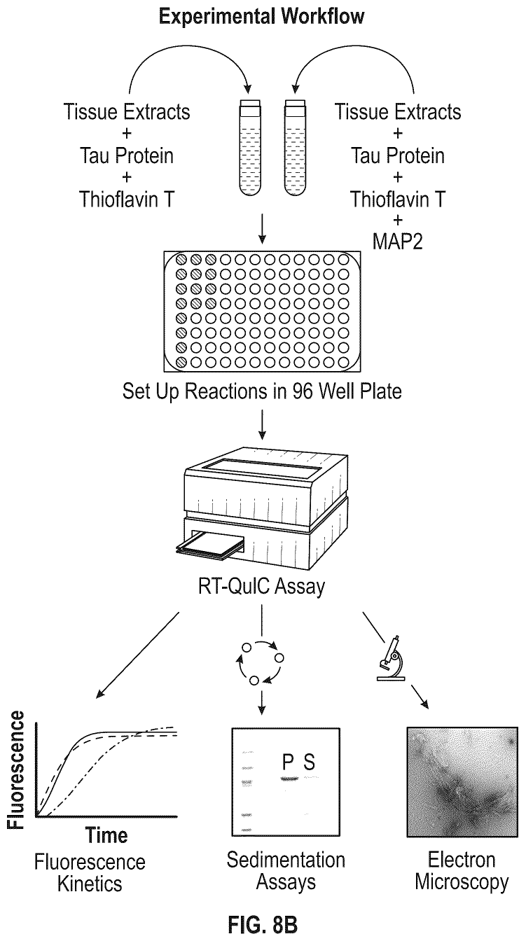

Referring to FIG. 8A, an exemplary computational workflow suitable for analyzing the inhibition of Tau fibril aggregation by modified proteins is illustrated. FIG. 8A presents a sequential process flow that enables systematic assessment of polypeptide binding potential and stability on disease-associated Tau fibril surfaces through computational modeling and simulation approaches.

The workflow shown in FIG. 8A begins with MAP2 protein random conformations, depicted as disordered protein structures shown in multiple colors representing different conformational states. The disordered nature of MAP2 in solution reflects the intrinsically disordered character of the microtubule binding repeat region prior to engagement with a structured template. The multiple conformational states represented in the initial stage indicate the conformational ensemble that MAP2 may sample in the absence of a binding partner.

With continued reference to FIG. 8A, the process proceeds to a modeling and analysis step where MAP2 is modeled into disease-associated Tau folds. The modeling step is represented by protein structure illustrations showing the folded conformations that MAP2 may adopt when interacting with different disease-specific Tau fibril structures. During the modeling step, the disordered MAP2 protein is homology modeled to assess folding behavior into different disease-associated Tau folds such as those found in Pick's disease, Alzheimer's disease, and progressive supranuclear palsy. The homology modeling approach uses cryo-EM structures of disease-associated Tau fibrils as templates for predicting the three-dimensional conformation that MAP2 may adopt when engaged with each fibril type.

Following the modeling step shown in FIG. 8A, the workflow advances to microsecond scale molecular dynamics simulations, represented by a server or computing infrastructure icon. The computational step involves subjecting the MAP2-Tau fibril complexes to long-duration atomistic simulations to assess the stability of the interactions over extended timescales. The microsecond scale simulations enable assessment of whether MAP2 remains stably associated with the Tau fibril surface or dissociates over time, providing information about the durability of the capping interaction.

The molecular dynamics simulations may be performed using GROMACS software or equivalent molecular dynamics packages. GROMACS provides capabilities for atomistic simulation of biomolecular systems using force field parameters that describe interatomic interactions. The simulations may employ force fields such as Charmm27 and water models such as SPC/E to solvate the system. The simulation protocol may include energy minimization using steepest descent algorithms, followed by equilibration phases including NVT equilibration at 310 K using V-rescale thermostat and NPT equilibration at 1 bar using Parrinello-Rahman barostat. Production molecular dynamics runs may be performed for microsecond timescales with femtosecond time steps, with covalent bonds constrained using LINCS algorithm and long-range electrostatics calculated using Particle Mesh Ewald method.

As further shown in FIG. 8A, the final step in the workflow involves analysis of MAP2 stability on Tau fibrils, depicted by a database icon alongside a bar graph. The analysis step evaluates the binding affinity and stability of MAP2 on various disease-associated Tau fibrils through energy calculations and structural assessments. The efficiency of MAP2 to cap different fibrils may be evaluated by binding affinity and energy analyses of MAP2-Tau and Tau-Tau layers in the docked complexes.

The binding affinity analyses may be performed using protein-protein contacts-based approaches such as the Prodigy tool. The Prodigy tool calculates binding energies based on intermolecular contacts between protein chains, enabling quantitative comparison of MAP2-Tau interaction strengths with native Tau-Tau layer interactions within the fibril structure. The binding energy analyses may be averaged over portions of the simulation trajectory, such as the last 100 nanoseconds, to obtain representative values that account for conformational fluctuations during the simulation.

The stability behavior of MAP2 on Tau fibrils may be assessed through multiple analytical approaches including changes in C-alpha root mean square deviation (RMSD) and calculation of interaction energy between MAP2-Tau or Tau-Tau layers. The C-alpha RMSD provides a measure of structural deviation from the starting configuration over the course of the simulation, with low RMSD values indicating stable maintenance of the initial binding pose. The interaction energy calculations provide quantitative assessment of the strength of intermolecular contacts between adjacent protein layers in the fibril complex.

The systematic computational approach enables assessment of binding potential and stability for multiple polypeptide variants or multiple disease-associated fibril conformations without requiring synthesis and experimental characterization of each variant. The microsecond scale simulations provide information about binding stability over timescales relevant to biological processes.

The computational workflow enables systematic comparison of binding properties across different disease-associated fibril conformations, informing selection of constructs with broad-spectrum or disease-specific activity profiles. The atomistic detail provided by molecular dynamics simulations enables identification of specific residue contacts and interaction networks that contribute to binding stability, guiding rational design of sequence modifications.

The workflow shown in FIG. 8B begins with the preparation of two test tubes containing different reaction mixtures. A first test tube contains tissue extracts combined with Tau protein and Thioflavin T (ThT). A second test tube contains tissue extracts combined with Tau protein, Thioflavin T, and MAP2. The parallel preparation of reaction mixtures with and without MAP2 enables direct comparison of Tau aggregation behavior in the presence and absence of the polypeptide capper, providing controlled assessment of inhibitory activity.

With continued reference to FIG. 8B, the contents of the test tubes are then transferred to set up reactions in a 96-well plate. The 96-well plate format provides a platform for conducting multiple parallel experiments, enabling simultaneous assessment of multiple biological replicates, technical replicates, or experimental conditions within a single assay run. The multi-well format enables high-throughput screening of polypeptide variants or concentration-response relationships while maintaining consistent assay conditions across all wells.

The 96-well plate shown in FIG. 8B is then placed into an RT-QuIC assay instrument. The RT-QuIC instrument performs Real-Time Quaking-Induced Conversion to amplify Tau proteins from proteopathic seeds present in processed disease brain biospecimens into long fibrils. The assay involves repeated cycles of shaking the reaction mixture followed by quiescent phases. In some cases, the assay may involve cycles of one minute of double orbital shaking at 400 rpm followed by nine minutes of resting phase. The cyclic shaking and resting protocol promotes fibril fragmentation during shaking phases, generating new fibril ends that serve as templates for monomer recruitment during quiescent phases, thereby accelerating the amplification process.

As further shown in FIG. 8B, following the RT-QuIC assay, the workflow branches into three parallel analysis pathways that provide complementary information about fibril formation and inhibitory activity. The three analysis pathways include fluorescence kinetics analysis, sedimentation assays, and electron microscopy visualization.

A first pathway shown in FIG. 8B involves fluorescence kinetics analysis, where the formation of fibrils is determined in real time by measuring Thioflavin T fluorescence over time. The fluorescence kinetics pathway is represented by a graph showing fluorescence intensity plotted against time with characteristic sigmoidal curves. ThT dye binds to beta-sheet rich fibrillar structures and exhibits enhanced fluorescence upon binding, providing a real-time readout of fibril formation during the assay. The sigmoidal kinetic profile reflects the nucleation-dependent polymerization mechanism of Tau fibril growth, with an initial lag phase corresponding to seed activation, followed by rapid growth during the exponential phase, and eventual plateau as monomer substrate becomes depleted.

A second pathway shown in FIG. 8B involves sedimentation assays, where the reaction products are subjected to ultracentrifugation to separate pellet and supernatant fractions. The sedimentation pathway includes analysis by gel electrophoresis to quantify the distribution of protein between soluble and insoluble fractions. The pellet fraction represents insoluble fibrillar aggregates that sediment during ultracentrifugation, while the supernatant fraction represents soluble monomeric protein that remains in solution. Densitometric analysis of gel bands enables quantitative determination of the percent distribution between pellet and supernatant fractions, allowing calculation of inhibition efficiency.

A third pathway shown in FIG. 8B involves electron microscopy visualization, which provides direct imaging of fibril formation or absence thereof in the reaction products. Electron microscopy enables assessment of fibril morphology independent of dye binding properties or sedimentation behavior, providing visual confirmation of whether fibrillar structures are present in samples. The morphological information obtained from electron microscopy images may reveal characteristics of fibril structure including length distribution, branching patterns, and association into higher-order assemblies.

The workflow enables assessment of the inhibitory effects of MAP2 proteins through qualitative and quantitative assays. Changes in fibril formation may be determined through comparison of reactions with and without MAP2 across all three analytical pathways. The correlation of results across fluorescence kinetics, sedimentation analysis, and electron microscopy imaging provides multiple independent lines of evidence for assessing polypeptide inhibitory activity against Tau aggregation from disease brain-derived seeds.

The comprehensive experimental workflow enables characterization of polypeptide inhibitory activity through multiple complementary methods. The fluorescence kinetics pathway provides real-time monitoring of fibril formation, the sedimentation pathway provides quantitative measurement of the physical state of Tau protein, and the electron microscopy pathway provides direct visualization of fibril morphology. The combination of three independent analytical approaches enables robust conclusions about inhibitory activity.

The 96-well plate format enables parallel assessment of multiple conditions within a single assay run, facilitating systematic comparison of inhibitory activity across polypeptide variants generated through mutagenesis or other sequence modification approaches.

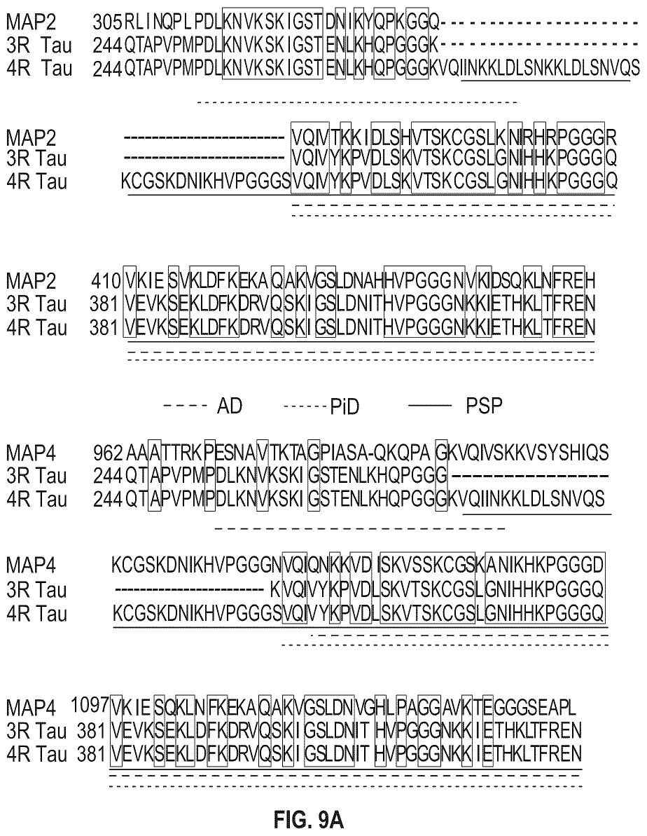

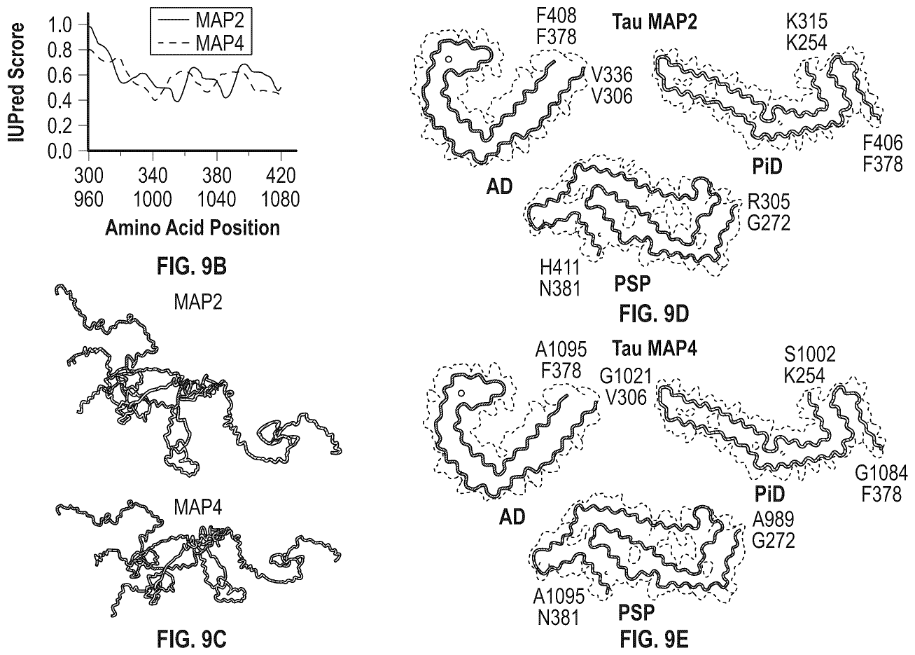

Referring to FIG. 9, a multi-panel diagram illustrates sequence analysis and structural modeling of microtubule-associated proteins MAP2 and MAP4 in relation to Tau protein. FIG. 9 presents five panels designated as panels a, b, c, d, and e, each providing different analytical perspectives on the sequence and structural characteristics of MAP proteins and their relationship to Tau protein.

Panel a of FIG. 9 shows a sequence alignment of repeat regions of 3R/4R Tau with MAP2 and MAP4. Shaded regions within the alignment indicate high conservation of amino acids between the MAP proteins and Tau variants. Lines positioned below the alignment indicate regions in Tau that form the amyloid core in different tauopathies, with dashed, dotted, and solid lines corresponding to AD, PiD, and PSP tauopathies respectively. The sequence alignment demonstrates that MAP2 and MAP4 share substantial sequence similarity with the Tau regions that form the structured core of pathological filaments, providing the molecular basis for homology-based engagement with fibril surfaces.

With continued reference to FIG. 9, panel b presents a graph showing sequence-based intrinsic disorder analysis of MAP proteins. The graph plots IUPred Score on the vertical axis ranging from 0.0 to 1.0, with amino acid position displayed on the horizontal axis. Curves for both MAP2 and MAP4 are displayed, with specific residue positions labeled including F405, F373, V338, V306, K315, K254, F406, F378, R305, G272, H411, and N381. The IUPred scores for both MAP2 and MAP4 remain elevated across the analyzed regions, indicating that the repeat regions of MAP proteins exhibit intrinsically disordered character in the absence of a binding partner.

The intrinsically disordered nature of MAP proteins provides advantages in various embodiments for engaging with Tau fibril surfaces. Intrinsically disordered proteins lack stable three-dimensional structure in isolation but may adopt defined conformations upon binding to structured partners. The disordered character of MAP2 and MAP4 repeat regions enables conformational flexibility that allows the proteins to adapt to different disease-associated Tau folds. The ability to undergo disorder-to-order transitions upon fibril engagement enables MAP proteins to adopt conformations complementary to the specific structural topology of each disease-associated fibril type.

As further shown in FIG. 9, panel c displays ab-initio modeling of MAP proteins, depicting the disordered structural nature of MAP2 as ribbon representations. The ab-initio modeling results confirm the intrinsically disordered character predicted by the IUPred analysis, showing that MAP2 does not adopt a stable folded structure in the absence of a template. The disordered conformations depicted in panel c represent the conformational ensemble that MAP proteins may sample in solution prior to engagement with Tau fibril surfaces.

Panels d and e of FIG. 9 illustrate homology-based modeling of MAP2 and MAP4 respectively into different types of disease-associated Tau folds. In panels d and e, colored chains representing the modeled MAP proteins are superimposed over Tau fibrils shown as gray surfaces from AD, PiD, and PSP. The structural representations include labeled residue positions such as A1065, G1021, V306, S1002, K254, G1084, F378, A080, G272, A1065, and N381. The three-dimensional superposition demonstrates that both MAP2 and MAP4 may acquire different types of disease-specific Tau folds through conformational adaptation enabled by the sequence similarity revealed in panel a.

The polypeptide may be a truncated version of MAP2, MAP4, or Tau protein that encompasses only the core region responsible for fibril interaction. The truncated constructs may use specific regions corresponding to the repeat domains that contribute to the high sequence similarity with 3R or 4R Tau. For MAP2, the truncated construct may encompass residues R305-E410 of UniProt P11137-2, or equivalent regions in other MAP2 isoforms. For MAP4, the truncated construct may encompass residues A962-L1097 of UniProt P27816-1, or equivalent regions in other MAP4 isoforms. The truncated versions retain the structured core that engages with disease-associated fibril ends while excluding regions that do not contribute to fibril interaction.

The combination of sequence analysis and structural modeling shown in FIG. 9 informs understanding and optimization of polypeptide capping activity. The sequence alignment identifies conserved residue positions that may contribute to fibril engagement, the disorder analysis confirms conformational flexibility, and the homology-based modeling demonstrates that MAP proteins may adopt disease-specific folds upon engagement with fibril surfaces.

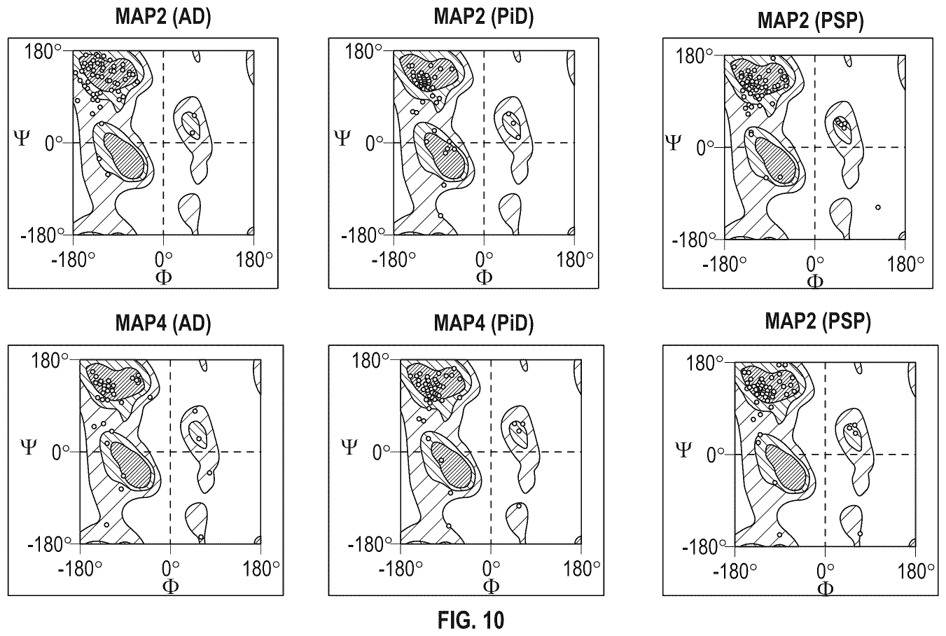

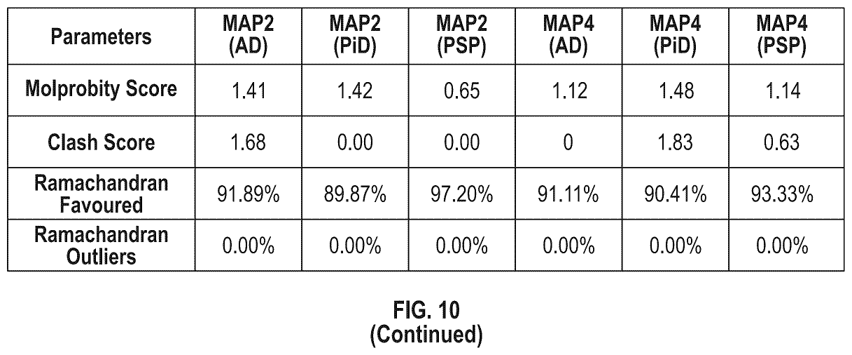

Referring to FIG. 10, Ramachandran plots and structural analysis parameters for homology modeling of MAP2 and MAP4 proteins into different disease-associated Tau folds are presented. FIG. 10 displays six Ramachandran plots arranged in two rows, with the top row displaying plots for MAP2 modeled into AD, PiD, and PSP folds from left to right, and the bottom row displaying plots for MAP4 modeled into the same three disease folds. Each plot shows the distribution of phi and psi dihedral angles for the modeled protein structures, with data points clustered in favored regions indicated by shaded areas.

The Ramachandran plots shown in FIG. 10 provide assessment of backbone geometry for the modeled MAP2 and MAP4 structures in each disease-associated fold. The phi and psi dihedral angles define the backbone conformation of each amino acid residue in the protein chain, and the distribution of these angles across the Ramachandran plot indicates whether the modeled structures adopt geometrically favorable conformations. Data points clustered within the shaded favored regions indicate residues with backbone geometries consistent with well-folded protein structures, while data points outside the favored regions may indicate strained or unfavorable backbone conformations.

With continued reference to FIG. 10, a table positioned below the Ramachandran plots presents structural quality parameters for each of the six modeled structures. The table includes MolProbity Score values ranging from 0.65 to 1.48 across the six modeled structures. The MolProbity score represents a combined protein quality score that reflects the crystallographic resolution at which such quality would be expected, with lower scores indicating higher structural quality. MolProbity scores below 2.0 are considered favorable for structures with acceptable backbone geometry and minimal steric clashes.

The table in FIG. 10 also presents Clash Score values ranging from 0 to 1.83 for the six modeled structures. The clash score represents van der Waals sphere overlap of greater than 0.4 Angstroms between non-bonded atoms in the structure. Clash scores below 5 are considered favorable for structures with minimal steric clashes between atoms. The low clash scores observed across all six modeled MAP2 and MAP4 structures indicate that the modeled conformations do not exhibit unfavorable atomic overlaps that would indicate structural strain or modeling artifacts.

As further shown in FIG. 10, the table presents Ramachandran Favoured percentages ranging from 89.87% to 97.20% for the six modeled structures. The Ramachandran favored percentage indicates the proportion of residues with backbone dihedral angles falling within the most favorable regions of the Ramachandran plot. Higher percentages indicate that a greater proportion of residues adopt geometrically favorable backbone conformations. The Ramachandran favored percentages above 89% observed across all six modeled structures indicate that the modeled MAP2 and MAP4 conformations exhibit backbone geometries consistent with well-folded protein structures.

The table in FIG. 10 also presents Ramachandran Outliers percentages of 0.00% for all six modeled structures. Ramachandran outliers represent residues with backbone dihedral angles falling in disallowed regions of the Ramachandran plot, indicating geometrically unfavorable or strained backbone conformations. The absence of Ramachandran outliers across all six modeled structures indicates that none of the residues in the modeled MAP2 and MAP4 conformations adopt disallowed backbone geometries.

The polypeptide may be validated using Ramachandran analysis and MolProbity scoring to assess structural quality of modeled conformations. The structural quality validation approach enables assessment of whether modeled polypeptide conformations exhibit geometrically favorable backbone geometries and minimal steric clashes prior to molecular dynamics simulation or experimental validation. The MolProbity integration within SwissModel workspace or similar homology modeling platforms provides automated assessment of structural quality parameters during the modeling process.

The structural quality validation approach enables assessment of whether modeled polypeptide conformations exhibit geometrically favorable backbone geometries and minimal steric clashes prior to molecular dynamics simulation or experimental validation.

The structural quality parameters shown in FIG. 10 indicate that the modeled MAP2 and MAP4 structures exhibit correct fold architectures when modeled into AD, PiD, and PSP disease-associated Tau folds. The favorable MolProbity scores, low clash scores, high Ramachandran favored percentages, and absence of Ramachandran outliers indicate that both MAP2 and MAP4 may favorably adopt various disease-associated Tau structural folds without introducing geometric strain or steric clashes.

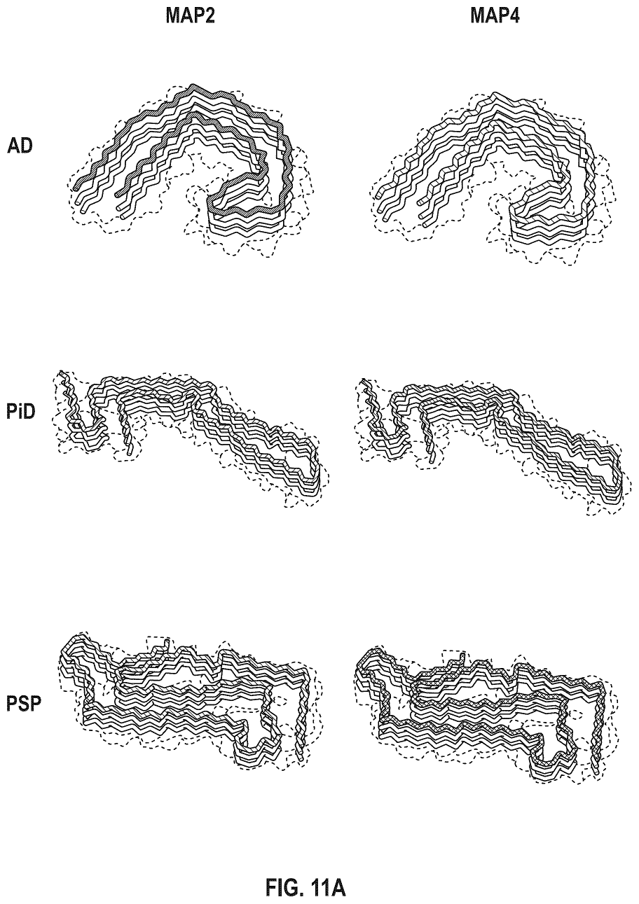

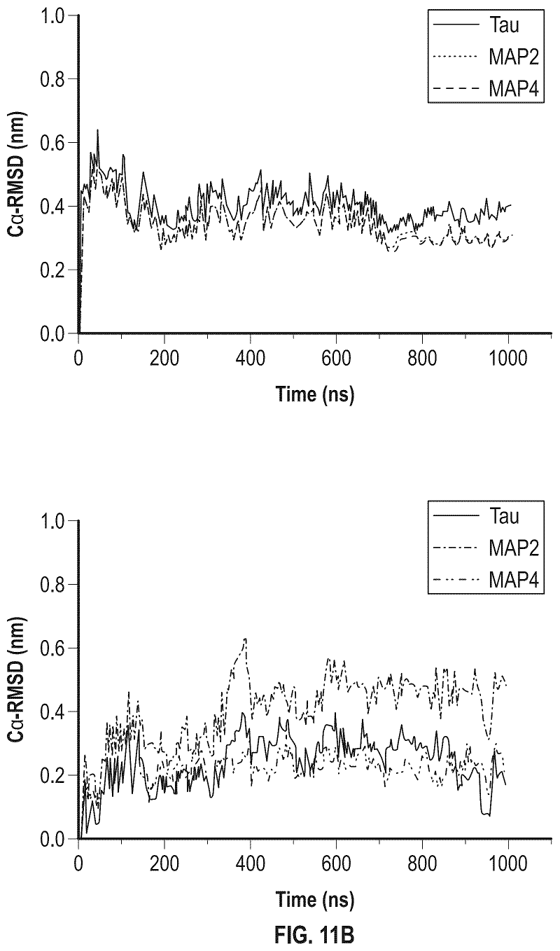

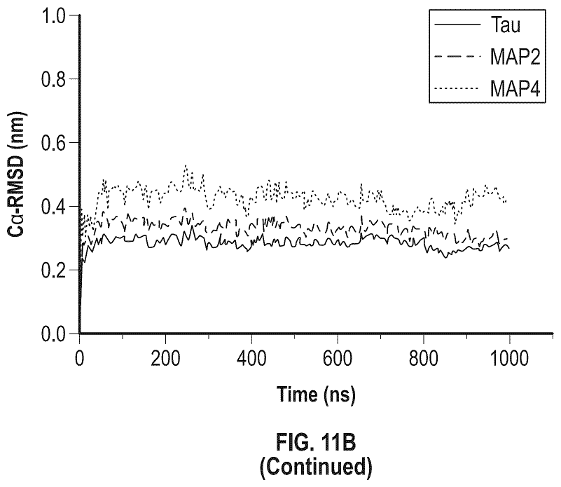

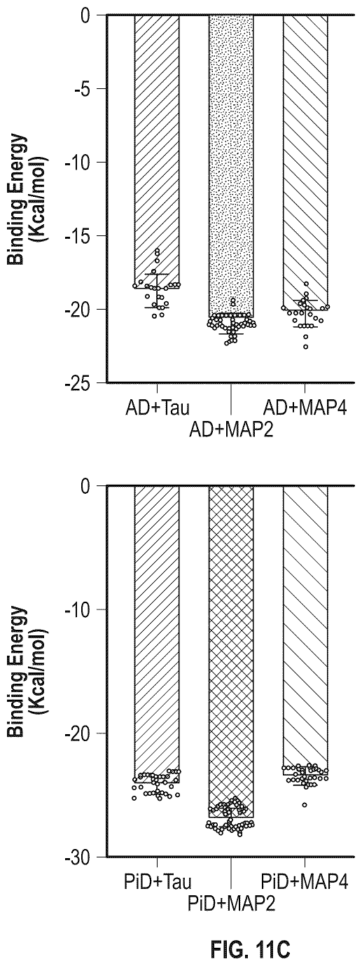

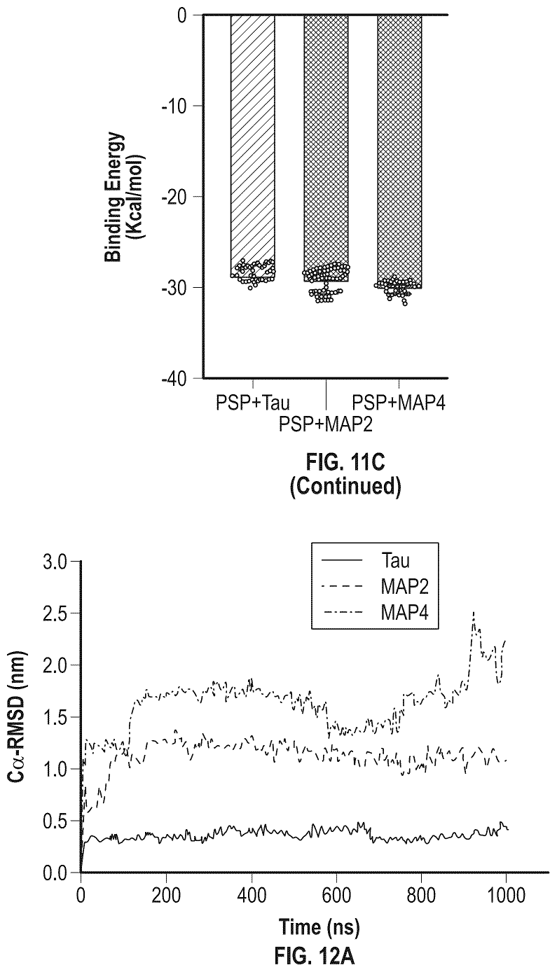

Referring to FIG. 11, molecular dynamics simulations and binding energy analyses of MAP2 and MAP4 docked with Tau fibrils from three different tauopathies are presented. FIG. 11 is organized into three columns labeled a, b, and c, with three rows corresponding to Alzheimer's disease (AD), Pick's disease (PiD), and progressive supranuclear palsy (PSP) respectively. The three-column organization enables systematic comparison of structural representations, temporal stability measurements, and binding energy quantification across the three disease-associated fibril types.

Column a of FIG. 11 shows structural representations of MAP2 and MAP4 chains docked onto disease-associated Tau fibril folds. In each structural representation, the MAP2 chains are shown in darker coloring and MAP4 chains in lighter coloring, with the underlying Tau fibril structures depicted in gray. The AD row displays orange-red colored MAP chains positioned along the characteristic curved conformation of the Alzheimer's disease-associated Tau fibril. The PiD row displays blue-purple colored MAP chains following the meandering path of the Pick's disease-associated 3R Tau fibril structure. The PSP row displays green colored MAP chains conforming to the more elongated architecture associated with progressive supranuclear palsy Tau fibrils. The structural representations demonstrate that both MAP2 and MAP4 may adopt conformations complementary to the fibril surface across all three disease-associated fold types.

With continued reference to FIG. 11, column b presents graphs showing temporal changes in C-alpha root mean square deviation (Ca-RMSD) measured in nanometers over a simulation time of 1000 nanoseconds for each disease type. Each graph contains three traces representing Tau shown in black or gray, MAP2 shown in the darker disease-specific color, and MAP4 shown in the lighter disease-specific color. The Ca-RMSD values provide a measure of structural deviation from the starting configuration over the course of the simulation, with low RMSD values indicating stable maintenance of the initial binding pose and high RMSD values indicating structural drift or dissociation from the fibril surface.

The Ca-RMSD traces in column b of FIG. 11 demonstrate that MAP2 and MAP4 maintain stable association with Tau fibril surfaces throughout the 1000 nanosecond simulation period across all three tauopathies. The RMSD values for MAP2-Tau and MAP4-Tau complexes generally stabilize between 0.2 and 0.6 nanometers, comparable to the RMSD values observed for native Tau-Tau interactions within the fibril structures. The stable RMSD profiles indicate that MAP2 and MAP4 remain engaged with the fibril surface without dissociating or undergoing large conformational changes that would disrupt the capping interaction. The comparable RMSD values between MAP-Tau and Tau-Tau interactions suggest that MAP proteins integrate into the fibril structure in a manner similar to native Tau monomers.

As further shown in FIG. 11, column c displays bar graphs showing binding energy analyses in kilocalories per mole for the interactions between Tau-Tau, MAP2-Tau, and MAP4-Tau in each disease-associated fibril complex. The binding energies were calculated using protein-protein contacts-based approaches, with values averaged over portions of the simulation trajectory to obtain representative measurements that account for conformational fluctuations. Error bars on each measurement indicate standard deviation, demonstrating variability in the calculated binding energies across the analyzed trajectory frames.

The binding energy values shown in column c of FIG. 11 range from approximately −5 to −40 kilocalories per mole across the different chain pair interactions. The binding energy values demonstrate that MAP2 and MAP4 interact with Tau fibrils with comparable or higher affinity relative to Tau-Tau interactions across all three tauopathy-associated fibril conformations. In the AD fibril complex, the MAP2-Tau and MAP4-Tau binding energies are comparable to or exceed the Tau-Tau layer binding energies, indicating that MAP proteins engage with the AD fibril surface with affinity sufficient to compete with native Tau monomer recruitment. Similar patterns are observed in the PiD and PSP fibril complexes, where MAP2 and MAP4 exhibit binding energies comparable to the native Tau-Tau interactions within each respective fibril structure.

The polypeptide forms a terminal layer cap on the fibril but may not stack to form multiple layers, preventing elongation. The molecular dynamics simulations and binding energy analyses support a capping mechanism whereby MAP2 and MAP4 engage with the fibril end through stable, high-affinity interactions that occupy the terminal binding site without enabling further monomer recruitment. The comparable binding energies between MAP-Tau and Tau-Tau interactions indicate that MAP proteins may effectively compete with Tau monomers for binding sites at fibril ends. However, the inability of MAP proteins to support stacking of additional layers prevents the elongation process that would otherwise propagate fibril growth.

The stable binding demonstrated in FIG. 11 indicates that polypeptide constructs may remain engaged with fibril ends for durations sufficient to interfere with Tau monomer recruitment in biological contexts. The comparable binding affinities across AD, PiD, and PSP fibril complexes indicate that a single polypeptide construct may exhibit broad-spectrum capping activity against multiple tauopathies characterized by different fibril conformations.

The quantitative binding energy values enable comparison of interaction strengths across different polypeptide variants or different disease-associated fibril conformations, informing selection of constructs with favorable binding properties. The combination of stable RMSD profiles and favorable binding energies across all three tauopathies supports characterization of MAP2 and MAP4 as broad-spectrum fibril cappers.

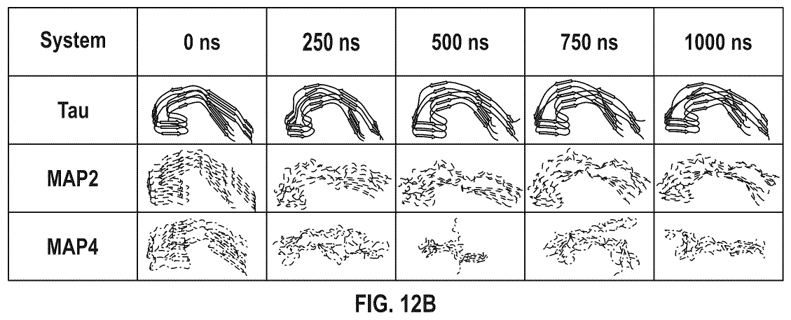

Referring to FIG. 12, molecular dynamics simulations of MAP2 and MAP4 in an Alzheimer's disease fibril fold are presented. FIG. 12 is organized into two panels designated as panel a and panel b, providing complementary analytical perspectives on the stability behavior of MAP proteins when modeled into the AD-associated Tau fibril conformation as multi-layered assemblies rather than as single-layer caps on existing Tau fibrils.

Panel a of FIG. 12 shows a graph plotting Ca-RMSD in nanometers on the vertical axis against time in nanoseconds on the horizontal axis, with the simulation extending from 0 to 1000 nanoseconds. Three traces are displayed in the graph, representing Tau shown in grey, MAP2 shown in red, and MAP4 shown in orange. The Ca-RMSD values provide a measure of structural deviation from the starting configuration over the course of the simulation, enabling assessment of whether the modeled protein assemblies maintain their initial fold architecture or undergo structural destabilization during the simulation period.

With continued reference to panel a of FIG. 12, the Tau trace remains relatively stable at approximately 0.3 to 0.5 nanometers throughout the 1000 nanosecond simulation period. The stable RMSD profile for Tau indicates that the native Tau fibril assembly maintains the AD-associated fold architecture without undergoing substantial structural deviation from the starting configuration. The low RMSD values observed for Tau throughout the simulation demonstrate that the AD fibril fold represents a stable structural state for Tau protein assemblies under the simulation conditions employed.

The MAP2 trace shown in panel a of FIG. 12 exhibits an increase in Ca-RMSD values compared to the starting structure, rising to approximately 1.0 to 1.5 nanometers with fluctuations over the simulation period. The elevated RMSD values for MAP2 indicate that the MAP2 assembly modeled into the AD fibril fold undergoes structural deviation from the initial configuration during the simulation. The increase in RMSD for MAP2 compared to the stable Tau trace demonstrates that MAP2 does not maintain the AD-associated fold architecture as stably as native Tau protein.

As further shown in panel a of FIG. 12, the MAP4 trace exhibits a more dramatic increase in Ca-RMSD values compared to both Tau and MAP2. The MAP4 RMSD values rise to approximately 2.0 to 2.5 nanometers during the simulation period, indicating substantial structural deviation from the starting AD fibril fold configuration. The elevated RMSD values for MAP4 demonstrate that MAP4 assemblies modeled into the AD fibril fold undergo more pronounced structural destabilization compared to MAP2 assemblies under the same simulation conditions.

Panel b of FIG. 12 presents a table showing structural snapshots extracted from simulation trajectories at time points of 0, 250, 500, 750, and 1000 nanoseconds for three systems: Tau, MAP2, and MAP4. The structural snapshots provide visual representation of the conformational changes that occur during the simulation period, enabling assessment of how the three-dimensional architecture of each protein assembly evolves over time.

With continued reference to panel b of FIG. 12, the Tau row displays grey ribbon structures that maintain a consistent curved fibril fold architecture across all five time points from 0 to 1000 nanoseconds. The structural snapshots for Tau demonstrate that the native Tau fibril assembly retains the characteristic AD-associated fold topology throughout the simulation period, with the curved conformation and inter-layer packing interactions preserved at each extracted time point. The maintenance of fibril architecture in the Tau snapshots correlates with the stable RMSD profile observed in panel a.

The MAP2 row in panel b of FIG. 12 displays red ribbon structures that show progressive structural changes and deviation from the initial fold over time. At the 0 nanosecond time point, the MAP2 structure exhibits the AD-associated fold architecture similar to the Tau template used for homology modeling. At subsequent time points of 250, 500, 750, and 1000 nanoseconds, the MAP2 structures display increasing deviation from the initial fold topology, with visible alterations in the backbone trajectory and inter-layer organization. The progressive structural changes observed in the MAP2 snapshots correlate with the elevated RMSD values shown in panel a.

As further shown in panel b of FIG. 12, the MAP4 row displays orange ribbon structures that demonstrate substantial structural destabilization and loss of the original fibril fold architecture as the simulation progresses. At the 0 nanosecond time point, the MAP4 structure exhibits the AD-associated fold architecture derived from the homology modeling process. At subsequent time points, the MAP4 structures display marked destabilization of the tertiary architecture, with the characteristic curved conformation of the AD fold becoming disrupted and the inter-layer organization becoming disordered. The pronounced structural destabilization observed in the MAP4 snapshots correlates with the elevated RMSD values exceeding 2.0 nanometers shown in panel a.

The Ca-RMSD and structural analysis presented in FIG. 12 indicate that Tau may favorably remain in the Alzheimer's disease fold while MAP proteins modeled into the same fold show altered structural topology compared to the starting model or Tau fibril. The inability of MAP2 and MAP4 to sustain the AD-associated fold architecture when assembled as multi-layered structures indicates that MAP proteins may not template further growth of MAP or Tau monomers once engaged with a fibril end. The structural destabilization observed for MAP assemblies contrasts with the stable capping behavior demonstrated when MAP proteins are docked as single layers onto existing Tau fibril surfaces.

The polypeptide may cap fibril ends but may not support elongation through stacking of additional layers. The molecular dynamics simulations of MAP assemblies in the AD fold demonstrate that while MAP proteins may adopt the disease-associated conformation when engaged with a Tau fibril surface as a terminal layer, MAP proteins may not maintain the fold architecture when assembled as multi-layered structures that would be required for fibril elongation. The inability to sustain the disease-associated fold in multi-layered assemblies provides a mechanistic basis for the capping behavior observed in experimental assays, where MAP proteins inhibit Tau aggregation without themselves forming fibrillar deposits.

The structural destabilization observed in FIG. 12 indicates that MAP-based polypeptide constructs may function as terminal caps that block fibril elongation without propagating fibril growth. The capping mechanism may prevent recruitment of additional Tau monomers onto fibril ends while avoiding the formation of heteromolecular MAP-Tau fibril assemblies that could potentially contribute to pathology.

The differential stability behavior between single-layer capping and multi-layer assembly indicates that MAP proteins may engage with fibril ends through favorable interactions while lacking the inter-layer contacts required for propagating fibril growth. The sequence differences between MAP proteins and Tau at positions contributing to inter-layer contacts may account for this differential stability behavior.

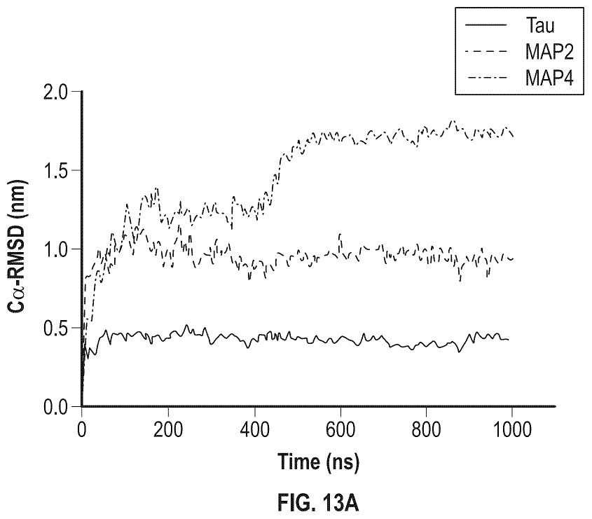

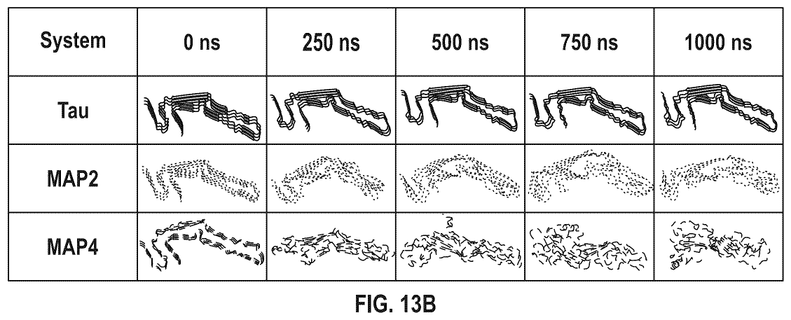

Referring to FIG. 13, molecular dynamics simulations of MAP2 and MAP4 in a Pick's disease fibril fold are presented. FIG. 13 is organized into two panels designated as panel a and panel b, providing complementary analytical perspectives on the stability behavior of MAP proteins when modeled into the PiD-associated Tau fibril conformation as multi-layered assemblies rather than as single-layer caps on existing Tau fibrils.

Panel a of FIG. 13 shows a graph illustrating temporal changes in Ca-RMSD over time from 0 to 1000 nanoseconds for three systems: Tau shown in black, MAP2 shown in dark blue, and MAP4 shown in light blue. The vertical axis displays Ca-RMSD values in nanometers, enabling assessment of structural deviation from the starting configuration over the course of the simulation. The Ca-RMSD measurements provide quantitative assessment of whether the modeled protein assemblies maintain their initial PiD-associated fold architecture or undergo structural destabilization during the microsecond-scale simulation period.

With continued reference to panel a of FIG. 13, the Tau trace remains relatively stable at approximately 0.5 nm throughout the 1000 nanosecond simulation period. The stable RMSD profile for Tau indicates that the native Tau fibril assembly maintains the PiD-associated fold architecture without undergoing substantial structural deviation from the starting configuration. The low RMSD values observed for Tau throughout the simulation demonstrate that the PiD fibril fold represents a stable structural state for 3R Tau protein assemblies under the simulation conditions employed.

The MAP2 trace shown in panel a of FIG. 13 exhibits an initial increase in Ca-RMSD values and stabilizes around 1.0 nm over the simulation period. The elevated RMSD values for MAP2 compared to the Tau trace indicate that the MAP2 assembly modeled into the PiD fibril fold undergoes structural deviation from the initial configuration during the simulation. The increase in RMSD for MAP2 compared to the stable Tau trace demonstrates that MAP2 does not maintain the PiD-associated fold architecture as stably as native 3R Tau protein when assembled as multi-layered structures.

As further shown in panel a of FIG. 13, the MAP4 trace demonstrates the highest RMSD values among the three systems examined. The MAP4 RMSD values rise sharply in the first 200 nanoseconds and fluctuate around 1.5 to 2.0 nm for the remainder of the simulation period. The elevated RMSD values for MAP4 indicate substantial structural deviation from the starting PiD fibril fold configuration, demonstrating that MAP4 assemblies modeled into the PiD fibril fold undergo more pronounced structural destabilization compared to MAP2 assemblies under the same simulation conditions.

Panel b of FIG. 13 presents a table showing structural snapshots extracted from respective simulation trajectories at five time points: 0 ns, 250 ns, 500 ns, 750 ns, and 1000 ns. The structural snapshots provide visual representation of the conformational changes that occur during the simulation period for each of the three protein systems, enabling assessment of how the three-dimensional architecture of each protein assembly evolves over time in the context of the PiD-associated fold.

With continued reference to panel b of FIG. 13, the Tau row displays gray-colored structures that maintain their fibril fold architecture throughout the simulation period. At each of the five extracted time points from 0 to 1000 nanoseconds, the Tau structures retain the characteristic meandering conformation of the Pick's disease-associated 3R Tau fibril fold. The maintenance of fibril architecture in the Tau snapshots correlates with the stable RMSD profile observed in panel a, demonstrating that native Tau assemblies sustain the PiD fold topology under the simulation conditions.

The MAP2 row in panel b of FIG. 13 shows purple-colored structures that exhibit progressive structural changes and altered topology compared to the starting model. At the 0 nanosecond time point, the MAP2 structure exhibits the PiD-associated fold architecture derived from the homology modeling process. At subsequent time points of 250, 500, 750, and 1000 nanoseconds, the MAP2 structures display increasing deviation from the initial fold topology, with visible alterations in the backbone trajectory and inter-layer organization. The progressive structural changes observed in the MAP2 snapshots correlate with the elevated RMSD values shown in panel a.

As further shown in panel b of FIG. 13, the MAP4 row displays blue-colored structures that demonstrate marked destabilization of tertiary architecture over the simulation time course. At the 0 nanosecond time point, the MAP4 structure exhibits the PiD-associated fold architecture similar to the Tau template used for homology modeling. At subsequent time points, the MAP4 structures display pronounced disruption of the characteristic PiD fold conformation, with the inter-layer organization becoming increasingly disordered. The structural destabilization observed in the MAP4 snapshots correlates with the elevated RMSD values exceeding 1.5 nm shown in panel a.

The Ca-RMSD and structural analysis presented in FIG. 13 indicate that Tau may favorably remain in the PiD fold while MAP proteins modeled into PiD folds show altered structural topology compared to the starting model or Tau PiD fibril. The inability of MAP2 and MAP4 to sustain the PiD-associated fold architecture when assembled as multi-layered structures indicates that MAP proteins may not template further growth of MAP or Tau monomers once engaged with a fibril end in the context of Pick's disease-associated fibrils.

The polypeptide may cap PiD fibril ends but may not support elongation through stacking of additional layers in the PiD fold context. The molecular dynamics simulations of MAP assemblies in the PiD fold demonstrate that while MAP proteins may adopt the disease-associated conformation when engaged with a Tau fibril surface as a terminal layer, MAP proteins may not maintain the fold architecture when assembled as multi-layered structures that would be required for fibril elongation. The inability to sustain the disease-associated fold in multi-layered assemblies provides a mechanistic basis for the capping behavior observed in experimental assays with PiD brain-derived seeds.

The structural destabilization observed in FIG. 13 indicates that MAP-based polypeptide constructs may function as terminal caps that block fibril elongation in 3R tauopathies without propagating fibril growth.

The stable binding of MAP proteins to PiD Tau fibril surfaces as terminal layers, combined with the inability to sustain multi-layered assemblies, indicates that MAP proteins may engage with 3R fibril ends through favorable interactions while lacking the inter-layer contacts required for propagating fibril growth in the PiD fold architecture.

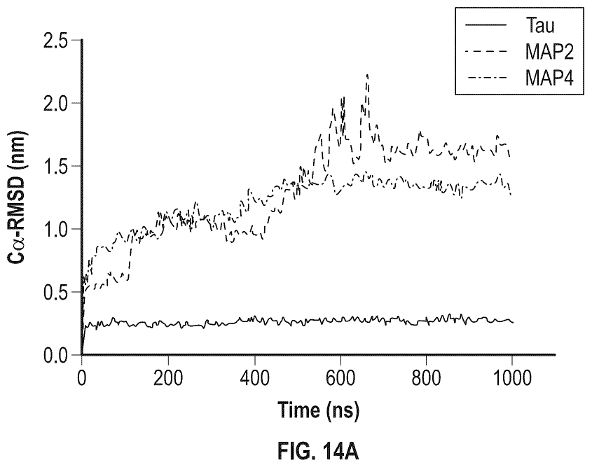

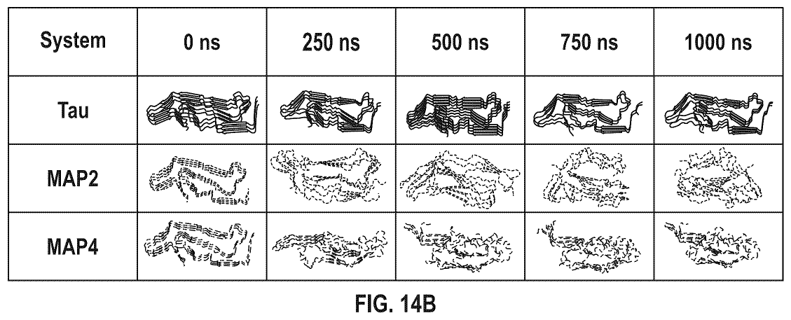

Referring to FIG. 14, molecular dynamics simulations of MAP2 and MAP4 in a progressive supranuclear palsy fibril fold are presented. FIG. 14 is organized into two panels designated as panel a and panel b, providing complementary analytical perspectives on the stability behavior of MAP proteins when modeled into the PSP-associated Tau fibril conformation as multi-layered assemblies rather than as single-layer caps on existing Tau fibrils.

Panel a of FIG. 14 shows a graph of temporal changes in Ca-RMSD measured in nanometers over time measured in nanoseconds from 0 to 1000 ns. Three traces are displayed in the graph, representing Tau in grey, MAP2 in dark green, and MAP4 in light green. The vertical axis displays Ca-RMSD values in nanometers, enabling assessment of structural deviation from the starting configuration over the course of the simulation. The Ca-RMSD measurements provide quantitative assessment of whether the modeled protein assemblies maintain their initial PSP-associated fold architecture or undergo structural destabilization during the microsecond-scale simulation period.

With continued reference to panel a of FIG. 14, the Tau trace remains relatively stable at approximately 0.3 nm throughout the 1000 nanosecond simulation period. The stable RMSD profile for Tau indicates that the native Tau fibril assembly maintains the PSP-associated fold architecture without undergoing substantial structural deviation from the starting configuration. The low RMSD values observed for Tau throughout the simulation demonstrate that the PSP fibril fold represents a stable structural state for 4R Tau protein assemblies under the simulation conditions employed.

The MAP2 and MAP4 traces shown in panel a of FIG. 14 exhibit increased RMSD values compared to the Tau trace, rising to approximately 1.5 to 2.0 nanometers during the simulation period. The elevated RMSD values for both MAP2 and MAP4 indicate structural deviation from the starting PSP fibril fold configuration, demonstrating that MAP protein assemblies modeled into the PSP fibril fold undergo structural destabilization compared to native Tau assemblies under the same simulation conditions. The increase in RMSD for both MAP proteins compared to the stable Tau trace demonstrates that MAP2 and MAP4 do not maintain the PSP-associated fold architecture as stably as native 4R Tau protein when assembled as multi-layered structures.

Panel b of FIG. 14 presents a table showing structures of Tau, MAP2, and MAP4 in PSP folds extracted from respective simulation trajectories at time points of 0 ns, 250 ns, 500 ns, 750 ns, and 1000 ns. The structural snapshots provide visual representation of the conformational changes that occur during the simulation period for each of the three protein systems, enabling assessment of how the three-dimensional architecture of each protein assembly evolves over time in the context of the PSP-associated fold.

With continued reference to panel b of FIG. 14, the Tau structures shown in grey maintain their fibril architecture throughout the simulation period. At each of the five extracted time points from 0 to 1000 nanoseconds, the Tau structures retain the characteristic elongated conformation of the progressive supranuclear palsy-associated 4R Tau fibril fold. The maintenance of fibril architecture in the Tau snapshots correlates with the stable RMSD profile observed in panel a, demonstrating that native Tau assemblies sustain the PSP fold topology under the simulation conditions.

The MAP2 structures shown in dark green in panel b of FIG. 14 display progressively altered structural topology compared to the starting model over the course of the simulation. At the 0 nanosecond time point, the MAP2 structure exhibits the PSP-associated fold architecture derived from the homology modeling process. At subsequent time points of 250, 500, 750, and 1000 nanoseconds, the MAP2 structures display increasing deviation from the initial fold topology, with visible destabilization of the tertiary architecture. The progressive structural changes observed in the MAP2 snapshots correlate with the elevated RMSD values shown in panel a.

As further shown in panel b of FIG. 14, the MAP4 structures shown in light green demonstrate substantial structural destabilization and loss of the original fibril fold architecture as the simulation progresses. At the 0 nanosecond time point, the MAP4 structure exhibits the PSP-associated fold architecture similar to the Tau template used for homology modeling. At subsequent time points, the MAP4 structures display pronounced disruption of the characteristic PSP fold conformation, with the inter-layer organization becoming increasingly disordered. The structural destabilization observed in the MAP4 snapshots correlates with the elevated RMSD values shown in panel a.

The Ca-RMSD and structural analysis presented in FIG. 14 indicate that Tau may favorably remain in the PSP fold while MAP proteins modeled into PSP folds show altered structural topology compared to the starting model or Tau PSP fibril. The inability of MAP2 and MAP4 to sustain the PSP-associated fold architecture when assembled as multi-layered structures indicates that MAP proteins may not template further growth of MAP or Tau monomers once engaged with a fibril end in the context of progressive supranuclear palsy-associated fibrils.

The polypeptide may cap PSP fibril ends but may not support elongation through stacking of additional layers in the PSP fold context. The molecular dynamics simulations of MAP assemblies in the PSP fold demonstrate that while MAP proteins may adopt the disease-associated conformation when engaged with a Tau fibril surface as a terminal layer, MAP proteins may not maintain the fold architecture when assembled as multi-layered structures that would be required for fibril elongation. The inability to sustain the disease-associated fold in multi-layered assemblies provides a mechanistic basis for the capping behavior observed in experimental assays with PSP brain-derived seeds.

The structural destabilization observed in FIG. 14 indicates that MAP-based polypeptide constructs may function as terminal caps that block fibril elongation in 4R tauopathies without propagating fibril growth.

The stable binding of MAP proteins to PSP Tau fibril surfaces as terminal layers, combined with the inability to sustain multi-layered assemblies, indicates that MAP proteins may engage with 4R fibril ends through favorable interactions while lacking the inter-layer contacts required for propagating fibril growth in the PSP fold architecture.

The consistent pattern of structural destabilization observed across AD, PiD, and PSP fold contexts in FIGS. 12, 13, and 14 indicates that the capping mechanism may operate through a common structural basis regardless of the specific disease-associated fold conformation, supporting development of polypeptide constructs with activity against multiple tauopathies.

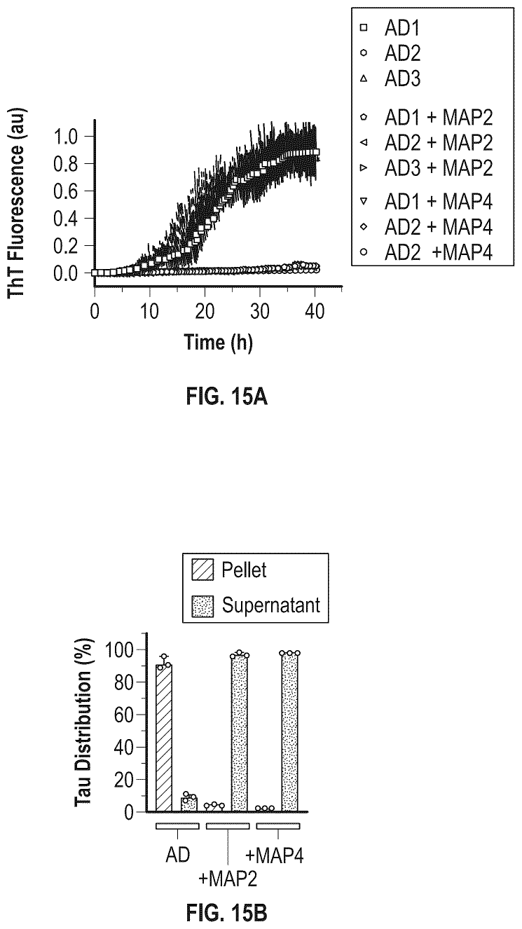

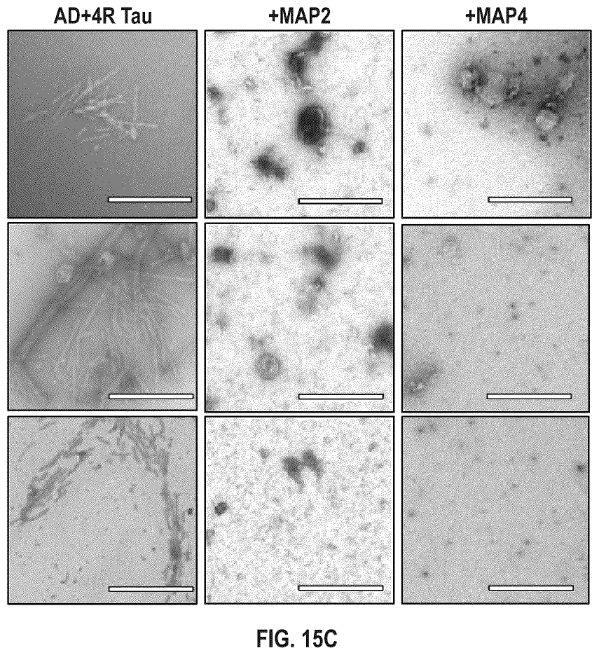

Referring to FIG. 15, experimental data demonstrating that MAP2 and MAP4 inhibit 4R Tau aggregation from Alzheimer's disease brain-derived samples is presented. FIG. 15 is organized into three panels designated as panel a, panel b, and panel c, providing complementary analytical perspectives on the inhibitory activity of MAP2 and MAP4 against Tau aggregation from AD brain extracts using ThT fluorescence kinetics, sedimentation analysis, and electron microscopy imaging.

Panel a of FIG. 15 shows primary Thioflavin T fluorescence data from RT-QuIC reactions of tissue extracts from three AD brains designated AD1, AD2, and AD3 with 4R Tau protein. Gray traces represent reactions with AD brain extracts and 4R Tau protein alone, while red traces represent reactions in the presence of MAP2 and orange traces represent reactions in the presence of MAP4. The reactions were carried out in triplicates from three different biological AD subjects, with shaded regions indicating standard error of mean. Across all three AD samples, the gray traces exhibit sigmoidal growth curves, while the red and orange traces remain at or near baseline fluorescence levels, indicating that both MAP2 and MAP4 inhibit 4R Tau aggregation from AD brain-derived seeds.

As further shown in FIG. 15, panel b presents bar graphs of quantitative analysis depicting Tau distribution as percentage in pellet and supernatant fractions for AD alone, AD with MAP2, and AD with MAP4 conditions, using the sedimentation assay methodology described above for FIG. 3B. Dark bars represent pellet fractions containing insoluble fibrillar aggregates, while light bars represent supernatant fractions containing soluble monomeric protein.

In the AD alone condition shown in panel b of FIG. 15, the pellet fraction approaches approximately 90 percent or greater while the supernatant fraction remains minimal at approximately 0 to 10 percent, indicating that 4R Tau monomers have been recruited onto the AD brain-derived seeds and converted into insoluble fibrillar species during the RT-QuIC reaction.

With continued reference to panel b of FIG. 15, in the AD with MAP2 and AD with MAP4 conditions, the distribution reverses such that the supernatant fraction approaches approximately 90 to 100 percent while the pellet fraction decreases to approximately 0 to 10 percent. The polypeptide inhibits aggregation of both 3R and 4R Tau isoforms from mixed tauopathies like Alzheimer's disease, as demonstrated by the inhibition of 4R Tau aggregation shown in FIG. 15 combined with the inhibition of 3R Tau aggregation from AD seeds demonstrated in previous figures.

Panel c of FIG. 15 shows electron microscopy images of fibril formation arranged in a three-by-three grid. The three columns represent AD plus 4R Tau, plus MAP2, and plus MAP4 conditions from left to right. The three rows represent AD1, AD2, and AD3 samples from top to bottom. The grid organization enables systematic comparison of fibril morphology across the three biological samples and three treatment conditions.

The left column of the grid shown in panel c of FIG. 15 displays images for AD samples with 4R Tau alone. The images in the left column show elongated fibrillar structures of varying lengths characteristic of Tau fibrils, with extended linear profiles visible against the background of the carbon-coated copper grid support. The presence of fibrillar structures in the left column correlates with the sigmoidal ThT fluorescence curves and high pellet fractions observed for reactions without MAP proteins.

With continued reference to panel c of FIG. 15, the middle column displays images for AD samples with MAP2, and the right column displays images for AD samples with MAP4. The images in the middle and right columns show absence of fibrillar species, with the fields of view containing small amorphous particles rather than the elongated fibrillar structures visible in the left column. The absence of fibrillar species in the presence of MAP2 and MAP4 confirms the inhibitory effects observed through ThT fluorescence and sedimentation analysis, providing visual validation that both polypeptides prevent formation of Tau fibrils from AD brain-derived seeds.

The three-by-three grid organization in panel c of FIG. 15 demonstrates reproducibility of inhibitory activity across biological replicates. The consistent presence of fibrils in the left column across all three AD samples demonstrates that the AD brain-derived seeds support robust Tau fibril formation, while the consistent absence of fibrils in the middle and right columns demonstrates that both MAP2 and MAP4 exhibit reproducible inhibitory activity against Tau aggregation from multiple patient-derived seed sources.

The combination of ThT fluorescence kinetics, sedimentation analysis, and electron microscopy imaging shown in FIG. 15 provides multiple independent lines of evidence that MAP2 and MAP4 inhibit 4R Tau aggregation from AD brain-derived seeds. Since Alzheimer's disease is a mixed 3R+4R tauopathy, the demonstration that MAP2 and MAP4 inhibit 4R Tau aggregation from AD seeds, combined with inhibition of 3R Tau aggregation demonstrated in previous figures, indicates that MAP-based polypeptide constructs may exhibit activity against both Tau isoforms present in AD pathology.

The inhibition of 4R Tau aggregation from AD brain-derived seeds demonstrated in FIG. 15 provides advantages in various aspects for therapeutic applications targeting Alzheimer's disease. Since Alzheimer's disease is a mixed 3R+4R tauopathy, therapeutic agents targeting AD-associated Tau pathology may benefit from activity against both 3R and 4R Tau isoforms. The demonstration that MAP2 and MAP4 inhibit 4R Tau aggregation from AD seeds, combined with inhibition of 3R Tau aggregation demonstrated in previous figures, indicates that MAP-based polypeptide constructs may exhibit activity against both Tau isoforms present in AD pathology.

The cofactor-free inhibition approach demonstrated in FIG. 15 preserves the native structure of disease-associated fibrils extracted from clinical patient tissue. The polypeptides remain in the soluble supernatant fraction without forming amorphous aggregates during inhibition assays, in contrast to prior techniques using cofactors where inhibitor proteins appeared in the pellet fraction.

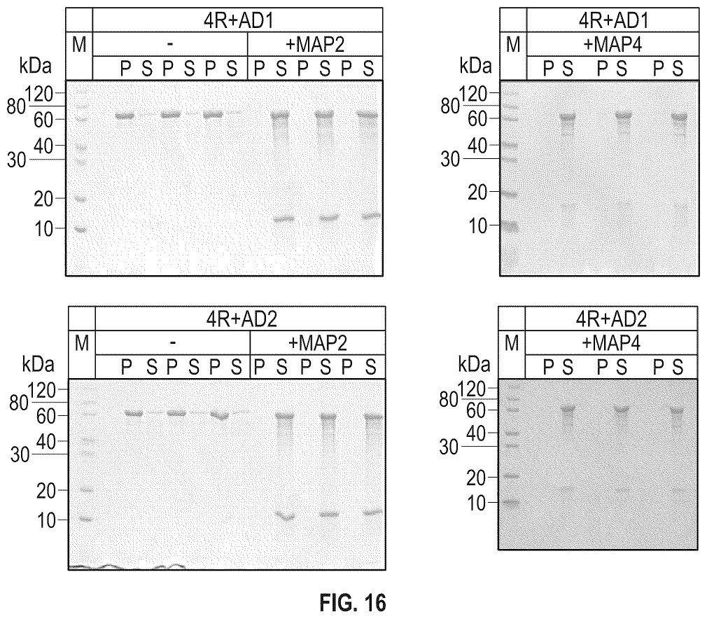

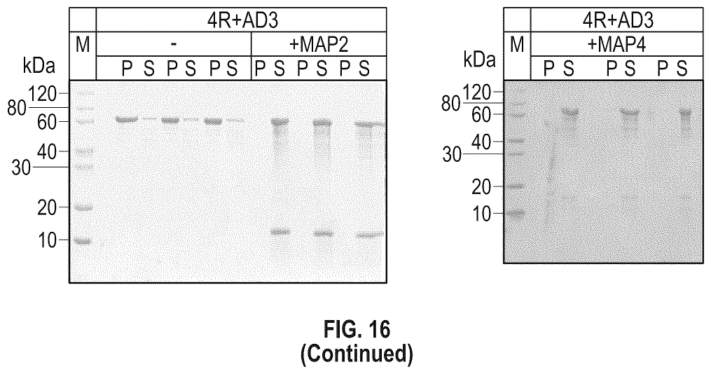

Referring to FIG. 16, SDS-PAGE gel images showing sedimentation analysis of samples after RT-QuIC assays using AD brain-derived seeds with 4R Tau substrate in the presence and absence of MAP2 and MAP4 are presented. FIG. 16 is organized into six gel panels arranged in two columns and three rows. The left column shows results for reactions with MAP2 treatment, while the right column shows results for reactions with MAP4 treatment. Each row corresponds to a different biological sample designated as 4R+AD1, 4R+AD2, and 4R+AD3, enabling systematic comparison of sedimentation behavior across three patient-derived seed sources and two polypeptide treatments.

Each gel panel shown in FIG. 16 includes a protein marker lane labeled M on the left side with molecular weight indicators at 120, 80, 60, 40, 30, 20, and 10 kDa. The lanes in each gel panel are organized to show pellet (P) and supernatant(S) fractions for multiple technical replicates, using the sedimentation assay methodology described above for FIG. 3B. The 4R Tau protein appears at approximately 40-60 kDa and the MAP2 or MAP4 proteins appear at approximately 10-15 kDa.

With continued reference to FIG. 16, the left column panels display lanes divided into sections showing reactions without MAP2 treatment indicated by a minus sign followed by lanes with MAP2 treatment indicated by +MAP2. In the sections without MAP2 treatment, the 4R Tau protein bands appear predominantly in the pellet fractions, with minimal protein visible in the supernatant fractions, indicating that Tau monomers have been recruited onto the AD brain-derived seeds and converted into insoluble fibrillar aggregates during the RT-QuIC reaction.

In the sections with MAP2 treatment shown in the left column of FIG. 16, the 4R Tau protein bands shift to appear predominantly in the supernatant fractions, with minimal protein visible in the pellet fractions. The MAP2 protein bands visible at approximately 10-15 kDa also appear in the supernatant fractions, indicating that MAP2 remains soluble without forming amorphous aggregates or co-sedimenting with fibrillar material during the inhibition assay.

As further shown in FIG. 16, the right column panels display lanes with MAP4 treatment indicated by +MAP4. Similar to the MAP2 results, the 4R Tau protein bands in the MAP4-treated reactions appear predominantly in the supernatant fractions rather than the pellet fractions. The shift of 4R Tau to the supernatant fraction when MAP4 is present demonstrates that MAP4 exhibits inhibitory activity comparable to MAP2 against Tau aggregation from AD brain-derived seeds. The MAP4 protein bands visible at lower molecular weights also appear in the supernatant fractions, indicating that MAP4 remains soluble without self-aggregating during the inhibition assay.

The SDS-PAGE gel visualization shown in FIG. 16 provides direct visualization of protein distribution, enabling assessment of band quality, consistency across replicates, and absence of artifacts. The visualization of both Tau and MAP protein bands in the same gel enables confirmation that both proteins remain in the supernatant fraction when inhibition occurs, supporting the capping mechanism. The consistent shift of 4R Tau to supernatant fractions across all three biological AD samples when MAP2 or MAP4 is present demonstrates reproducible inhibitory activity against 4R Tau aggregation from AD brain-derived seeds obtained from multiple patients. The retention of MAP2 and MAP4 in the supernatant fraction distinguishes the cofactor-free inhibition approach from prior techniques using cofactors, where inhibitor proteins appeared in the pellet fraction.

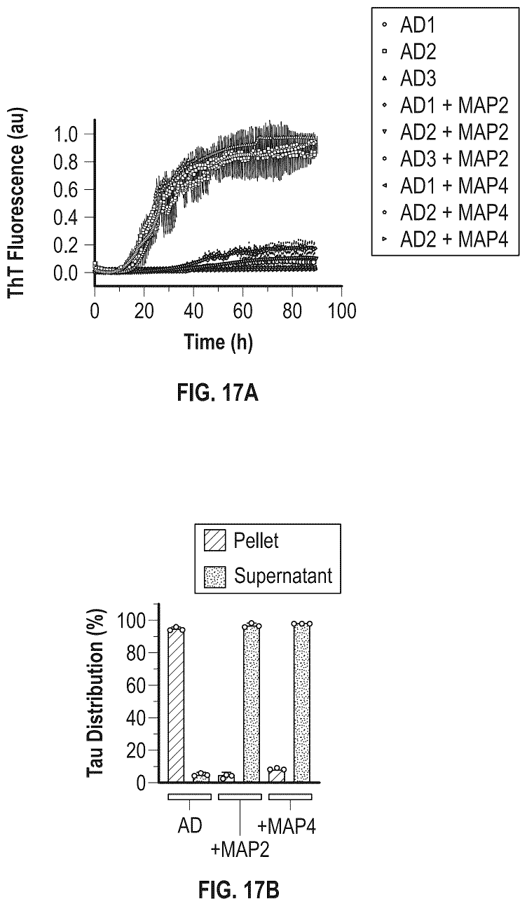

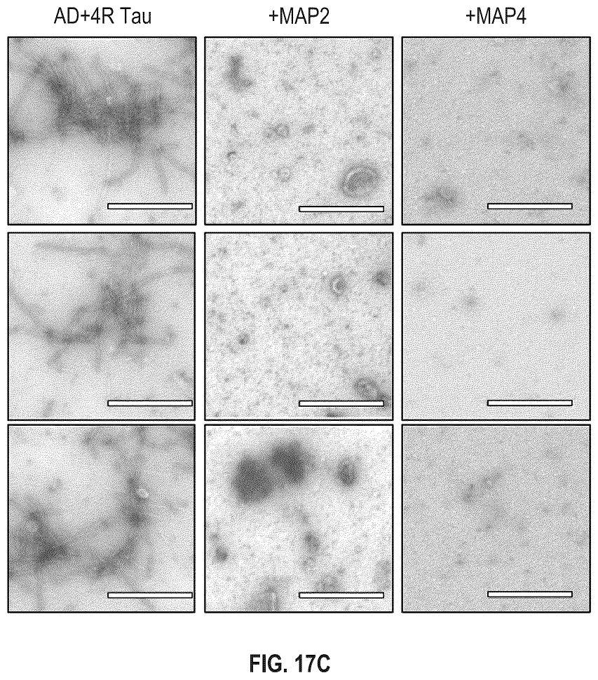

Referring to FIG. 17, a graph shows primary Thioflavin T fluorescence data from RT-QuIC reactions of tissue extracts from Alzheimer's disease brains with 3R Tau protein in the presence of MAP2 and MAP4. The graph displays ThT Fluorescence in arbitrary units on the vertical axis ranging from 0.0 to 1.0, and Time in hours on the horizontal axis ranging from 0 to 100. Three sets of data are presented corresponding to three different biological AD samples designated AD1, AD2, and AD3.

Gray traces represent reactions with AD brain extracts and 3R Tau protein alone, exhibiting sigmoidal growth curves that reach plateau fluorescence values near 1.0 arbitrary units. Red traces represent reactions in the presence of MAP2, and orange traces represent reactions in the presence of MAP4, both remaining at low fluorescence levels throughout the time course. The shaded regions around each trace indicate standard error of mean from three technical replicates of each biological replicate. The consistent suppression of ThT fluorescence across all reaction conditions containing MAP2 or MAP4 indicates robust inhibitory activity against 4R Tau fibril formation from AD brain-derived seeds.

As further shown in FIG. 17, a bar graph in the upper right portion displays Tau Distribution as a percentage, with black bars representing pellet fractions and gray bars representing supernatant fractions for AD, AD with MAP2, and AD with MAP4 conditions. In the AD alone condition, the pellet fraction approaches approximately 90 to 100 percent while the supernatant fraction remains minimal. In the AD with MAP2 and AD with MAP4 conditions, the distribution reverses such that the supernatant fraction approaches approximately 90 to 100 percent while the pellet fraction decreases to minimal levels, demonstrating that both polypeptides reduce 3R Tau aggregation by greater than 90%.

Electron microscopy images shown in the lower portion of FIG. 17 provide visual confirmation of fibril formation and inhibition. The left column displays fibrils formed in the absence of MAP proteins, with elongated fibrillar structures of varying lengths visible against the background of the carbon-coated copper grid support. The presence of fibrillar structures in the left column correlates with the sigmoidal ThT fluorescence curves and high pellet fractions observed for reactions without MAP proteins. The center column shows reduced fibril formation with MAP2, and the right column shows reduced fibril formation with MAP4. The absence or marked reduction of fibrillar species in the center and right columns confirms the inhibitory effects observed through ThT fluorescence and sedimentation analysis.

The combination of ThT fluorescence kinetics, sedimentation analysis, and electron microscopy imaging shown in FIG. 17 confirms that MAP2 and MAP4 inhibit 3R Tau aggregation from AD brain-derived seeds through multiple independent analytical methods.

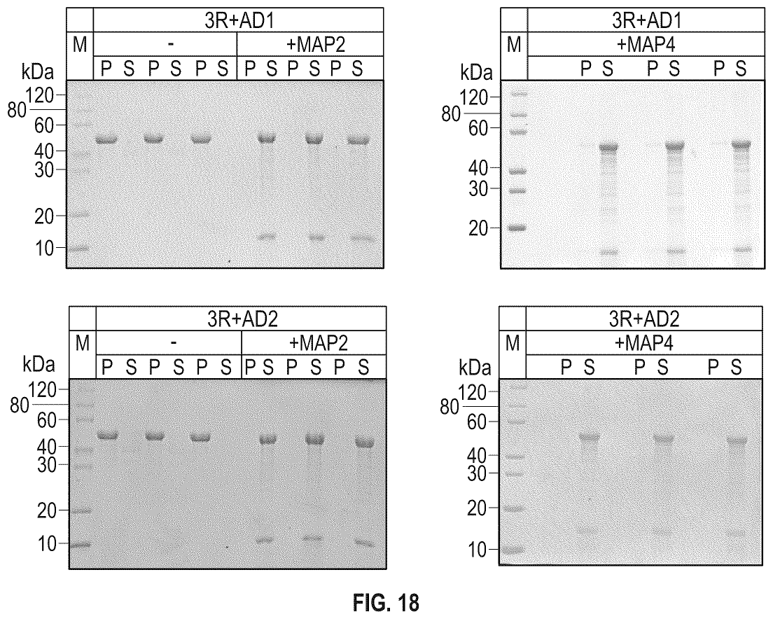

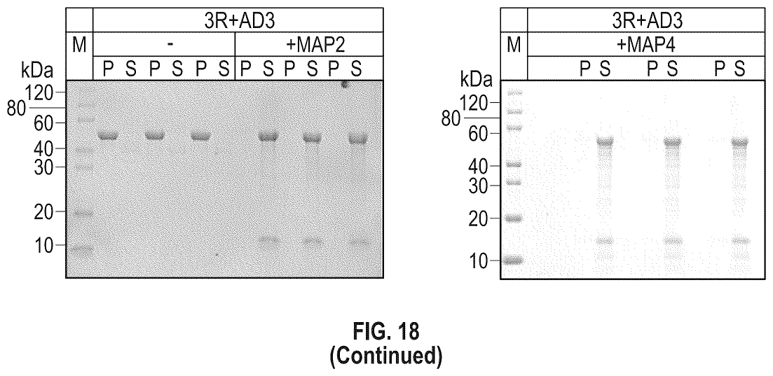

Referring to FIG. 18, SDS-PAGE gel images showing sedimentation analysis of samples after RT-QuIC assays using brain-derived seeds from three Alzheimer's disease subjects and 3R Tau protein are presented. FIG. 18 is organized into six gel panels arranged in two rows of three panels each. The top row shows results for 3R Tau with AD1, AD2, and AD3 brain samples in the presence of MAP2, while the bottom row shows results for the same brain samples in the presence of MAP4. The six-panel organization enables systematic comparison of sedimentation behavior across three patient-derived seed sources and two polypeptide treatments when 3R Tau is used as the reaction substrate.

Each gel panel shown in FIG. 18 includes a protein marker lane labeled M on the left side with molecular weight indicators at 120, 80, 60, 40, 30, 20, and 10 kDa. The lanes in each gel panel are organized to show pellet (P) and supernatant(S) fractions under different treatment conditions, using the sedimentation assay methodology described above for FIG. 3B.

With continued reference to FIG. 18, the top row panels display lanes for samples without MAP2 treatment indicated by a minus sign followed by lanes with MAP2 treatment indicated by +MAP2. In the sections without MAP2 treatment, the 3R Tau protein bands appear predominantly in the pellet fractions, indicating that 3R Tau monomers have been recruited onto the AD brain-derived seeds and converted into insoluble fibrillar aggregates, consistent with the mixed 3R+4R tauopathy nature of Alzheimer's disease.

In the sections with MAP2 treatment shown in the top row of FIG. 18, the 3R Tau protein bands shift to appear predominantly in the supernatant fractions. The MAP2 protein bands visible at lower molecular weights also appear in the supernatant fractions, indicating that MAP2 remains soluble without forming amorphous aggregates during the inhibition assay.

As further shown in FIG. 18, the bottom row panels display lanes with MAP4 treatment indicated by +MAP4. Similar to the MAP2 results observed in the top row, the 3R Tau protein bands in the MAP4-treated reactions appear predominantly in the supernatant fractions rather than the pellet fractions. The shift of 3R Tau to the supernatant fraction when MAP4 is present demonstrates that MAP4 exhibits inhibitory activity comparable to MAP2 against 3R Tau aggregation from AD brain-derived seeds. The MAP4 protein bands visible at lower molecular weights also appear in the supernatant fractions, indicating that MAP4 remains soluble without self-aggregating during the inhibition assay.

The consistent shift of 3R Tau to supernatant fractions across all three biological AD samples when MAP2 or MAP4 is present demonstrates reproducible inhibitory activity against 3R Tau aggregation from AD brain-derived seeds obtained from multiple patients. The retention of MAP2 and MAP4 in the supernatant fraction confirms that the polypeptides do not self-aggregate or form non-specific precipitates during the inhibition assay.

The retention of MAP2 and MAP4 in the supernatant fraction provides advantages in various aspects for distinguishing the cofactor-free inhibition approach from prior techniques using cofactors. The gel images in FIG. 18 demonstrate that in the cofactor-free approach, both MAP2 and MAP4 remain in the soluble supernatant fraction along with the 3R Tau protein, indicating that the polypeptides do not self-aggregate or form non-specific precipitates during the inhibition assay. The clear separation of soluble and insoluble fractions in the cofactor-free approach enables unambiguous assessment of inhibitory activity against disease-relevant fibril structures.

The inhibition of 3R Tau aggregation from AD brain-derived seeds demonstrated in FIG. 18, combined with inhibition of 4R Tau aggregation shown in previous figures, indicates that MAP-based polypeptide constructs may exhibit activity against both Tau isoforms present in AD pathology. The ability of AD brain-derived seeds to template aggregation of both 3R and 4R Tau substrates confirms the mixed tauopathy nature of AD-derived seeds.

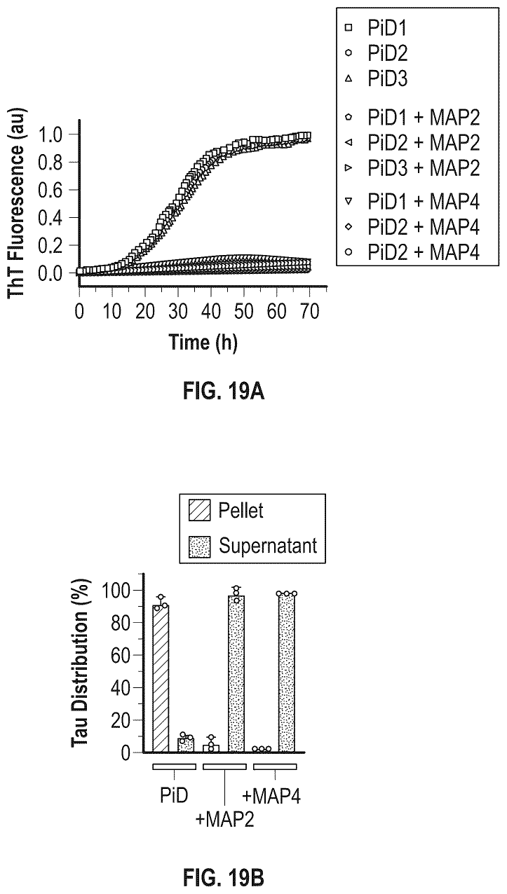

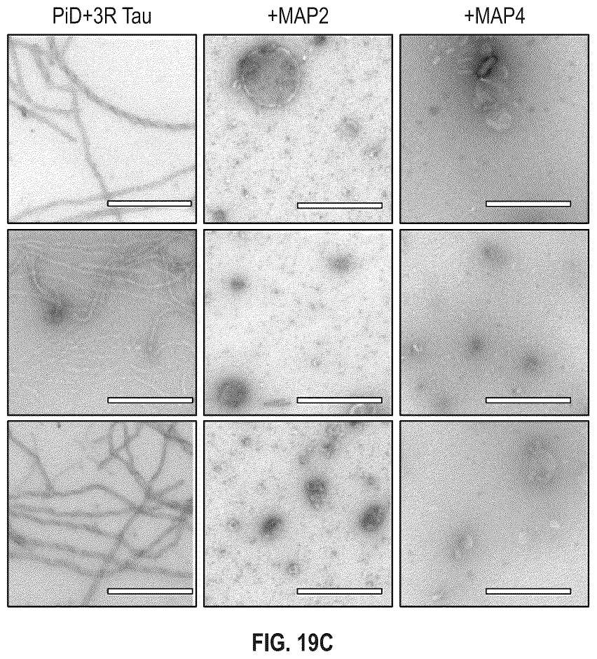

Referring to FIG. 19, a multi-panel graph shows primary Thioflavin T (ThT) fluorescence data from RT-QuIC reactions of tissue extracts from Pick's disease (PiD) brains, illustrating inhibitory behavior of MAP2 and MAP4 against 3R Tau aggregation. FIG. 19 presents data for three different biological PiD samples designated PiD1, PiD2, and PiD3. The horizontal axis represents time in hours ranging from 0 to 70 hours, while the vertical axis represents ThT fluorescence in arbitrary units ranging from 0.0 to 1.0.

Gray traces represent reactions with Pick's disease brain extracts and 3R Tau protein alone, exhibiting sigmoidal growth curves that reach fluorescence values approaching 1.0 arbitrary units. Blue traces represent reactions in the presence of MAP2 or MAP4, remaining at low fluorescence values near the baseline throughout the 70-hour time period. The polypeptide may be used at equimolar concentration with Tau substrate in inhibition assays. The reactions were carried out in triplicates from three different biological Pick's disease samples, demonstrating that both MAP2 and MAP4 inhibit 3R Tau aggregation from PiD brain-derived seeds.

The inhibition of 3R Tau aggregation from PiD brain-derived seeds demonstrated in FIG. 19 provides advantages in various embodiments for therapeutic applications targeting Pick's disease. Since Pick's disease is a 3R-exclusive tauopathy characterized by pathological deposits containing predominantly 3R Tau isoforms, the demonstration that both MAP2 and MAP4 inhibit 3R Tau aggregation from PiD seeds indicates that MAP-based polypeptide constructs may exhibit activity against the Tau species contributing to PiD-associated pathology. The cofactor-free inhibition approach preserves the native structure of disease-associated fibrils extracted from clinical patient tissue.

The broad-spectrum activity of MAP2 and MAP4 against PiD fibrils, combined with the inhibitory activity against AD and PSP fibrils demonstrated in previous figures, indicates that MAP-based polypeptide constructs may exhibit activity against diverse Tau fibril conformations characterized by different structural topologies. In some cases, the broad-spectrum capping behavior may enable development of therapeutic agents effective against multiple tauopathies without requiring disease-specific modifications to the polypeptide construct.

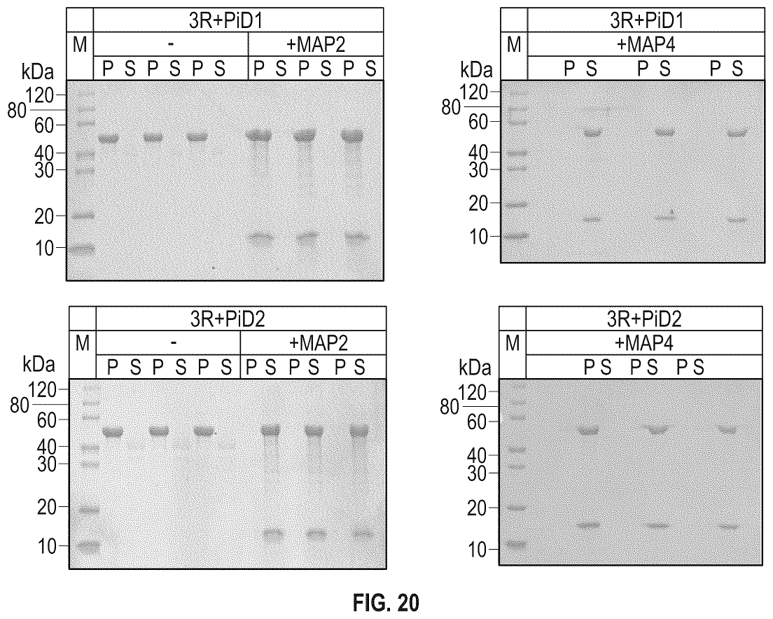

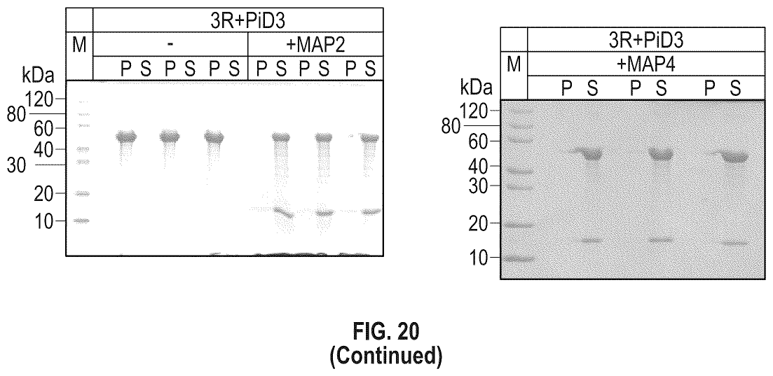

Referring to FIG. 20, SDS-PAGE gel images showing sedimentation analysis of MAP2 and MAP4 inhibition of 3R Tau aggregation from Pick's disease brain-derived samples are presented. FIG. 20 is organized into six gel panels arranged in two rows of three panels each. The top row shows results for 3R Tau with PiD1, PiD2, and PiD3 brain samples in the presence of MAP2, while the bottom row shows results for the same brain samples in the presence of MAP4. The six-panel organization enables systematic comparison of sedimentation behavior across three patient-derived seed sources and two polypeptide treatments when 3R Tau is used as the reaction substrate with PiD brain-derived seeds.

Each gel panel shown in FIG. 20 includes a protein marker lane labeled M on the left side with molecular weight indicators at 120, 80, 60, 40, 30, 20, and 10 kDa. The lanes in each gel panel are organized to show pellet (P) and supernatant(S) fractions for multiple technical replicates, using the sedimentation assay methodology described above for FIG. 3B.

With continued reference to FIG. 20, the top row panels display lanes for samples without MAP2 treatment indicated by a minus sign followed by lanes with MAP2 treatment indicated by +MAP2. In the sections without MAP2 treatment, the 3R Tau protein bands appear predominantly in the pellet fractions, indicating that 3R Tau monomers have been recruited onto the PiD brain-derived seeds and converted into insoluble fibrillar aggregates during the RT-QuIC reaction.

In the sections with MAP2 treatment shown in the top row of FIG. 20, the 3R Tau protein bands shift to appear predominantly in the supernatant fractions. The MAP2 protein bands visible at lower molecular weights also appear in the supernatant fractions, indicating that MAP2 remains soluble without forming amorphous aggregates during the inhibition assay.

As further shown in FIG. 20, the bottom row panels display lanes with MAP4 treatment indicated by +MAP4. Similar to the MAP2 results observed in the top row, the 3R Tau protein bands in the MAP4-treated reactions appear predominantly in the supernatant fractions rather than the pellet fractions. The shift of 3R Tau to the supernatant fraction when MAP4 is present demonstrates that MAP4 exhibits inhibitory activity comparable to MAP2 against 3R Tau aggregation from PiD brain-derived seeds. The MAP4 protein bands visible at lower molecular weights also appear in the supernatant fractions, indicating that MAP4 remains soluble without self-aggregating during the inhibition assay.

The consistent shift of 3R Tau to supernatant fractions across all three biological PiD samples when MAP2 or MAP4 is present demonstrates reproducible inhibitory activity against 3R Tau aggregation from PiD brain-derived seeds obtained from multiple patients. The retention of MAP2 and MAP4 in the supernatant fraction confirms that the polypeptides do not self-aggregate or form non-specific precipitates during the inhibition assay, enabling unambiguous assessment of inhibitory activity against disease-relevant PiD fibril structures.

The inhibition of 3R Tau aggregation from PiD brain-derived seeds demonstrated in FIG. 20 indicates that MAP-based polypeptide constructs may exhibit activity against the Tau species contributing to PiD-associated pathology. The ability of PiD brain-derived seeds to template aggregation of 3R Tau substrates confirms the 3R-exclusive tauopathy nature of PiD-derived seeds.

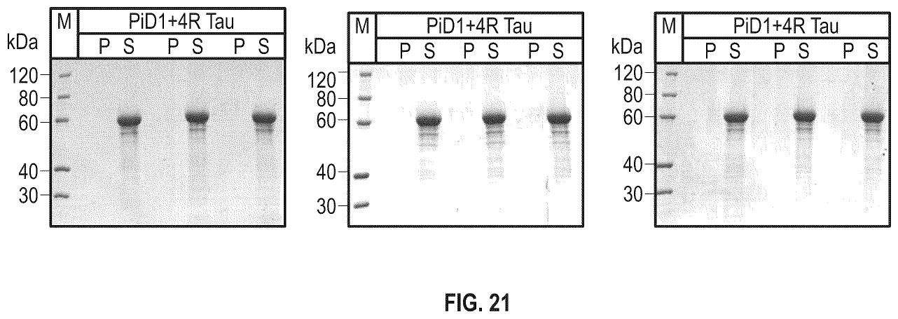

Referring to FIG. 21, sedimentation analysis validating the seeding barrier between Pick's disease brain-derived seeds and 4R Tau protein is presented. FIG. 21 displays three separate gel electrophoresis panels, each corresponding to a different biological sample from Pick's disease subjects, labeled as PiD1+4R Tau, PiD2+4R Tau, and PiD3+4R Tau. The three-panel organization enables systematic assessment of whether PiD brain-derived seeds support aggregation of 4R Tau substrate across multiple patient-derived seed sources.

Each gel panel shown in FIG. 21 includes a protein marker lane designated M on the left side with molecular weight indicators at 120, 80, 60, 40, and 30 kDa. Each panel displays three pairs of lanes marked P and S, representing pellet and supernatant fractions respectively for multiple technical replicates, using the sedimentation assay methodology described above for FIG. 3B.

With continued reference to FIG. 21, the gel images demonstrate that 4R Tau protein remains predominantly in the supernatant fractions rather than the pellet fractions across all three Pick's disease brain samples. The protein bands visible at approximately 40-60 kDa corresponding to 4R Tau appear in the supernatant lanes with minimal protein visible in the pellet lanes. The predominant presence of 4R Tau in the supernatant fractions indicates that PiD brain-derived seeds do not catalyze 4R Tau aggregation under the RT-QuIC assay conditions. The distribution of 4R Tau predominantly in the supernatant fraction contrasts with the distribution observed when 3R Tau is used as the substrate with PiD seeds, where 3R Tau appears predominantly in the pellet fraction as demonstrated in previous figures.

The sedimentation analysis shown in FIG. 21 confirms the seeding barrier characteristic of 3R-exclusive tauopathies. Pick's disease is characterized as a 3R-exclusive tauopathy, meaning that pathological deposits in PiD contain predominantly 3R Tau isoforms rather than 4R Tau isoforms. The seeding barrier refers to the inability of disease-associated fibril seeds to template aggregation of Tau isoforms that differ from the isoform composition of the original pathological deposits. The gel images in FIG. 21 demonstrate that PiD-derived seeds selectively template 3R Tau aggregation but do not template 4R Tau aggregation, consistent with the 3R-exclusive nature of Pick's disease pathology.

As further shown in FIG. 21, the consistent distribution of 4R Tau in the supernatant fraction across all three biological PiD samples and all technical replicates demonstrates reproducibility of the seeding barrier observation. The absence of 4R Tau in the pellet fractions indicates that the PiD brain-derived seeds do not provide a compatible template for 4R Tau monomer recruitment and fibril elongation.

The seeding barrier validation shown in FIG. 21 confirms the isoform specificity of disease-associated Tau seeds. The demonstration that PiD seeds do not support 4R Tau aggregation provides validation that the PiD brain homogenates retain the 3R-exclusive tauopathy nature. The molecular basis for the seeding barrier relates to structural differences between 3R and 4R Tau isoforms, where the additional microtubule binding repeat in 4R Tau may prevent adoption of the PiD fibril conformation.

The isoform specificity of disease-associated Tau seeds may enable development of diagnostic assays that distinguish between 3R-exclusive and 4R-exclusive tauopathies based on the ability of patient-derived seeds to template aggregation of specific Tau isoforms.

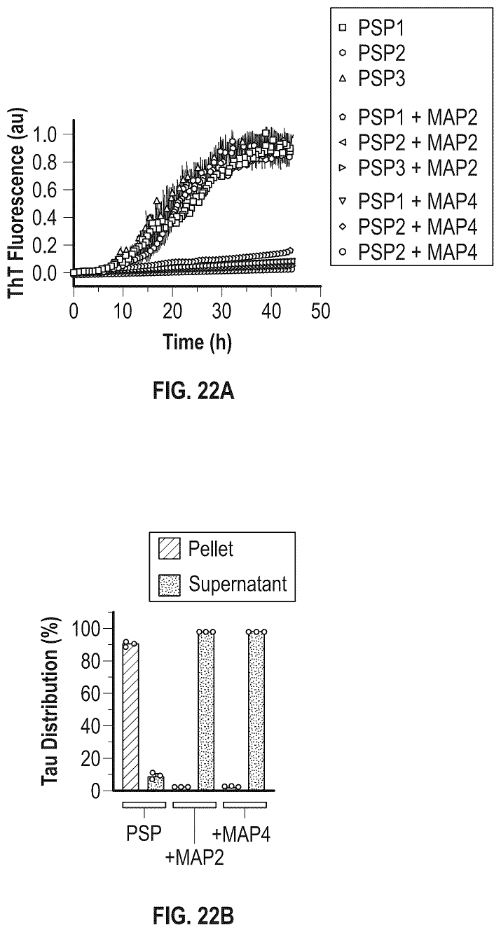

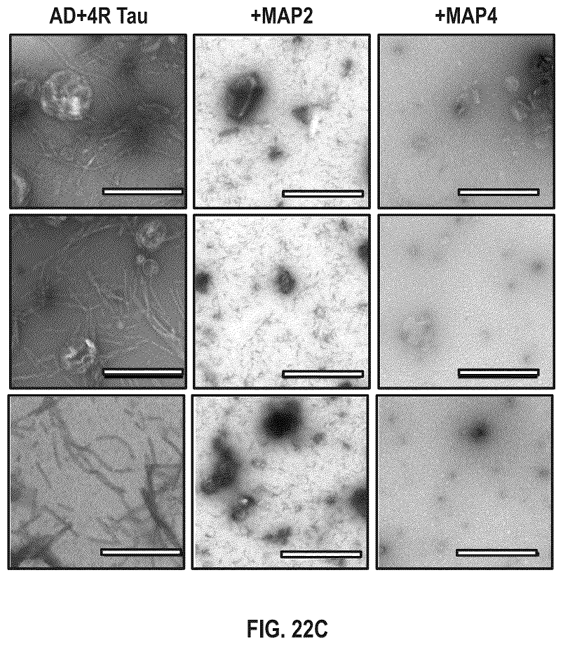

Referring to FIG. 22, a multi-panel graph shows primary Thioflavin T (ThT) fluorescence data from RT-QuIC reactions of tissue extracts from progressive supranuclear palsy (PSP) brains, illustrating inhibitory behavior of MAP2 and MAP4 against 4R Tau aggregation. FIG. 22 presents data for three different biological PSP samples designated PSP1, PSP2, and PSP3. The horizontal axis represents time in hours ranging from 0 to 50 hours, while the vertical axis represents ThT fluorescence in arbitrary units ranging from 0.0 to 1.0.

Gray traces represent reactions with PSP brain extracts and 4R Tau protein alone, exhibiting sigmoidal growth curves that reach fluorescence values approaching 1.0 arbitrary units. Green traces represent reactions in the presence of MAP2 or MAP4, remaining at low fluorescence values near the baseline throughout the 50-hour time period. The polypeptide may be used at equimolar concentration with Tau substrate in inhibition assays targeting PSP-associated Tau fibrils. The reactions were carried out in triplicates from three different biological PSP samples, with shaded regions indicating standard error of mean, demonstrating that both MAP2 and MAP4 inhibit 4R Tau aggregation from PSP brain-derived seeds.

The inhibition of 4R Tau aggregation from PSP brain-derived seeds demonstrated in FIG. 22 provides advantages in various aspects for therapeutic applications targeting progressive supranuclear palsy. Since progressive supranuclear palsy is a 4R-exclusive tauopathy characterized by pathological deposits containing predominantly 4R Tau isoforms, the demonstration that both MAP2 and MAP4 inhibit 4R Tau aggregation from PSP seeds indicates that MAP-based polypeptide constructs may exhibit activity against the Tau species contributing to PSP-associated pathology. The cofactor-free inhibition approach preserves the native structure of disease-associated fibrils extracted from clinical patient tissue.

The broad-spectrum activity of MAP2 and MAP4 against PSP fibrils, combined with the inhibitory activity against AD and PiD fibrils demonstrated in previous figures, indicates that MAP-based polypeptide constructs may exhibit activity against diverse Tau fibril conformations characterized by different structural topologies. The consistent inhibition observed across 4R-exclusive tauopathies like progressive supranuclear palsy and mixed tauopathies like Alzheimer's disease supports characterization of MAP2 and MAP4 as broad-spectrum fibril cappers suitable for development as therapeutic agents targeting Tau aggregation in diverse neurodegenerative diseases.

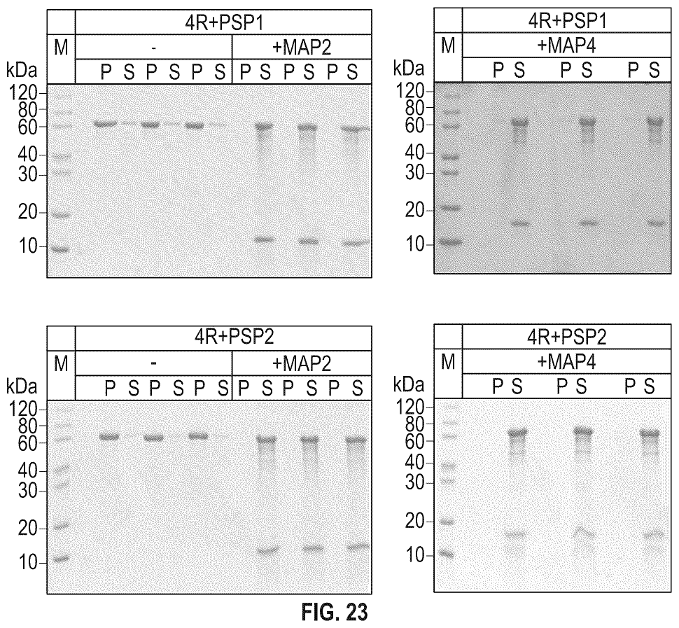

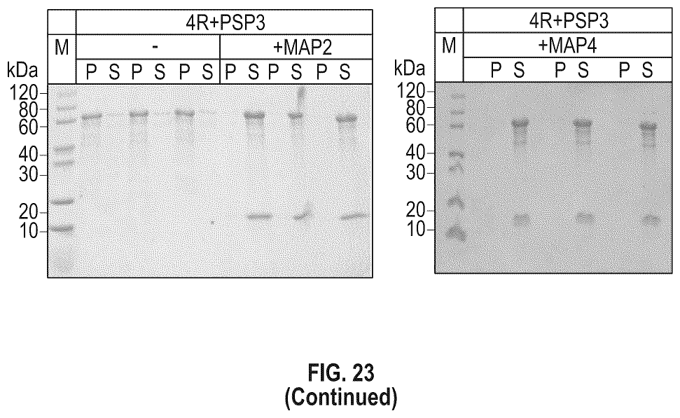

Referring to FIG. 23, SDS-PAGE gel electrophoresis results showing sedimentation analysis of samples after RT-QuIC assays using PSP brain-derived seeds and 4R Tau in the presence and absence of MAP2 and MAP4 are presented. FIG. 23 is organized into six gel panels arranged in two columns and three rows. The left column shows results for 4R Tau with PSP samples in the presence of MAP2, while the right column shows results for 4R Tau with PSP samples in the presence of MAP4. Each row corresponds to a different PSP brain sample designated as PSP1, PSP2, and PSP3, enabling systematic comparison of sedimentation behavior across three patient-derived seed sources and two polypeptide treatments.

Each gel panel shown in FIG. 23 includes a protein marker lane labeled M on the left side with molecular weight indicators at 120, 80, 60, 40, 30, 20, and 10 kDa. The lanes in each gel panel are organized to show pellet (P) and supernatant(S) fractions under different conditions, using the sedimentation assay methodology described above for FIG. 3B.

With continued reference to FIG. 23, the left column panels display lanes for samples without MAP2 treatment followed by lanes with MAP2 treatment. In the sections without MAP2 treatment, the 4R Tau protein bands appear predominantly in the pellet fractions, indicating that 4R Tau monomers have been recruited onto the PSP brain-derived seeds and converted into insoluble fibrillar aggregates during the RT-QuIC reaction.

In the sections with MAP2 treatment shown in the left column of FIG. 23, the 4R Tau protein bands shift to appear predominantly in the supernatant fractions. The MAP2 protein bands visible at lower molecular weights also appear in the supernatant fractions, indicating that MAP2 remains soluble without forming amorphous aggregates during the inhibition assay.

As further shown in FIG. 23, the right column panels display lanes with MAP4 treatment. Similar to the MAP2 results observed in the left column, the 4R Tau protein bands in the MAP4-treated reactions appear predominantly in the supernatant fractions rather than the pellet fractions. The shift of 4R Tau to the supernatant fraction when MAP4 is present demonstrates that MAP4 exhibits inhibitory activity comparable to MAP2 against 4R Tau aggregation from PSP brain-derived seeds. The MAP4 protein bands visible at lower molecular weights also appear in the supernatant fractions, indicating that MAP4 remains soluble without self-aggregating during the inhibition assay.

The consistent shift of 4R Tau to supernatant fractions across all three biological PSP samples when MAP2 or MAP4 is present demonstrates reproducible inhibitory activity against 4R Tau aggregation from PSP brain-derived seeds obtained from multiple patients. The retention of MAP2 and MAP4 in the supernatant fraction confirms that the polypeptides do not self-aggregate or form non-specific precipitates during the inhibition assay, enabling unambiguous assessment of inhibitory activity against disease-relevant PSP fibril structures.

The inhibition of 4R Tau aggregation from PSP brain-derived seeds demonstrated in FIG. 23 indicates that MAP-based polypeptide constructs may exhibit activity against the Tau species contributing to PSP-associated pathology. The ability of PSP brain-derived seeds to template aggregation of 4R Tau substrates confirms the 4R-exclusive tauopathy nature of PSP-derived seeds.

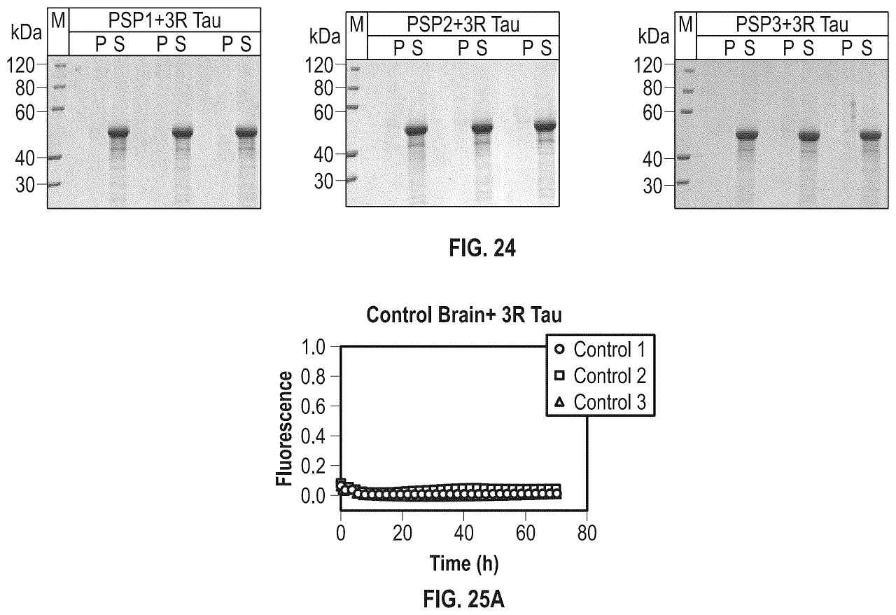

Referring to FIG. 24, a series of SDS-PAGE gel images showing sedimentation analysis of samples after RT-QuIC assays of PSP brain-derived seeds with 3R Tau is presented. FIG. 24 displays three separate gel panels, labeled PSP1+3R Tau, PSP2+3R Tau, and PSP3+3R Tau, corresponding to brain samples from three different PSP subjects. The three-panel organization enables systematic assessment of whether PSP brain-derived seeds support aggregation of 3R Tau substrate across multiple patient-derived seed sources.

Each gel panel shown in FIG. 24 includes a protein marker lane designated M with molecular weight indicators at 120, 80, 60, 40, and 30 kDa. For each PSP sample, three sets of paired lanes are shown, with each pair containing a pellet fraction designated P and a supernatant fraction designated S, using the sedimentation assay methodology described above for FIG. 3B.

With continued reference to FIG. 24, the gel images demonstrate that 3R Tau protein remains predominantly in the supernatant fractions rather than the pellet fractions across all three PSP brain samples. The protein bands appearing at approximately 40 kDa corresponding to 3R Tau are visible in the supernatant lanes with minimal protein visible in the pellet lanes, indicating that PSP brain-derived seeds do not catalyze 3R Tau aggregation under the RT-QuIC assay conditions.

The sedimentation analysis shown in FIG. 24 confirms the seeding barrier characteristic of 4R-exclusive tauopathies. Progressive supranuclear palsy is characterized as a 4R-exclusive tauopathy, meaning that pathological deposits in PSP contain predominantly 4R Tau isoforms rather than 3R Tau isoforms. The seeding barrier refers to the inability of disease-associated fibril seeds to template aggregation of Tau isoforms that differ from the isoform composition of the original pathological deposits. The gel images in FIG. 24 demonstrate that PSP-derived seeds selectively template 4R Tau aggregation but do not template 3R Tau aggregation, consistent with the 4R-exclusive nature of progressive supranuclear palsy pathology.

As further shown in FIG. 24, the consistent distribution of 3R Tau in the supernatant fraction across all three biological PSP samples and all technical replicates demonstrates reproducibility of the seeding barrier observation. The absence of 3R Tau in the pellet fractions indicates that the PSP brain-derived seeds do not provide a compatible template for 3R Tau monomer recruitment and fibril elongation.

The seeding barrier validation shown in FIG. 24 confirms the isoform specificity of PSP-associated Tau seeds. The demonstration that PSP seeds do not support 3R Tau aggregation provides validation that the PSP brain homogenates retain the 4R-exclusive tauopathy nature. The molecular basis for the seeding barrier relates to structural differences between 3R and 4R Tau isoforms, where the absence of the second microtubule binding repeat in 3R Tau may prevent adoption of the PSP fibril conformation.

The demonstration that PiD seeds template 3R Tau but not 4R Tau, combined with the demonstration that PSP seeds template 4R Tau but not 3R Tau, confirms that the disease-associated fibril seeds used in the inhibition assays retain the isoform specificity characteristic of the respective tauopathies. The preservation of isoform specificity in the cofactor-free RT-QuIC assay conditions indicates that the assay system maintains disease-relevant fibril structures suitable for evaluating polypeptide inhibitory activity against authentic disease-associated Tau conformations.

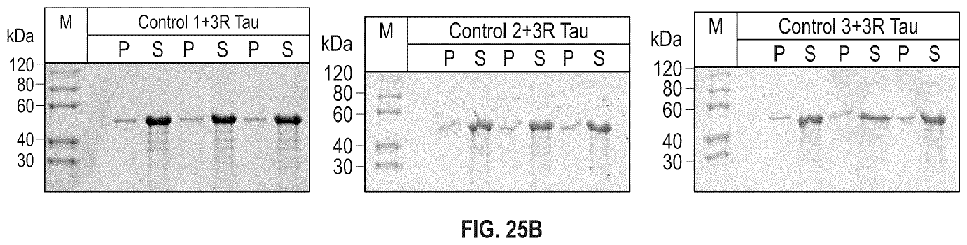

Referring to FIG. 25, RT-QuIC assays of control brain samples with 3R Tau are presented. FIG. 25 is organized into two panels designated as panel a and panel b, providing complementary analytical perspectives on the behavior of 3R Tau protein when incubated with brain homogenates from non-demented control individuals who lack histopathology of Tau aggregates.

Panel a of FIG. 25 shows primary Thioflavin T (ThT) fluorescence data from RT-QuIC reactions of tissue extracts from control brains and 3R Tau protein. The graph displays fluorescence intensity on the vertical axis ranging from 0.0 to 1.0, and time in hours on the horizontal axis ranging from 0 to 80 hours. Three control samples are represented in the graph, with Control 1 shown in blue circles, Control 2 shown in red squares, and Control 3 shown in green triangles. The three control samples correspond to brain homogenates prepared from non-demented individuals without histopathological evidence of Tau aggregates.

With continued reference to panel a of FIG. 25, the fluorescence traces for all three control samples remain at baseline levels near 0.0 throughout the entire 80-hour time period. The absence of sigmoidal growth curves in the control sample traces contrasts with the sigmoidal fluorescence profiles observed when disease brain-derived seeds from AD, PiD, or PSP patients are used in the RT-QuIC assays. The baseline fluorescence maintained throughout the 80-hour measurement period indicates that 3R Tau monomers do not undergo spontaneous aggregation or seed-templated aggregation when incubated with control brain homogenates under the RT-QuIC assay conditions.

The reactions shown in panel a of FIG. 25 were carried out in triplicates from three different biological control subjects, providing both technical and biological replication to assess reproducibility of the absence of aggregation. The consistent baseline fluorescence observed across all nine reaction conditions demonstrates that the lack of Tau aggregation with control brain samples is reproducible across multiple patient sources and technical replicates. The absence of ThT fluorescence increase indicates that control brain homogenates do not contain proteopathic seeds capable of templating 3R Tau fibril formation.

As further shown in FIG. 25, panel b presents sedimentation analysis of samples after RT-QuIC assays by SDS-PAGE and Coomassie staining. Three separate gel images are displayed for Control 1+3R Tau, Control 2+3R Tau, and Control 3+3R Tau, corresponding to the three biological control samples examined in panel a. Each gel includes a protein marker lane labeled M on the left side with molecular weight indicators at 120, 80, 60, 40, and 30 kDa. The molecular weight markers enable identification of protein bands based on their electrophoretic mobility, with the 3R Tau protein appearing at approximately 40-60 kDa.

For each control sample shown in panel b of FIG. 25, three sets of pellet (P) and supernatant(S) fractions are displayed, representing technical replicates subjected to ultracentrifugation at 260,000 g for 30 minutes. The pellet fractions contain insoluble fibrillar aggregates that sediment during ultracentrifugation, while the supernatant fractions contain soluble monomeric protein that remains in solution. The gel images demonstrate that 3R Tau protein remains predominantly in the supernatant fractions, with bands visible around 40-60 kDa in the supernatant lanes and minimal protein visible in the pellet lanes.

The predominant presence of 3R Tau in the supernatant fractions shown in panel b of FIG. 25 indicates that the protein did not aggregate into insoluble pellet fractions when incubated with control brain samples. The distribution of 3R Tau predominantly in the supernatant fraction correlates with the baseline ThT fluorescence observed in panel a, providing independent confirmation through sedimentation analysis that 3R Tau does not undergo aggregation when incubated with control brain homogenates. The technical replicates were combined and ultracentrifuged at 260,000 g for 30 minutes prior to analysis by SDS-PAGE with Coomassie staining.

The absence of aggregation with control brain samples demonstrated in FIG. 25 validates the specificity of the RT-QuIC assay system. The control experiments rule out the possibility of external factors non-specifically catalyzing Tau polymerization under the given assay conditions, other than disease-associated fibrils or seeds. The absence of Tau aggregation with control brain homogenates confirms that the sigmoidal ThT fluorescence curves and high pellet fractions observed with disease brain-derived seeds represent specific seed-templated aggregation rather than spontaneous aggregation or non-specific effects of brain homogenate components.

The control experiments confirm that the baseline ThT fluorescence and supernatant distribution observed in MAP2 and MAP4-treated reactions with disease brain-derived seeds represents specific inhibition of seed-templated aggregation rather than non-specific suppression of Tau aggregation behavior.

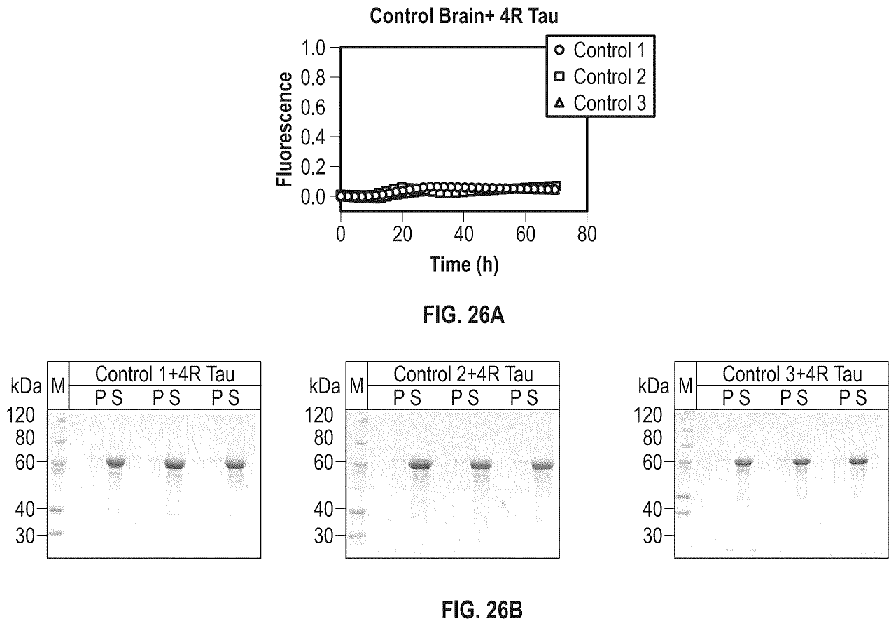

Referring to FIG. 26, RT-QuIC assays of control brain samples with 4R Tau are presented. FIG. 26 is organized into two panels designated as panel a and panel b, providing complementary analytical perspectives on the behavior of 4R Tau protein when incubated with brain homogenates from non-demented control individuals who lack histopathology of Tau aggregates.

Panel a of FIG. 26 shows a graph of primary ThT fluorescence data from RT-QuIC reactions of tissue extracts from control brains and 4R Tau protein. The graph displays fluorescence on the vertical axis ranging from 0.0 to 1.0, and time in hours on the horizontal axis ranging from 0 to 80 hours. Three control samples are represented in the graph, with Control 1 shown in blue, Control 2 shown in red, and Control 3 shown in green. The three control samples correspond to brain homogenates prepared from non-demented individuals without histopathological evidence of Tau aggregates.

With continued reference to panel a of FIG. 26, the traces for all three control samples remain near the baseline throughout the duration of the experiment. The absence of sigmoidal growth curves in the control sample traces contrasts with the sigmoidal fluorescence profiles observed when disease brain-derived seeds from AD, PiD, or PSP patients are used in the RT-QuIC assays. The baseline fluorescence maintained throughout the 80-hour measurement period indicates that 4R Tau monomers do not undergo spontaneous aggregation or seed-templated aggregation when incubated with control brain homogenates under the RT-QuIC assay conditions.

The reactions shown in panel a of FIG. 26 were carried out in triplicates from three different biological control subjects, providing both technical and biological replication to assess reproducibility of the absence of aggregation. The consistent baseline fluorescence observed across all reaction conditions demonstrates that the lack of 4R Tau aggregation with control brain samples is reproducible across multiple patient sources and technical replicates. The absence of ThT fluorescence increase indicates that control brain homogenates do not contain proteopathic seeds capable of templating 4R Tau fibril formation.

As further shown in FIG. 26, panel b presents sedimentation analysis of samples after RT-QuIC assays by SDS-PAGE and Coomassie staining. Three separate gel images are displayed for Control 1+4R Tau, Control 2+4R Tau, and Control 3+4R Tau, corresponding to the three biological control samples examined in panel a. Each gel includes a protein marker lane labeled M on the left side with molecular weight indicators at 120, 80, 60, 40, and 30 kDa. The molecular weight markers enable identification of protein bands based on their electrophoretic mobility, with the 4R Tau protein appearing at approximately 60 kDa.

For each control sample shown in panel b of FIG. 26, pellet fractions labeled P and supernatant fractions labeled S are displayed in triplicate, representing technical replicates subjected to ultracentrifugation at 260,000 g for 30 minutes. The pellet fractions contain insoluble fibrillar aggregates that sediment during ultracentrifugation, while the supernatant fractions contain soluble monomeric protein that remains in solution. The gel images demonstrate that 4R Tau protein appears predominantly in the supernatant fractions at approximately 60 kDa, with minimal protein visible in the pellet lanes.

The predominant presence of 4R Tau in the supernatant fractions shown in panel b of FIG. 26 indicates that 4R Tau remains largely in the soluble fraction when incubated with control brain samples lacking pathological Tau seeds. The distribution of 4R Tau predominantly in the supernatant fraction correlates with the baseline ThT fluorescence observed in panel a, providing independent confirmation through sedimentation analysis that 4R Tau does not undergo aggregation when incubated with control brain homogenates. The technical replicates were combined and ultracentrifuged at 260,000 g for 30 minutes prior to analysis by SDS-PAGE with Coomassie staining.

The absence of aggregation with control brain samples demonstrated in FIG. 26 validates the specificity of the RT-QuIC assay system when 4R Tau is used as the substrate. The control experiments confirm that the sigmoidal ThT fluorescence curves and high pellet fractions observed with disease brain-derived seeds represent specific seed-templated aggregation rather than spontaneous aggregation.

The combination of control experiments with 3R Tau and 4R Tau substrates provides comprehensive validation of the RT-QuIC assay system. The demonstration that control brain homogenates do not support aggregation of either 3R Tau or 4R Tau indicates that the assay system maintains specificity for disease-associated seeds regardless of which Tau isoform is used as the substrate.

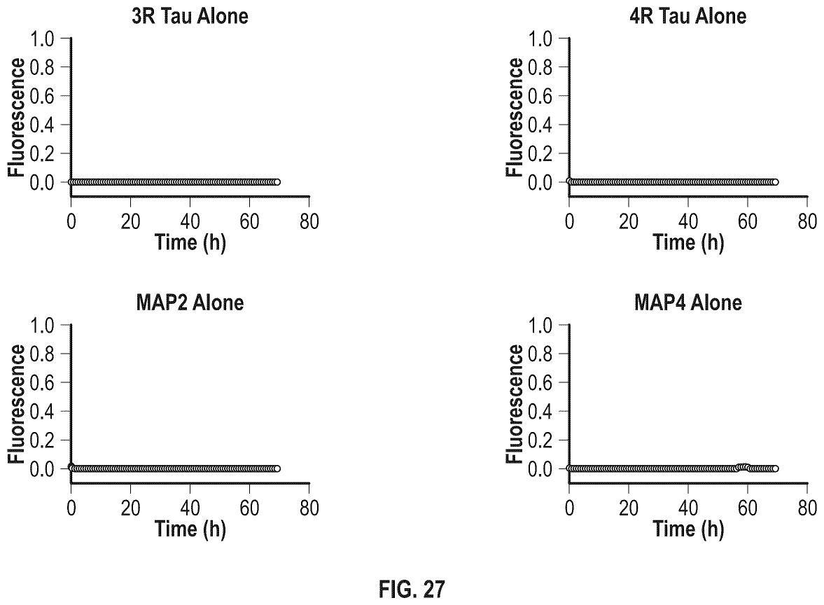

The top left graph in FIG. 27 shows 3R Tau alone with fluorescence remaining at approximately 0.0 throughout the 80-hour time period. The baseline fluorescence maintained throughout the measurement period indicates that 3R Tau monomers do not undergo spontaneous aggregation in sodium phosphate buffer without disease-associated seeds. The absence of sigmoidal growth curves in the 3R Tau alone condition contrasts with the sigmoidal fluorescence profiles observed when 3R Tau is incubated with disease brain-derived seeds from PiD or AD patients, confirming that seed-templated aggregation rather than spontaneous aggregation accounts for the fibril formation observed in disease sample reactions.

With continued reference to FIG. 27, the top right graph shows 4R Tau alone with fluorescence similarly remaining at approximately 0.0 over the same 80-hour time period. The baseline fluorescence observed for 4R Tau alone indicates that 4R Tau monomers do not undergo spontaneous aggregation under the RT-QuIC assay conditions in the absence of proteopathic seeds. The absence of spontaneous 4R Tau aggregation confirms that the sigmoidal ThT fluorescence curves observed when 4R Tau is incubated with disease brain-derived seeds from AD or PSP patients represent specific seed-templated aggregation rather than spontaneous polymerization of the Tau substrate.

The bottom left graph in FIG. 27 shows MAP2 alone with fluorescence staying at approximately 0.0 across the entire 80-hour measurement period. The baseline fluorescence maintained for MAP2 alone indicates that MAP2 does not undergo spontaneous aggregation or form ThT-positive fibrillar species under the RT-QuIC assay conditions. The absence of MAP2 self-aggregation confirms that MAP2 remains in a soluble, non-fibrillar state throughout the assay period, consistent with the sedimentation analysis results showing MAP2 predominantly in the supernatant fraction in inhibition assays.

As further shown in FIG. 27, the bottom right graph shows MAP4 alone with fluorescence remaining at approximately 0.0 throughout the time course, with a slight increase visible near the 60-hour mark. The baseline fluorescence observed for MAP4 alone indicates that MAP4 does not undergo substantial spontaneous aggregation under the RT-QuIC assay conditions. The slight fluorescence increase near the 60-hour mark remains minimal compared to the sigmoidal growth curves observed in disease sample reactions, indicating that any minor fluorescence change does not represent formation of substantial fibrillar species.

The absence of any substantial change in ThT fluorescence intensity over time in all four graphs shown in FIG. 27 indicates the absence of spontaneous aggregation for 3R Tau, 4R Tau, MAP2, and MAP4 when incubated alone in sodium phosphate buffer without disease-associated seeds. The spontaneous aggregation assays confirm that the proteins used in the inhibition assays do not undergo self-assembly into ThT-positive fibrillar species under the assay conditions, ruling out the possibility that spontaneous aggregation of substrate proteins may confound interpretation of seed-templated aggregation or inhibition results.

The spontaneous aggregation assays shown in FIG. 27 validate the specificity of the RT-QuIC assay system. The demonstration that neither Tau substrates nor MAP proteins undergo spontaneous aggregation confirms that the sigmoidal ThT fluorescence curves observed in disease sample reactions represent specific seed-templated aggregation rather than spontaneous polymerization. The absence of MAP2 and MAP4 self-aggregation confirms that the baseline ThT fluorescence observed in MAP-treated disease sample reactions represents inhibition of seed-templated Tau aggregation rather than competition between Tau aggregation and MAP aggregation, supporting the capping mechanism.

The absence of spontaneous Tau aggregation in the cofactor-free buffer system indicates that the assay conditions do not artificially promote Tau polymerization in the absence of disease-associated seeds, in contrast to prior techniques using cofactors such as heparin where spontaneous Tau aggregation may occur.

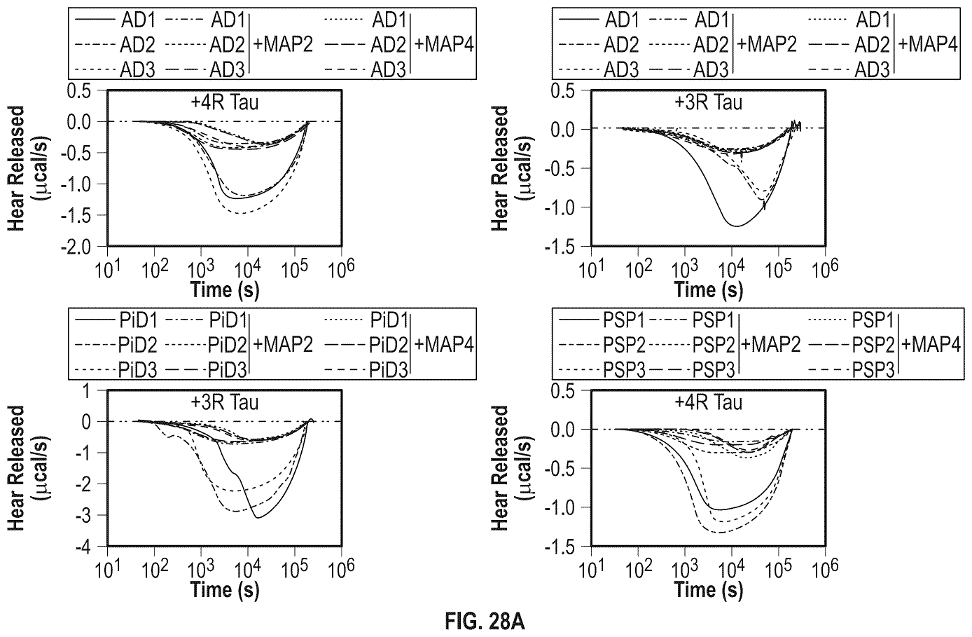

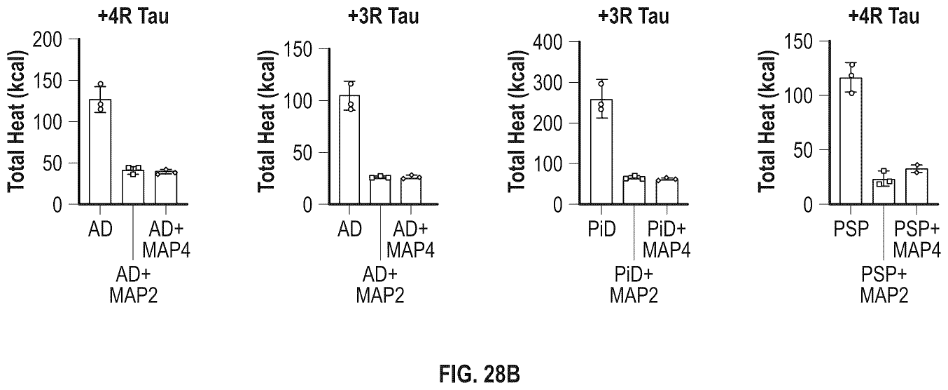

Referring to FIG. 28, isothermal titration calorimetry (ITC) data showing heat signatures of 3R and 4R Tau seeding from brain homogenates in the absence and presence of MAP2 and MAP4 is presented. FIG. 28 is organized into two panels designated as panel a and panel b, providing complementary analytical perspectives on the thermodynamic changes that accompany Tau aggregation from disease brain-derived seeds and the modulation of thermodynamic signatures by polypeptide cappers.

Panel a of FIG. 28 shows kinetics of fibril elongation from seeds derived from AD, PiD, and PSP samples, with heat released measured in microcal per second plotted against time in seconds on a logarithmic scale. The panel contains four separate graphs corresponding to different disease and substrate combinations. The upper left graph shows AD samples with 4R Tau, displaying curves for AD1, AD2, and AD3 alone and in the presence of MAP2 and MAP4. The upper right graph shows AD samples with 3R Tau under the same conditions. The lower left graph shows PiD samples with 3R Tau, displaying curves for PiD1, PiD2, and PiD3 alone and in the presence of MAP2 and MAP4. The lower right graph shows PSP samples with 4R Tau, displaying curves for PSP1, PSP2, and PSP3 alone and in the presence of MAP2 and MAP4.

With continued reference to panel a of FIG. 28, the reactions without MAP proteins show broad exothermic curves characterized by heat release over the measurement period. The thermodynamic curves for brain-seeded reactions in the absence of MAP proteins display a brief lag phase followed by heat release that peaks between negative one to negative three microcal per second. The exothermic nature of the heat release indicates that Tau fibril elongation from disease brain-derived seeds is a thermodynamically favorable process that releases energy as Tau monomers are recruited onto fibril ends and incorporated into the growing fibril structure.

The reactions in the presence of MAP2 and MAP4 shown in panel a of FIG. 28 exhibit extended lag phases with substantially lower peak heat release compared to reactions without MAP proteins. The peak heat release in MAP-treated reactions ranges from approximately negative 0.3 to negative 0.5 microcal per second, representing a reduction of approximately three-fold or greater compared to the peak heat release observed in reactions without MAP proteins. The extended lag phase and reduced peak heat release in MAP-treated reactions indicate that MAP2 and MAP4 interfere with the thermodynamic processes associated with Tau fibril elongation from disease brain-derived seeds.

As further shown in FIG. 28, panel b presents bar graphs of total heat in kcal for each experimental condition. The bar graphs enable quantitative comparison of the total enthalpy released during Tau aggregation reactions in the absence and presence of MAP proteins across the different disease types and Tau isoform substrates. The total enthalpy values were calculated by integrating the area under the ITC curves over the entire measurement period, providing a cumulative measure of the heat released during the aggregation process.

The first bar graph in panel b of FIG. 28 shows 4R Tau with AD samples, comparing AD alone, AD plus MAP2, and AD plus MAP4 conditions. The total enthalpy for AD reactions with 4R Tau alone was approximately negative 127.2 plus or minus 16.2 kcal/mol. The total enthalpy values for AD reactions in the presence of MAP2 and MAP4 were substantially reduced compared to the AD alone condition, with the reduction being approximately three-fold or greater. The second bar graph shows 3R Tau with AD samples under the same comparisons, with the total enthalpy for AD reactions with 3R Tau alone being approximately negative 105.1 plus or minus 18.5 kcal/mol. The total enthalpy for 4R Tau with AD was slightly higher than that observed for 3R Tau with AD, indicating comparable but distinct thermodynamic signatures for the two Tau isoforms when seeded by AD brain-derived material.

With continued reference to panel b of FIG. 28, the third bar graph shows 3R Tau with PiD samples, comparing PiD alone, PiD plus MAP2, and PiD plus MAP4 conditions. The total enthalpy for PiD reactions with 3R Tau alone was approximately negative 259.1 plus or minus 47.4 kcal/mol, representing the highest total heat release among the disease types examined. The fourth bar graph shows 4R Tau with PSP samples, comparing PSP alone, PSP plus MAP2, and PSP plus MAP4 conditions. The total enthalpy for PSP reactions with 4R Tau alone was approximately negative 116.5 plus or minus 13.2 kcal/mol, which was comparable to the total enthalpy observed for 4R Tau with AD but significantly lower than the total enthalpy for 3R Tau with PiD. Error bars on each measurement represent standard error of mean calculated from three biological replicates in the respective disease type.

The total heat released in the presence of MAP2 and MAP4 was observed to be approximately three-fold lower than the total heat release for AD, PiD, and PSP brain-seeded reactions without MAP proteins. The reduced total enthalpy in MAP-treated reactions indicates that MAP2 and MAP4 prevent the thermodynamic processes associated with Tau fibril formation from different brain-derived seeds. The consistent reduction in total enthalpy across all disease types and Tau isoform substrates examined demonstrates that the thermodynamic inhibition by MAP proteins operates across diverse tauopathy-associated fibril conformations.

The polypeptide may be used in isothermal titration calorimetry assays to monitor thermodynamic changes during Tau aggregation inhibition. ITC provides a sensitive technique for assessing thermodynamic changes during Tau aggregation that complements the kinetic information obtained from ThT fluorescence assays. The ITC approach measures heat changes directly, enabling detection of aggregation processes that may not be readily detected through dye binding or sedimentation methods. The kinetic ITC experiments were carried out by single injection of Tau monomers into the cell containing seeds from disease brain samples, with resulting heat changes measured over time.

The polypeptide may be used in isothermal titration calorimetry assays to monitor thermodynamic changes during Tau aggregation inhibition. ITC provides a sensitive technique for assessing thermodynamic changes during Tau aggregation that complements the kinetic information obtained from ThT fluorescence assays. The ITC approach measures heat changes directly, enabling detection of aggregation processes that may not be readily detected through dye binding or sedimentation methods.

The thermodynamic evidence for inhibition shown in FIG. 28 indicates that MAP proteins delay the onset of thermodynamically detectable aggregation processes, consistent with capping of fibril ends that reduces the number of available sites for Tau monomer recruitment. The approximately three-fold reduction in total enthalpy observed across all disease types indicates robust inhibitory activity that substantially interferes with the thermodynamic processes driving Tau fibril elongation.

Separate equilibrium ITC experiments involving titration of MAP proteins with Tau monomers yielded negligible heat changes, indicating absence of physiological interactions between Tau monomers and MAP proteins. This indicates that the inhibitory potential of MAP proteins may be attributed to binding and capping of fibril ends rather than sequestration or conformational modulation of the Tau monomer substrate.

The consistent thermodynamic inhibition observed across AD, PiD, and PSP brain-derived seeds demonstrates broad-spectrum activity of MAP proteins against diverse tauopathy-associated fibril conformations.

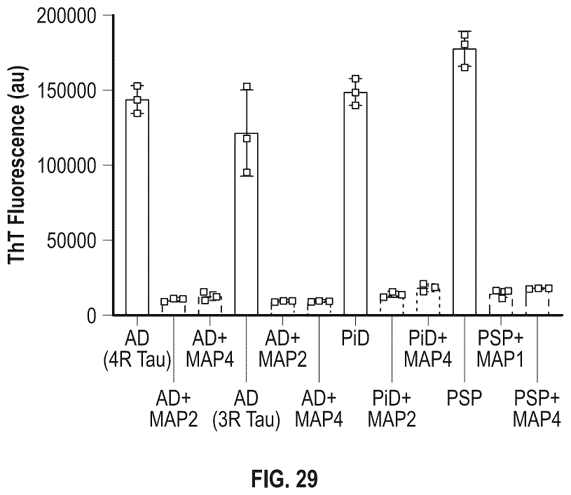

Referring to FIG. 29, a bar graph showing Thioflavin T (ThT) fluorescence measurements in arbitrary units for various experimental conditions involving tissue extracts from Alzheimer's disease (AD), Pick's disease (PiD), and progressive supranuclear palsy (PSP) brains is presented. FIG. 29 displays ThT fluorescence values on the vertical axis ranging from 0 to 200,000 arbitrary units, with the horizontal axis showing different experimental conditions for each disease type in the absence and presence of MAP2 and MAP4. The ThT fluorescence measurements were obtained from analysis of ITC reaction end-products, providing an independent confirmation of aggregation status following the thermodynamic measurements.

The AD (4R Tau) condition shown in FIG. 29 exhibits a fluorescence value of approximately 145,000 arbitrary units, indicating formation of ThT-positive fibrillar species during the ITC reaction when 4R Tau is incubated with AD brain-derived seeds in the absence of MAP proteins. The corresponding AD+MAP2 and AD+MAP4 conditions display substantially reduced fluorescence values near baseline levels, indicating that both MAP2 and MAP4 prevent formation of ThT-positive fibrillar species from AD brain-derived seeds when 4R Tau is used as the substrate. The reduction from approximately 145,000 arbitrary units to near baseline levels represents greater than 90% inhibition of fibril formation as measured by ThT fluorescence of ITC endpoint products.

With continued reference to FIG. 29, the AD (3R Tau) condition exhibits a fluorescence value of approximately 120,000 arbitrary units, indicating that AD brain-derived seeds also support formation of ThT-positive fibrillar species when 3R Tau is used as the substrate. The corresponding AD+MAP2 and AD+MAP4 conditions again show markedly reduced fluorescence near baseline levels, demonstrating that both MAP proteins inhibit 3R Tau aggregation from AD brain-derived seeds. The inhibition of both 3R and 4R Tau aggregation from AD seeds confirms the broad activity of MAP proteins against the mixed 3R+4R tauopathy nature of Alzheimer's disease.

The PiD condition shown in FIG. 29 demonstrates a fluorescence value of approximately 150,000 arbitrary units, indicating formation of ThT-positive fibrillar species when 3R Tau is incubated with PiD brain-derived seeds in the absence of MAP proteins. The PiD+MAP2 and PiD+MAP4 conditions show reduced fluorescence values near baseline levels, demonstrating that both MAP2 and MAP4 inhibit 3R Tau aggregation from PiD brain-derived seeds. The reduction from approximately 150,000 arbitrary units to near baseline levels in the presence of MAP proteins confirms the inhibitory activity observed through the thermodynamic measurements in the ITC assays.

As further shown in FIG. 29, the PSP condition displays the highest fluorescence value of approximately 180,000 arbitrary units among the disease types examined, indicating robust formation of ThT-positive fibrillar species when 4R Tau is incubated with PSP brain-derived seeds in the absence of MAP proteins. The PSP+MAP2 and PSP+MAP4 conditions exhibit substantially reduced fluorescence values near baseline levels, demonstrating that both MAP2 and MAP4 inhibit 4R Tau aggregation from PSP brain-derived seeds. Error bars are present on each bar indicating variability in the measurements across the biological replicates examined.

The ThT fluorescence analysis of ITC endpoint products shown in FIG. 29 confirms that the reduced thermodynamic signatures observed in MAP-treated reactions correlate with absence of fibrillar species. The correlation between reduced total enthalpy in ITC measurements and reduced ThT fluorescence in endpoint analysis confirms that MAP2 and MAP4 prevent formation of fibrillar Tau aggregates from brain-derived seeds across all three tauopathies examined.

The consistent inhibition observed across AD, PiD, and PSP conditions demonstrates broad-spectrum activity of MAP proteins against diverse tauopathy-associated fibril conformations. The reduction from high fluorescence values in disease sample reactions to baseline levels in MAP-treated reactions represents greater than 90% inhibition of fibril formation across all disease types and Tau isoform substrates examined.

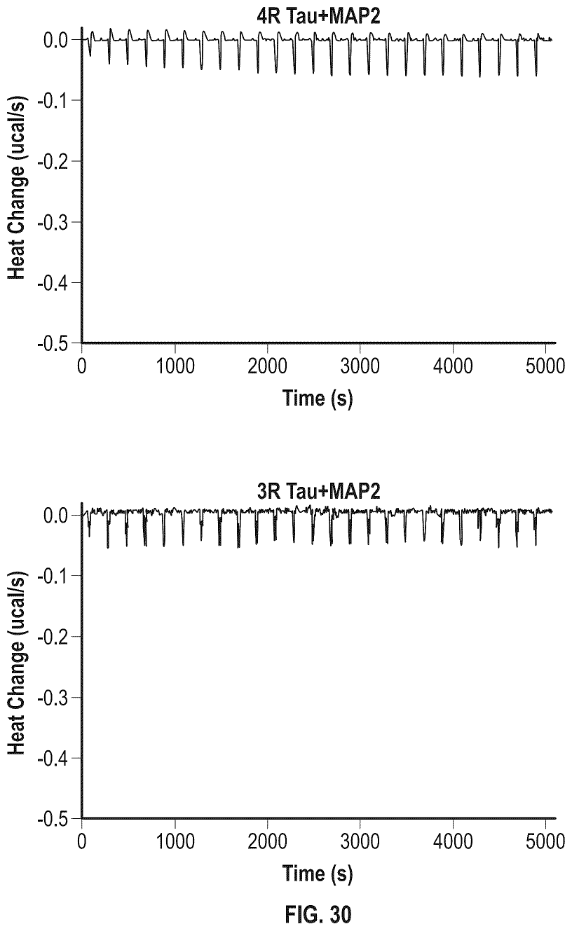

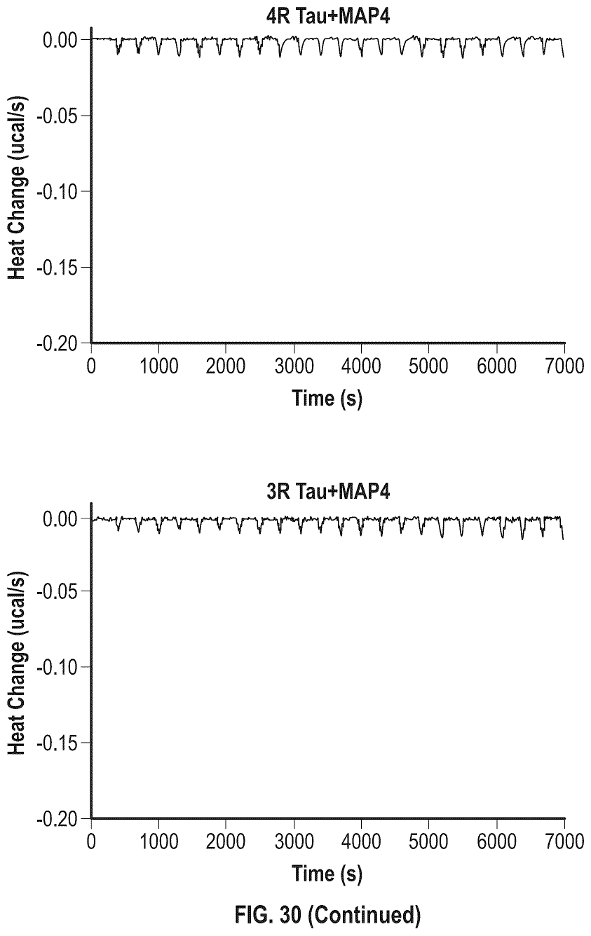

Referring to FIG. 30, four graphs showing isothermal titration calorimetry data for MAP2 and MAP4 interactions with 3R and 4R Tau monomers are presented. Each graph displays heat change in microcalories per second on the vertical axis plotted against time in seconds on the horizontal axis, enabling assessment of whether direct interactions occur between MAP proteins and Tau monomers in solution. The four-graph organization enables systematic evaluation of potential interactions between both MAP proteins and both Tau isoforms, providing comprehensive assessment of whether MAP proteins may sequester or bind to Tau monomers as a mechanism of inhibitory activity.

The top left graph in FIG. 30 shows heat change versus time for 4R Tau combined with MAP2, with the vertical axis ranging from 0.00 to −0.20 microcalories per second and the horizontal axis ranging from 0 to 7000 seconds. The graph displays continuous titration data with small periodic peaks near the baseline, indicating negligible heat changes during the titrations of MAP2 into 4R Tau monomer solution. The small periodic peaks visible in the trace correspond to the injection events during the titration, with the minimal magnitude of the heat changes indicating absence of substantial binding interactions between MAP2 and 4R Tau monomers.

With continued reference to FIG. 30, the top right graph shows heat change versus time for 3R Tau combined with MAP2, with the vertical axis ranging from 0.0 to −0.5 microcalories per second and the horizontal axis ranging from 0 to 5000 seconds. The graph displays continuous titration data with small periodic peaks near the baseline, similar to the pattern observed for 4R Tau with MAP2. The negligible heat changes observed during titration of MAP2 into 3R Tau monomer solution indicate absence of substantial binding interactions between MAP2 and 3R Tau monomers. The minimal heat release during the titration contrasts with the substantial exothermic heat release observed when Tau monomers are added to disease brain-derived seeds in the presence of MAP proteins.

The bottom left graph in FIG. 30 shows heat change versus time for 4R Tau combined with MAP4, with the vertical axis ranging from 0.00 to −0.20 microcalories per second and the horizontal axis ranging from 0 to 7000 seconds. The graph displays continuous titration data with small periodic peaks near the baseline, indicating negligible heat changes during the titrations of MAP4 into 4R Tau monomer solution. The minimal magnitude of the heat changes indicates absence of substantial binding interactions between MAP4 and 4R Tau monomers, consistent with the pattern observed for MAP2 interactions with Tau monomers.

As further shown in FIG. 30, the bottom right graph shows heat change versus time for 3R Tau combined with MAP4, with the vertical axis ranging from 0.00 to −0.20 microcalories per second and the horizontal axis ranging from 0 to 7000 seconds. The graph displays continuous titration data with small periodic peaks near the baseline, indicating negligible heat changes during the titrations of MAP4 into 3R Tau monomer solution. The negligible heat changes observed across all four titration combinations indicate that neither MAP2 nor MAP4 interacts directly with either 3R or 4R Tau monomers under the experimental conditions employed.

The polypeptide does not interact directly with Tau monomers as demonstrated by negligible heat changes in ITC titrations. The ITC titrations were carried out under conditions where twenty injections of Tau monomers, each of 2.5 μL spaced at intervals of 300 seconds, were added to 300 μL of cell volume containing 10 μM of MAP2 or MAP4 monomers. The concentrations of Tau monomers in the syringe were maintained at 70 μM such that at the last injection, the final concentration of Tau monomers remained at 10 μM, matching the stoichiometry used in the inhibition assays. Titrations of Tau monomers into buffer alone were used for subtracting heat of dilutions from the experimental measurements.

The polypeptide does not interact directly with Tau monomers as demonstrated by negligible heat changes in ITC titrations. The ITC titrations were carried out under conditions where twenty injections of Tau monomers, each of 2.5 μL spaced at intervals of 300 seconds, were added to 300 μL of cell volume containing 10 μM of MAP2 or MAP4 monomers. The concentrations of Tau monomers in the syringe were maintained at 70 μM such that at the last injection, the final concentration of Tau monomers remained at 10 μM, matching the stoichiometry used in the inhibition assays. Titrations of Tau monomers into buffer alone were used for subtracting heat of dilutions from the experimental measurements.

The negligible heat changes observed across all four titration combinations shown in FIG. 30 indicate absence of physiological interactions between Tau monomers and MAP proteins. The absence of detectable binding interactions between MAP proteins and Tau monomers in solution indicates that the inhibitory potential of MAP proteins may be attributed to their binding and capping potential to original or newly formed Tau seeds during aggregation reactions, rather than to sequestration or conformational modulation of the Tau monomer substrate. Since ITC is highly sensitive to binding or structural changes, the negligible heat changes rule out the possibility of MAPs-based sequestering or altering the conformational landscape of the Tau monomer pool.

The ITC titration data distinguishes between different mechanisms of inhibitory activity. The capping mechanism involves engagement of MAP proteins with fibril ends to block access for incoming Tau monomers, while a sequestration mechanism would involve binding of MAP proteins to Tau monomers in solution to deplete the pool of aggregation-competent substrate. The negligible heat changes rule out the sequestration mechanism, supporting the conclusion that MAP proteins exert inhibitory activity through direct engagement with fibril surfaces.

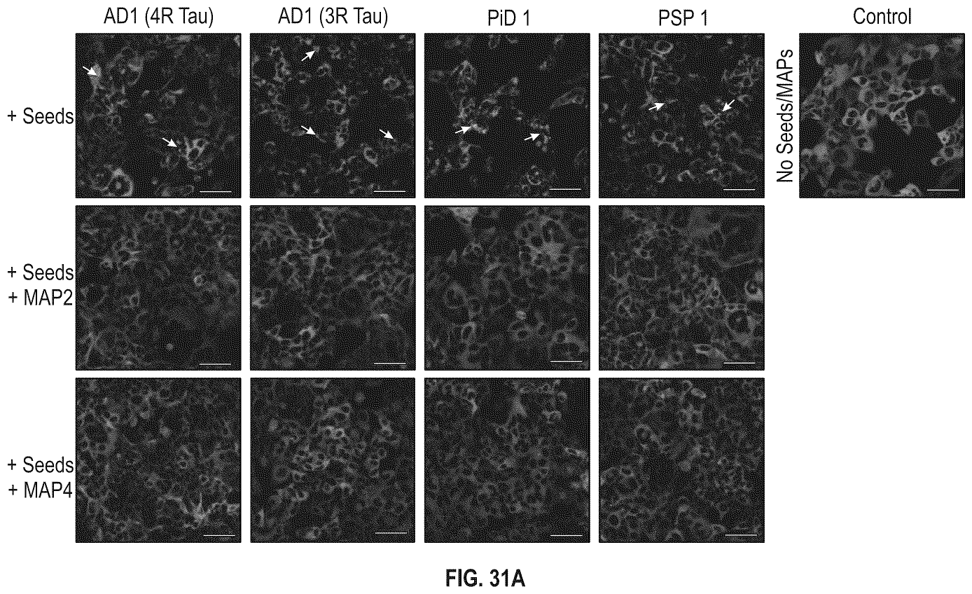

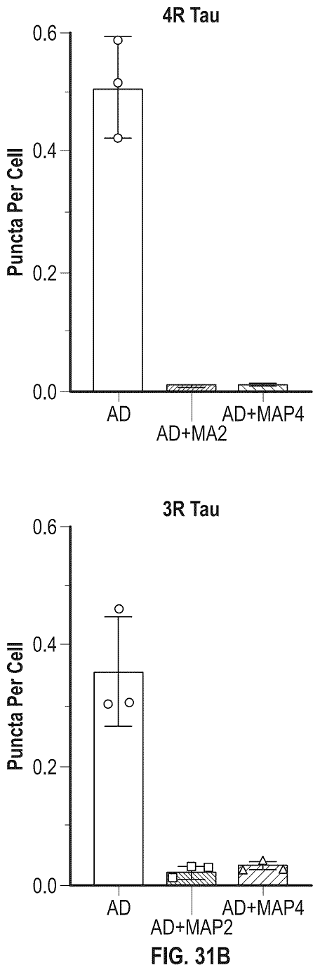

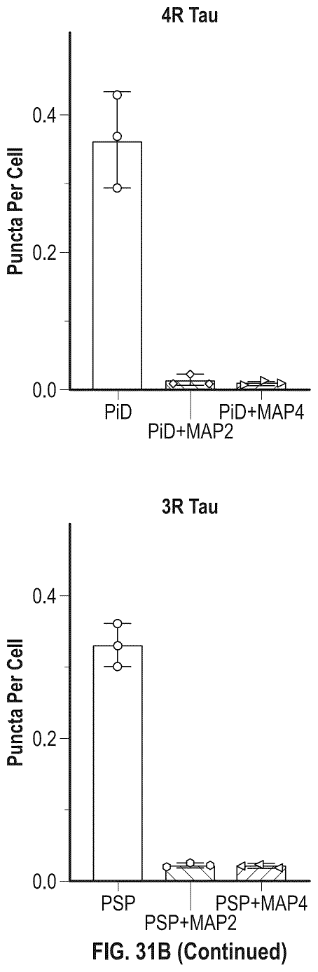

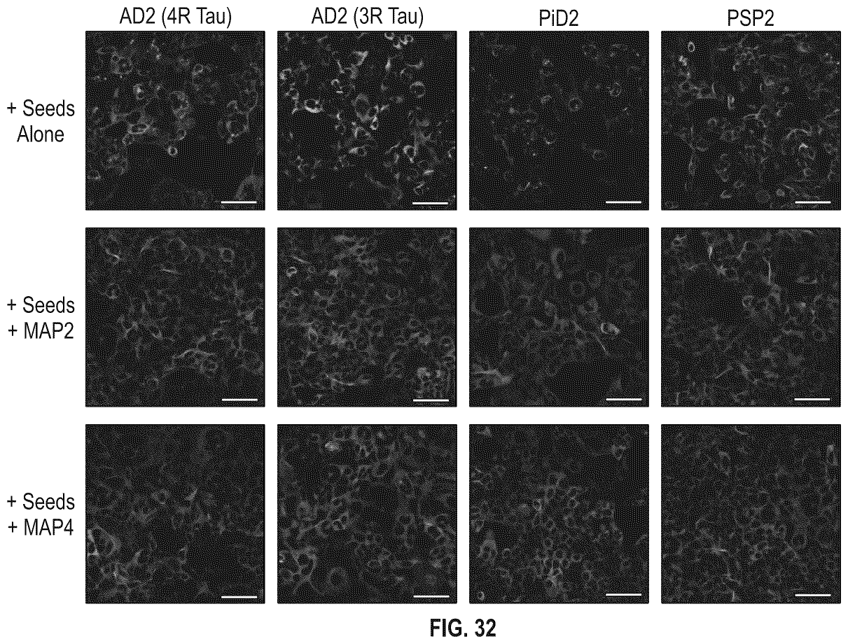

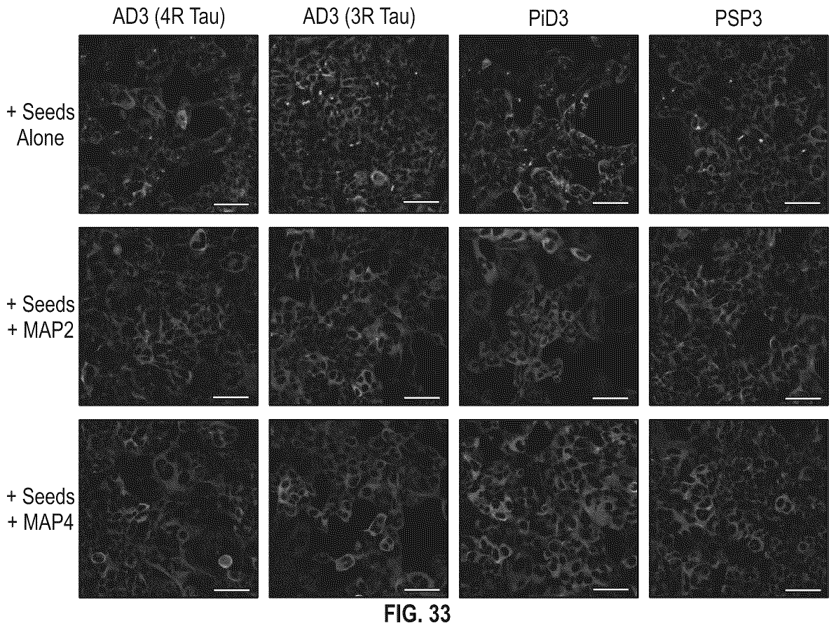

Referring to FIGS. 31, 32, and 33, representative fluorescent microscopy images of HEK293 cells transfected with seeds derived from RT-QuIC assays demonstrate the inhibitory effects of MAP2 and MAP4 on intracellular Tau aggregation. FIGS. 31, 32, and 33 correspond to three independent biological replicates (AD1/PiD1/PSP1, AD2/PiD2/PSP2, and AD3/PiD3/PSP3 respectively). Each figure is organized in a grid format with rows corresponding to different seed conditions (seeds alone, seeds with MAP2, seeds with MAP4) and columns corresponding to different disease samples including AD with 4R Tau, AD with 3R Tau, PiD, and PSP.

The HEK293 cells used in the assays are engineered to over-express P301S variant of Tau, which may grow and amplify on externally provided fibrils. P301S is a pathogenic Tau variant associated with frontotemporal dementia and parkinsonism linked to chromosome 17 (FTDP-17). The inhibitory activity of MAP2 and MAP4 demonstrated against this pathogenic variant indicates that the polypeptides may inhibit aggregation of both wild-type Tau isoforms and pathogenic Tau variants characterized by single amino acid substitutions or deletions associated with FTDP-17. Images were captured 24 hours post-transfection. Scale bars of 40 micrometers are provided in each image panel for reference.

Across all three figures, a consistent pattern is observed. In the seed-only conditions shown in the top rows, distinct puncta indicated by yellow arrows are visible within the cells, representing amyloid aggregation. The puncta appear as bright fluorescent foci distributed within the cellular cytoplasm, representing sites where seed-templated aggregation of the endogenously expressed Tau protein has occurred. In the MAP2-treated and MAP4-treated conditions shown in the middle and bottom rows, the cells show markedly reduced puncta formation, with more uniform fluorescence distribution resembling control cells without seed treatment. The control conditions show cells without seed treatment displaying uniform fluorescence without puncta formation.

Quantitative analysis presented in FIG. 31 displays bar graphs of puncta per cell for each disease condition. The polypeptide reduces puncta formation efficiency from approximately 30% with greater than 0.3 puncta per cell in seed-only conditions to approximately 5% efficiency in MAP-treated conditions. Error bars indicate standard deviation from three biological triplicates, demonstrating reproducibility of the quantitative assessment.

The reduction in puncta formation provides support for the ability of the polypeptide to decrease prion-like cell-to-cell propagation of Tau seeds. Tau filaments display prion-like transmission behavior, where pre-existing filaments catalyze recruitment of Tau monomers, driving propagation and contributing to spread of pathology. The cellular puncta assay models this prion-like seeding process by introducing disease-derived fibrillar seeds into cells expressing aggregation-prone Tau protein. By neutralizing the seeding competence of Tau fibrils prior to cellular uptake, the polypeptide may decrease prion-like propagation that would otherwise contribute to spread of Tau pathology. The consistent reduction across seeds from AD, PiD, and PSP brain samples indicates the polypeptide may interfere with prion-like propagation mechanisms across multiple tauopathies characterized by different fibril conformations.

The reduction in puncta formation demonstrates that the polypeptide may decrease prion-like cell-to-cell propagation of Tau seeds. By neutralizing the seeding competence of Tau fibrils prior to cellular uptake, the polypeptide may decrease prion-like propagation that would otherwise contribute to spread of Tau pathology. The consistent reduction across seeds from AD, PiD, and PSP brain samples indicates the polypeptide may interfere with prion-like propagation mechanisms across multiple tauopathies characterized by different fibril conformations.

In therapeutic applications, the polypeptide may be administered to a subject for treatment of a tauopathy. The tauopathies amenable to treatment include Alzheimer's disease, progressive supranuclear palsy, corticobasal degeneration, Pick's disease, argyrophilic grain disease, globular glial tauopathy, frontotemporal lobar degeneration with Tau, chronic traumatic encephalopathy, and frontotemporal dementia associated with MAPT mutation. The polypeptide may be formulated in a pharmaceutical composition including a pharmaceutically acceptable carrier, excipient, or diluent suitable for administration. The pharmaceutical composition may be formulated for administration by various routes including intravenous, subcutaneous, intrathecal, intracerebroventricular, intranasal, or convection enhanced delivery routes.

The polypeptide may be administered in an amount sufficient to reduce Tau-associated pathology in the subject. The reduction in Tau-associated pathology may be assessed through various measures including reduction in phosphorylated Tau levels, reduction in Tau seeding activity, decrease in Tau PET imaging signal, reduction in cerebrospinal fluid pTau181 or pTau217 levels, or improvement in cognitive or functional assessment relative to baseline. In some cases, the dosing may be adjusted according to pharmacokinetic parameters, anti-drug antibody titers, or biomarker response in the subject.

The pharmaceutical composition may be provided in a dosage form appropriate for weekly, biweekly, monthly, or quarterly dosing intervals. In some cases, the pharmaceutical composition may further include a nanoparticle, liposomal carrier, exosome-based system, polymeric depot, hydrogel formulation, or implantable device for sustained release. The pharmaceutical composition may be filled into a vial, prefilled syringe, or autoinjector device and labeled for use in treatment of a tauopathy.

The polypeptide may be modified to enhance pharmacological properties for therapeutic applications. In some cases, the polypeptide may be modified by PEG conjugation, glycoengineering, cyclization, stapling, or lipid attachment while retaining Tau inhibitory function. The polypeptide may further include a fusion partner, tag, half-life extending element, or brain delivery module such as an Fc region, albumin binding domain, blood-brain barrier shuttle peptide, transferrin receptor targeting domain, apolipoprotein mimetic sequence, FcRn enhancing mutation, or cell penetrating peptide. The polypeptide may be configured as a dimeric, multimeric, scaffold displayed, or Fc fused construct to increase binding affinity towards monomeric Tau or fibrillar Tau species.

In some cases, the polypeptide may be fused to an antibody or antibody fragment that binds a receptor mediating blood-brain barrier transport to facilitate central nervous system uptake. The fusion approach may enable delivery of the polypeptide capper to the brain where Tau pathology occurs, enhancing therapeutic efficacy by enabling direct engagement with disease-associated Tau fibrils in the central nervous system.

The polypeptide may also be delivered through gene therapy approaches. An isolated nucleic acid molecule encoding the polypeptide may be incorporated into an expression vector for delivery to a subject. The expression vector may be operably linked to a secretion signal sequence or a blood-brain barrier shuttle sequence enabling central nervous system delivery. A host cell containing the expression vector may be capable of expressing the encoded polypeptide. The polypeptide may be expressed within the central nervous system following administration of the expression vector, enabling endogenous production of the capper molecule at the site of Tau pathology.

In diagnostic applications, the polypeptide may be used for capture of pathological Tau filaments by immobilizing the polypeptide on a solid support to pull out aggregates from biological samples. The solid support may include a plate, bead, membrane, or microfluidic surface suitable for immobilization of the polypeptide. When a biological sample such as blood serum or cerebrospinal fluid is contacted with the immobilized polypeptide, aggregates present in the sample bind to the immobilized polypeptide due to the affinity between the polypeptide and fibrillar Tau ends. The binding interaction enables pullout and capture of aggregated Tau species from the biological sample, enriching aggregated species from complex biological matrices that may contain a mixture of monomeric and aggregated Tau forms. The immobilized polypeptide may selectively capture oligomeric or fibrillar Tau relative to monomeric Tau, enabling enrichment of aggregated Tau species from complex biological samples. The biological sample may include cerebrospinal fluid, plasma, serum, whole blood, saliva, urine, or a brain-derived extracellular vesicle preparation.

The polypeptide may be labeled with a detectable marker for diagnostic detection of Tau filaments. The detectable marker may include a fluorescent probe, enzymatic label, radioactive isotope, or other detection moiety that enables visualization or quantification of polypeptide binding to Tau aggregates. The labeled polypeptide may be used to detect the presence of aggregated Tau species in biological samples, providing diagnostic information about disease status or progression.

A diagnostic assay may include the polypeptide configured as a capture reagent in a sandwich format, further including a detection antibody or labeled binding partner recognizing Tau. The sandwich format enables quantification of Tau aggregates or Tau seeding activity in a biofluid sample obtained from a subject. The assay may quantify the level of bound Tau, where the level of bound Tau is indicative of the presence or extent of a tauopathy.

A method of stratifying a subject for risk or progression of a tauopathy may include incubating a biofluid sample from the subject with the polypeptide under conditions sufficient to bind aggregation-prone Tau species, measuring the extent of binding or capture, and correlating increased binding relative to a reference control with elevated disease burden or risk. The stratification approach may enable identification of subjects who may benefit from therapeutic intervention or who may be at elevated risk for disease progression.

A diagnostic kit may include the polypeptide immobilized on a plate, bead, membrane, or microfluidic surface, optionally together with reagents for detection of captured Tau including labeled antibodies, fluorescent probes, enzymatic substrates, or amplification reagents, and instructions for use in analyzing Tau species from cerebrospinal fluid or peripheral biofluid samples. The kit format may enable standardized diagnostic testing across multiple clinical sites or research laboratories.

The polypeptide may be used for assay development to isolate Tau material for research experiments. The capture capability of the polypeptide enables isolation of aggregated Tau species from biological samples for subsequent characterization through biochemical, biophysical, or structural methods. By immobilizing the polypeptide on a solid support and contacting the support with a biological sample containing aggregated Tau, the aggregates may be pulled out and enriched from the sample matrix. The isolated material may be used for experiments including structural analysis, seeding activity assessment, biochemical characterization, or evaluation of therapeutic candidates. The assay development application provides a tool for advancing understanding of Tau pathology and for developing new therapeutic approaches. In some cases, the capture approach may enable isolation of disease-specific Tau fibril conformations from patient samples for research purposes.

The homology-based approach provides advantages over antibody-based approaches in various embodiments for both therapeutic and diagnostic applications. The polypeptide constructs function analogously to antibodies in that they may capture target molecules, detect target molecules, and inhibit target molecules. However, the polypeptide constructs have the benefit that the protein itself is intrinsically disordered, which makes the constructs much easier to manipulate compared to antibodies. The intrinsically disordered nature of the polypeptide constructs enables site-directed mutagenesis to be performed in the core region, allowing rapid and cost-efficient tailoring of the constructs to enhance target range, selectivity, and interactive surface area. In contrast, antibodies are structured proteins where mutagenesis may disrupt the overall fold and binding properties. The polypeptide constructs are also smaller in molecular weight compared to antibodies, enabling easier production and potentially improved tissue penetration. The intrinsically disordered nature of the polypeptide constructs further enables conformational adaptability that allows engagement with different disease-associated Tau fold conformations, providing broad-spectrum activity against multiple tauopathies.

The homology-based approach provides advantages in various aspects for distinguishing between different Tau fibril conformations. While most antibodies detect small sequence-dependent epitopes, the polypeptide constructs engage with fibril surfaces through extensive contact spanning the entire cross-section of the fibril tip. The extensive contact surface enables detection of conformational features that may not be accessible to antibody-based approaches. Because the polypeptide constructs are intrinsically disordered, site-directed mutagenesis may be readily performed in the core region to selectively engage with one type of fibril conformation and not others, enabling development of disease-specific diagnostic reagents capable of distinguishing between different tauopathies based on differential binding properties.

The homology-based approach provides advantages in various embodiments for cost-effective development of diverse reagent sets. The development of monoclonal antibodies targeting specific Tau fibril conformers is slow and costly, with challenges relating to cross-reactivity and poorly understood conformational epitopes. The polypeptide constructs may be generated through recombinant expression in prokaryotic or eukaryotic host cells and purified using affinity chromatography, ion exchange chromatography, size exclusion chromatography, or combinations thereof to obtain therapeutic grade purity. The ease of production enables generation of diverse sets of polypeptide variants for screening and optimization.

The polypeptide constructs function analogously to antibodies in that the constructs may capture, detect, and inhibit target molecules, but the constructs achieve these functions through homology-based interactions rather than antigen-antibody binding. The homology-based mechanism enables engagement with Tau fibril surfaces through complementary structural interactions that span the fibril cross-section, providing extensive contact area that enhances binding strength and specificity. The conformational adaptability of the polypeptide constructs enables engagement with diverse disease-associated Tau fold conformations, supporting broad-spectrum activity against multiple tauopathies while maintaining the potential for disease-specific selectivity through rational mutagenesis.

The polypeptide constructs described herein may be produced through various methods including exogenous expression in bacterial systems and endogenous delivery via gene therapy approaches. The production flexibility enables selection of manufacturing approaches suited to particular therapeutic or diagnostic applications.

In exogenous production approaches, the polypeptide may be generated through solid-state synthesis or expression through a host organism. Solid-state peptide synthesis enables chemical construction of polypeptide sequences through sequential coupling of amino acid residues on a solid support, providing precise control over sequence composition and enabling incorporation of non-natural amino acids or chemical modifications. Expression through a host organism enables recombinant production of polypeptides through introduction of encoding nucleic acid sequences into cells capable of transcription and translation of the genetic information.

The polypeptide may be cloned in pET-28 vectors for recombinant expression. The pET-28 vector system provides a platform for high-level expression of recombinant proteins in bacterial hosts, with the vector containing regulatory elements that enable inducible expression of the cloned gene. In some aspects, gene-optimized DNA inserts coding for truncated constructs of human MAP2 and MAP4 may be synthesized and cloned into pET-28 expression vectors, with the genes inserted using Ncol and Xhol restriction sites with two stop codons preceding the Xhol site to eliminate all tags. The pET-28 vector includes a T7 promoter that enables transcription of the cloned gene when the host cell expresses T7 RNA polymerase, providing tight control over expression timing and level. Since MAP2 and MAP4 are intrinsically disordered proteins, they may be purified using heat denaturation methods similar to those used for Tau protein, where the proteins remain in the supernatant upon heating at 80-100° C., followed by precipitation with ammonium sulfate.

The polypeptide may be cloned in any expression vectors suitable for recombinant protein production in bacteria or other hosts.

The polypeptide may be expressed in various host systems suitable for recombinant protein production, including but not limited to bacterial, insect, yeast, and mammalian expression systems. In some aspects, bacterial expression systems may be used following transformation with expression vectors. Suitable bacterial hosts include, but are not limited to, Escherichia coli strains such as BL21 (DE3), BL21 (DE3) pLysS, Rosetta, Rosetta 2, Origami, SHuffle, C41 (DE3), C43 (DE3), or other protein expression strains, as well as other bacterial species capable of recombinant protein production.

The protein may be purified using affinity chromatography, ion exchange, size exclusion, heat stability, or any other methods used in isolation or combined.

In endogenous production approaches, the polypeptide may be generated in the brain through introduction of genetic information via vectors, RNA, or CRISPR-based methods. The endogenous production approach enables expression of the polypeptide capper at the site of Tau pathology in the central nervous system, potentially enhancing therapeutic efficacy by enabling sustained local production of the inhibitory molecule.

Viral vectors may be used to deliver nucleic acid sequences encoding the polypeptide to cells in the central nervous system. Adeno-associated virus (AAV) vectors provide a platform for gene delivery to the brain with favorable safety profiles and the ability to achieve long-term expression in post-mitotic neurons. Lentiviral vectors may also be employed for gene delivery, with the ability to integrate into the host genome and provide stable long-term expression. The vector may be engineered to include tissue-specific promoters that restrict expression to particular cell types in the central nervous system.

RNA-based delivery approaches may be used to introduce genetic information encoding the polypeptide into cells. Messenger RNA (mRNA) encoding the polypeptide may be delivered to cells, enabling transient expression of the capper molecule without permanent modification of the cellular genome. The mRNA may be modified with chemical modifications that enhance stability and reduce immunogenicity. Lipid nanoparticle formulations may be used to facilitate cellular uptake of the mRNA and protect the nucleic acid from degradation.

CRISPR-based approaches may be used to introduce genetic information encoding the polypeptide into the cellular genome. CRISPR-Cas systems enable targeted insertion of nucleic acid sequences at specific genomic locations, providing precise control over the site of integration. The CRISPR approach may enable stable long-term expression of the polypeptide through integration of the encoding sequence into the host genome under control of endogenous or exogenous regulatory elements.

The polypeptide may be enhanced by protein modifications including phosphorylation, acetylation, or PEGylation. Post-translational modifications may alter the properties of the polypeptide including binding affinity, stability, solubility, immunogenicity, and pharmacokinetic behavior. The modifications may be introduced during or after polypeptide production depending on the specific modification and production method employed.

Phosphorylation involves addition of phosphate groups to serine, threonine, or tyrosine residues in the polypeptide sequence. Phosphorylation may be introduced through enzymatic treatment with kinases that catalyze transfer of phosphate groups from ATP to specific residue positions. Phosphorylation may alter the charge properties and conformational behavior of the polypeptide, potentially affecting binding affinity for Tau fibril surfaces. In some cases, phosphomimetic mutations such as substitution of serine or threonine with aspartate or glutamate may be introduced to mimic the effects of phosphorylation without requiring enzymatic modification.

Acetylation involves addition of acetyl groups to lysine residues or the amino terminus of the polypeptide. Acetylation may be introduced through chemical treatment with acetylating reagents or through enzymatic treatment with acetyltransferases. Acetylation neutralizes the positive charge of lysine residues, potentially affecting electrostatic interactions between the polypeptide and Tau fibril surfaces. Acetylation may also affect protein stability and resistance to proteolytic degradation.

PEGylation involves covalent attachment of polyethylene glycol (PEG) polymers to the polypeptide. PEGylation may be achieved through reaction of activated PEG reagents with amino groups, thiol groups, or other reactive functional groups on the polypeptide surface. PEGylation increases the hydrodynamic radius of the polypeptide, reducing renal clearance and extending serum half-life. PEGylation may also reduce immunogenicity by shielding the polypeptide surface from recognition by the immune system. The size and architecture of the PEG polymer may be selected to achieve desired pharmacokinetic properties while maintaining Tau inhibitory function.

The production flexibility provided by multiple manufacturing approaches enables selection of methods suited to particular applications. Bacterial expression provides a cost-effective approach for producing large quantities of polypeptide for research applications and initial therapeutic development. Mammalian cell expression may be employed when post-translational modifications characteristic of mammalian systems are desired. Solid-state synthesis enables incorporation of non-natural amino acids and site-specific chemical modifications that may not be achievable through recombinant expression. Gene therapy approaches enable endogenous production at the site of pathology, potentially providing sustained therapeutic benefit through continuous local expression of the capper molecule.

The production methods may be combined with modification approaches to generate polypeptide constructs with tailored properties for specific applications. In some cases, recombinantly expressed polypeptides may be subjected to enzymatic or chemical modification to introduce phosphorylation, acetylation, or PEGylation. In other cases, modified amino acids may be incorporated during solid-state synthesis to introduce modifications at specific positions. The combination of production and modification approaches provides a flexible platform for developing polypeptide constructs optimized for therapeutic efficacy, pharmacokinetic behavior, and manufacturing feasibility.

The polypeptide constructs described herein encompass variant sequences that share between 20% and 99% sequence similarity with the structured core regions of pathological Tau filaments. The range of sequence similarity enables development of polypeptide constructs with varying degrees of structural relatedness to native Tau protein while maintaining the ability to engage with fibril surfaces and inhibit aggregation. The lower end of the sequence similarity range, such as 20%, allows for substantial changes in the sequence without departing from the homology-based capping approach. The upper end of the sequence similarity range, such as up to 99%, encompasses Tau variants with one or two mutations that may also serve as caps.

A Tau variant with one or two mutations may serve as a cap with up to 99% sequence similarity to native Tau protein. The high sequence similarity between such Tau variants and native Tau enables engagement with fibril ends through complementary interactions that closely mimic native Tau-Tau interactions. However, the one or two mutations at specific positions may prevent the Tau variant from supporting elongation of the fibril, thereby enabling the variant to function as a terminal cap that blocks recruitment of additional Tau monomers. The identification of positions where single or double mutations confer capping activity without eliminating fibril engagement may be informed by saturation mutagenesis mapping and molecular dynamics simulations as described herein.

A Tau variant with one or two mutations may serve as a cap with up to 99% sequence similarity to native Tau protein. The high sequence similarity between such Tau variants and native Tau enables engagement with fibril ends through complementary interactions that closely mimic native Tau-Tau interactions. However, the one or two mutations at specific positions may prevent the Tau variant from supporting elongation of the fibril, thereby enabling the variant to function as a terminal cap that blocks recruitment of additional Tau monomers. The identification of positions where single or double mutations confer capping activity without eliminating fibril engagement may be informed by saturation mutagenesis mapping and molecular dynamics simulations as described herein.

Referring to FIGS. 12, 13, and 14, molecular dynamics simulations of MAP2 and MAP4 modeled as multi-layered assemblies in AD, PiD, and PSP fibril folds respectively are presented. Each figure includes panel a showing a graph of Ca-RMSD in nanometers plotted against time in nanoseconds over a 1000 ns simulation period, with separate traces for Tau, MAP2, and MAP4. Each figure also includes panel b presenting structural snapshots extracted from simulation trajectories at time points of 0, 250, 500, 750, and 1000 nanoseconds for Tau, MAP2, and MAP4 systems.

Across all three disease-associated folds, a consistent pattern emerges from the simulations. Tau maintains relatively stable RMSD values of approximately 0.3 to 0.5 nm throughout the simulation period and retains its fibril fold architecture in the structural snapshots. In contrast, MAP2 shows elevated RMSD values of approximately 1.0 to 1.5 nm, and MAP4 shows even higher RMSD values of approximately 1.5 to 2.5 nm. Both MAP proteins exhibit progressive structural destabilization in the snapshots, with marked deviation from the starting disease-associated fold configurations.

The inability of MAP proteins to sustain disease-associated fold architectures in multi-layered assemblies indicates that they may not template further growth of MAP or Tau monomers once engaged with a fibril end. This contrasts with the stable capping behavior demonstrated when MAP proteins are docked as single layers onto existing Tau fibril surfaces, as shown in FIGS. 2A-2C and FIG. 11. The structural destabilization provides a mechanistic basis for the capping behavior observed in experimental assays, where MAP proteins inhibit Tau aggregation without self-aggregating or forming heteromolecular MAP-Tau fibril assemblies.

The consistent pattern of MAP destabilization across AD, PiD, and PSP fold contexts indicates that the capping mechanism may operate through a common structural basis regardless of the specific disease-associated fold conformation, supporting development of polypeptide constructs with activity against multiple tauopathies.

The sequence identity range may extend below 49% down to approximately 20% based on the saturation mutagenesis analysis shown in FIG. 7. The saturation mutagenesis map reveals that many positions in the MAP2 sequence tolerate substitution while maintaining binding affinity, with stabilizing mutations distributed across multiple positions. The inventors observed that residues forming the attractive intermolecular contact network between Tau layers were mostly conserved or substituted with similar residues in MAP proteins, while nonconserved regions may not stack in the original arrangement and may change fibril morphology, preventing recruitment of further Tau on altered surfaces. This indicates that only a subset of residue positions-those contributing to the attractive contact network at the fibril interface—may need to be conserved for capping activity, while remaining positions may tolerate substantial substitution. Accordingly, polypeptide constructs with sequence identity as low as approximately 20% may retain capping activity provided that residues at positions contributing to the intermolecular contact network with the fibril surface are conserved or substituted with residues that maintain favorable interactions.

The sequence identity range may extend up to 99% because a Tau variant with as few as one or two mutations at positions that disrupt inter-layer stacking but preserve fibril-end binding may function as a terminal cap. The molecular dynamics simulations demonstrate that MAP proteins can bind stably to fibril ends as a single layer but cannot sustain multi-layered assemblies, indicating that specific residue differences at inter-layer contact positions may be sufficient to confer capping behavior. A polypeptide sharing up to 99% sequence identity with SEQ ID NO:1 or SEQ ID NO:2—differing by as few as one or two residues—may retain the ability to bind to fibril ends while the mutation or mutations prevent the polypeptide from supporting further stacking of additional layers.

The sequence identity value for a given polypeptide construct may depend on the specific comparison being made. For example, the sequence identity may vary depending on whether the comparison involves 3R with 3R, 4R with 4R, or 3R with 4R repeat regions, as the number of shared residues varies across these comparisons. The experimentally demonstrated inhibitory activity of MAP2 and MAP4 across multiple tauopathies, combined with the saturation mutagenesis data indicating position-specific tolerance for substitution, supports the 20% to 99% sequence identity range for polypeptide constructs that retain functional capping activity.

The polypeptide may be altered to selectively cap one type of filament and not others for targeted therapies. Since Tau fibrils involved in different tauopathies present distinct structural conformations with varying cross-sectional packing interactions due to different meanderings of the Tau backbone, polypeptide variants may be designed to engage preferentially with specific fibril conformations. The selective capping approach enables development of therapeutic agents with activity against particular tauopathies while sparing fibril conformations associated with other conditions. In some cases, selective cappers may be designed by incorporating mutations that enhance binding to a target fibril conformation while reducing binding to non-target conformations.

The selective capping approach may be achieved through rational mutagenesis guided by structural analysis of different disease-associated fibril conformations. The cryo-EM structures of Tau fibrils from Alzheimer's disease, Pick's disease, progressive supranuclear palsy, and other tauopathies reveal distinct structural topologies with different residue contacts and packing arrangements. Polypeptide variants may be designed to complement the specific contact patterns of a target fibril conformation while introducing steric clashes or unfavorable interactions with non-target conformations. The saturation mutagenesis mapping approach enables systematic identification of mutations that differentially affect binding to different fibril types.

The extensive binding surface provided by homology-based polypeptide cappers spans the entire cross-section of the fibril tip. The binding surface extends across the complete width of the fibril end, providing contact area that encompasses all residue positions in the structured core region of the Tau filament. The extensive contact surface contrasts with the limited contact surfaces available to small molecule inhibitors, which typically engage with only a subset of residue positions on the fibril surface. The extensive binding surface also contrasts with antibody-based approaches, where the antibody paratope engages with a localized epitope rather than spanning the entire fibril cross-section.

The extensive binding surface provides advantages in various embodiments for achieving high-affinity engagement with fibril ends. The large contact area enables formation of numerous intermolecular contacts between the polypeptide capper and the fibril surface, with each contact contributing to the overall binding energy. The cumulative effect of multiple contacts across the fibril cross-section may result in binding affinities comparable to or exceeding native Tau-Tau interactions within the fibril structure. The high binding affinity enables effective competition with Tau monomers for fibril end binding sites, supporting the capping mechanism whereby the polypeptide occupies the fibril end and blocks access for incoming monomers.

The extensive binding surface provides advantages in various aspects for conformational selectivity. The binding interaction involves the entire cross-sectional topology of the fibril end, enabling discrimination between fibril conformations that differ in their overall structural architecture rather than only at localized epitope positions. The conformational selectivity enabled by the extensive binding surface may allow polypeptide cappers to distinguish between fibril types that share similar sequence epitopes but differ in their three-dimensional fold topology. In some cases, the conformational selectivity may enable development of diagnostic reagents capable of distinguishing between different tauopathies based on differential binding to disease-specific fibril conformations.

The tailored specificity of variant polypeptides provides advantages in various embodiments for therapeutic applications targeting specific tauopathies. A polypeptide variant designed to selectively cap Alzheimer's disease-associated fibrils may be administered to subjects with Alzheimer's disease without affecting fibril conformations that may serve physiological functions or that may be associated with other conditions. The selective approach may reduce potential off-target effects by limiting activity to the disease-relevant fibril population. In some cases, the selective capping approach may enable combination therapies where multiple polypeptide variants with different selectivity profiles are administered to address heterogeneous fibril populations within a single patient.

The tailored specificity provides advantages in various aspects for diagnostic applications requiring discrimination between different tauopathies. A panel of polypeptide variants with different selectivity profiles may be used to characterize the fibril composition of biological samples, enabling classification of samples based on the pattern of binding to different polypeptide variants. The diagnostic panel approach may enable distinction between tauopathies that present similar clinical features but differ in their underlying fibril pathology. In some cases, the diagnostic information may guide selection of therapeutic approaches tailored to the specific fibril type present in a given patient.

The variant polypeptides may be generated through site-directed mutagenesis of MAP2, MAP4, or Tau sequences. Site-directed mutagenesis enables precise introduction of amino acid substitutions at specific positions in the polypeptide sequence, providing control over the location and nature of sequence modifications. The mutagenesis approach may be guided by computational predictions from saturation mutagenesis mapping, structural analysis of fibril conformations, or empirical screening of variant libraries. In some cases, combinations of multiple mutations may be introduced to achieve additive or synergistic effects on binding affinity or selectivity.

The variant polypeptides may retain the intrinsically disordered character of the parent MAP2, MAP4, or Tau sequences while incorporating mutations that modulate binding properties. The intrinsically disordered character enables conformational flexibility that allows the polypeptide to adapt to the structural topology of the target fibril surface upon engagement. The mutations may affect the conformational preferences of the polypeptide in the bound state, altering the complementarity between the polypeptide and specific fibril conformations. In some cases, mutations may be selected to enhance complementarity with a target fibril conformation while reducing complementarity with non-target conformations.

The variant polypeptides encompass truncated versions of MAP2, MAP4, or Tau protein that include only the core region responsible for fibril interaction. The truncated constructs may use specific regions corresponding to the microtubule binding repeat domains that contribute to the high sequence similarity with 3R or 4R Tau. The truncation approach removes regions that do not contribute to fibril engagement, potentially improving pharmacological properties by reducing molecular weight and eliminating sequences that may contribute to immunogenicity or off-target interactions. The truncated variants retain the structured core that engages with disease-associated fibril ends while excluding flanking regions.

The variant polypeptides may be designed as consensus sequences, chimeric constructs, humanized forms, or de-immunized variants. Consensus sequences may be derived from alignment of multiple MAP or Tau sequences to identify conserved residue positions that contribute to fibril engagement. Chimeric constructs may combine regions from different MAP or Tau proteins to achieve desired binding properties. Humanized forms may incorporate modifications that reduce predicted immunogenicity in human subjects. De-immunized variants may be designed to reduce predicted human T cell epitopes while maintaining Tau inhibitory activity.

The selective capping approach provides advantages in various embodiments for understanding the molecular basis of fibril-specific pathology. Polypeptide variants with defined selectivity profiles may be used as research tools to probe the contribution of specific fibril conformations to disease pathology. The selective cappers may enable experiments that distinguish between effects mediated by different fibril populations within heterogeneous samples. In some cases, the selective capping approach may reveal that particular fibril conformations contribute disproportionately to specific aspects of disease pathology, informing development of targeted therapeutic strategies.

A number of implementations have been described. Nevertheless, it will be understood that various modifications may be made without departing from the spirit and scope of the disclosure. Accordingly, other implementations are within the scope of the following claims.

While the disclosure herein discusses details of exemplary embodiments of homology-based polypeptide cappers for inhibiting Tau aggregation and associated processes, additional modifications may be contemplated and are considered to be a part of the present disclosure. For example, one or more of the components of the RT-QuIC assay workflow described herein may be automated using, for example, a robot system such that at least a portion of the experimental workflow may be fully automated. Alternative methods of providing the shaking, quaking, and/or fibril breakage may be used in place of those discussed above. The computational workflows for analyzing polypeptide binding to Tau fibrils, including homology modeling, molecular dynamics simulations, and saturation mutagenesis mapping, may be implemented using alternative software platforms or computational approaches.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of various embodiments of any apparatus embodiment, a method or process embodiment, or even merely a variation of any element of these. Particularly, it should be understood that the words for each element may be expressed by equivalent apparatus terms or method terms, even if only the function or result is the same. Such equivalent, broader, or even more generic terms should be considered to be encompassed in the description of each element or action. Such terms can be substituted where desired to make explicit the implicitly broad coverage to which this disclosure is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “polypeptide capper” should be understood to encompass disclosure of the act of “capping,” whether explicitly discussed or not, and, conversely, were there only disclosure of the act of “capping,” such a disclosure should be understood to encompass disclosure of a “polypeptide capper.” Such changes and alternative terms are to be understood to be explicitly included in the description.

1. A polypeptide capable of inhibiting Tau fibril elongation, the polypeptide comprising:
an amino acid sequence exhibiting between 20% and 99% sequence identity to SEQ ID NO: 1 corresponding to MAP2 or SEQ ID NO:2 corresponding to MAP4;
wherein the polypeptide is capable of binding to an end of a Tau fibril and forming a terminal layer cap that reduces Tau aggregation or Tau seeding activity, and wherein the polypeptide does not self-propagate or template further growth of Tau monomers or MAP monomers onto the fibril.

2. The polypeptide of claim 1, wherein the amino acid sequence comprises one or more amino acid substitutions relative to SEQ ID NO:1 or SEQ ID NO:2, and wherein the polypeptide retains functional activity in reducing Tau aggregation or Tau seeding activity.

3. The polypeptide of claim 1, wherein the polypeptide comprises a contiguous fragment of at least 2 amino acids derived from SEQ ID NO:1 or SEQ ID NO:2 that retains measurable Tau inhibitory activity.

4. The polypeptide of claim 1, wherein the polypeptide decreases prion-like cell-to-cell propagation of Tau seeds.

5. The polypeptide of claim 1, wherein the polypeptide is capable of adopting a disease-associated Tau fold conformation upon binding to the end of the Tau fibril.

6. The polypeptide of claim 1, wherein the polypeptide inhibits aggregation of both three-repeat (3R) Tau isoforms and four-repeat (4R) Tau isoforms, including wild-type Tau and pathogenic Tau variants characterized by single amino acid substitutions or deletions associated with frontotemporal dementia and parkinsonism linked to chromosome 17 (FTDP-17).

7. The polypeptide of claim 1, further comprising a fusion partner selected from the group consisting of an Fc region, an albumin binding domain, a blood-brain barrier shuttle peptide, a transferrin receptor targeting domain, and a cell penetrating peptide.

8. The polypeptide of claim 1, formulated as a pharmaceutical composition with a pharmaceutically acceptable carrier.

9. The pharmaceutical composition of claim 8, wherein the pharmaceutical composition is formulated for at least one of intravenous, subcutaneous, intrathecal, intracerebroventricular, intranasal, or convection-enhanced delivery.

10. A method for treating a tauopathy in a subject, the method comprising:
administering to the subject a therapeutically effective amount of a polypeptide comprising an amino acid sequence exhibiting between 20% and 99% sequence identity to SEQ ID NO: 1 corresponding to MAP2 or SEQ ID NO:2 corresponding to MAP4, wherein the polypeptide binds to ends of Tau fibrils in the subject and reduces Tau-associated pathology.

11. The method of claim 10, wherein the tauopathy comprises at least one of Alzheimer's disease, progressive supranuclear palsy, corticobasal degeneration, Pick's disease, argyrophilic grain disease, globular glial tauopathy, frontotemporal lobar degeneration with Tau, chronic traumatic encephalopathy, or frontotemporal dementia associated with MAPT mutation.

12. The method of claim 10, wherein the polypeptide is modified by at least one of polyethylene glycol (PEG) conjugation, glycoengineering, cyclization, stapling, or lipid attachment, and wherein the polypeptide retains Tau inhibitory function following the modification.

13. The method of claim 10, further comprising monitoring therapeutic response by measuring at least one of a reduction in phosphorylated Tau levels, a reduction in Tau seeding activity, a reduction in Tau positron emission tomography (PET) imaging signal, or an improvement in a cognitive or functional assessment relative to baseline.

14. The method of claim 10, wherein the polypeptide is fused to an antibody or antibody fragment that binds a receptor mediating blood-brain barrier transport, facilitating central nervous system uptake.

15. The method of claim 10, wherein the polypeptide is engineered as a consensus sequence, chimeric construct, humanized form, or de-immunized variant that reduces predicted human T cell epitopes while retaining activity against Tau aggregation.

16. A method for detecting aggregated Tau species in a biological sample, the method comprising:
(a) contacting the biological sample with a polypeptide immobilized on a solid support, wherein the polypeptide comprises an amino acid sequence exhibiting between 20% and 99% sequence identity to SEQ ID NO: 1 corresponding to MAP2 or SEQ ID NO:2 corresponding to MAP4;
(b) binding aggregated Tau species present in the biological sample to the immobilized polypeptide; and
(c) detecting bound Tau species;
wherein a level of bound Tau species indicates the presence or extent of a tauopathy in a subject from which the biological sample was obtained.

17. The method of claim 16, wherein the biological sample comprises at least one of cerebrospinal fluid, plasma, serum, whole blood, saliva, urine, or a brain-derived extracellular vesicle preparation.

18. The method of claim 16, wherein the immobilized polypeptide selectively captures oligomeric or fibrillar Tau relative to monomeric Tau.

19. The method of claim 16, further comprising a detection antibody or labeled binding partner recognizing Tau, wherein the immobilized polypeptide and the detection antibody or labeled binding partner are configured in a sandwich format that quantifies Tau aggregates or Tau seeding activity in the biological sample.

20. The method of claim 16, further comprising contacting the biological sample with a plurality of polypeptide variants, each variant comprising an amino acid sequence exhibiting between 20% and 99% sequence identity to SEQ ID NO:1 or SEQ ID NO:2, wherein the plurality of polypeptide variants exhibit differential binding to Tau fibril conformations associated with different tauopathies, and wherein a pattern of binding across the plurality of polypeptide variants is used to distinguish between tauopathies in the subject from which the biological sample was obtained.
